(19) United States
(12) Patent Application Publication
(10) Pub. No.: US 2026/0345617 A1
(43) Pub. Date: Nov. 5, 2026

(54) INHIBITORS OF EXONUCLEASE 1

(71) Applicant: CITY OF HOPE, Duarte, CA (US)

(72) Inventors: Binghui SHEN, La Verne, CA (US); Li ZHENG, Arcadia, CA (US); Mian ZHOU, Azusa, CA (US); Holly YIN, Arcadia, CA (US)

(73) Assignee: CITY OF HOPE, Duarte, CA (US)

(21) Appl. No.: 19/650,676

(22) Filed: Apr. 17, 2026

Related U.S. Application Data
(60) Provisional application No. 63/790,939, filed on Apr. 18, 2025.

(51) Int. Cl. A61K 31/519; A61K 31/451; A61K 31/495; A61K 31/50; A61K 31/505; A61K 31/53; C07D 211/14; C07D 233/96; C07D 295/155; C07D 487/04

(52) U.S. Cl. CPC A61K 31/519; A61K 31/451; A61K 31/495; A61K 31/50; A61K 31/505; A61K 31/53; C07D 211/14; C07D 233/96; C07D 295/155; C07D 487/04

(57) ABSTRACT

Disclosed herein are effective small molecule inhibitors that are selective for exonuclease 1 (EXO1). EXO1 is a key enzyme in the critical end resection step for the DNA BSB repair. EXO1 knockout results in synthetic lethality with BRCA1 deficiency, which frequently occurs in human breast, ovarian, and prostate cancers. EXO1 deletion also causes cancer cells more sensitive to DNA damaging agents. Also disclosed are pharmaceutical compositions, kits and methods of treatment for EXO1 related diseases.

### CROSS-REFERENCE TO RELATED APPLICATIONS

This application is based on and claims priority to U.S. Provisional Application No. 63/790,939, filed on Apr. 18, 2025, which is incorporated herein by reference in its entirety.

### REFERENCE TO A SEQUENCE LISTING SUBMITTED AS XML VIA EFS-WEB

The instant application contains a Sequence Listing XML which has been submitted electronically in XML format and is hereby incorporated by reference in its entirety. Said XML copy, created on Apr. 15, 2026, is named CYH-007US1_2026-04-15_Sequence_Listing_116437-1546656.xml and is 20,104 bytes in size.

### BACKGROUND

DNA double strand breaks (DSBs) are the most lethal type of DNA lesions. However, robust DSB repair leads cancer cells to escape from DSB induced lethal condition. Therefore, inhibition of DSB repair pathways is an attractive strategy to kill cancer cells and to sensitize cancer cells to radiotherapies and chemotherapies. Exonuclease 1 (EXO1) is a key enzyme in the critical end resection step for the DNA BSB repair. EXO1 knockout results in synthetic lethality with BRCA1 deficiency, which frequently occurs in human breast, ovarian, and prostate cancers. EXO1 deletion also causes cancer cells more sensitive to DNA damaging agents. There remains an unmet need for the development of small molecule inhibitors of EXO1 to for the treatment of cancer.

### SUMMARY

Disclosed herein are small molecule compounds, with specificity and efficacy as inhibitors of human exonuclease 1 (EXO1). This disclosed technology can be applied to cancer treatment.

Accordingly, the present disclosure includes human exonuclease 1 inhibitor compounds of Formula (I):

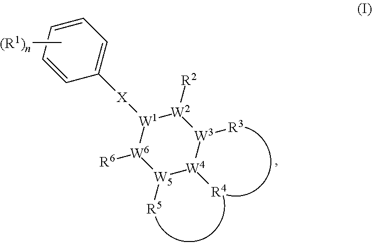

or a pharmaceutically acceptable salt thereof;
wherein X is a bond, —NH—, or —NH— alkyl-;
R¹ is absent, H, halo, or L;
R² absent, H, halo, or L;
R³ is H, halo, —OH, alkyl, bound to R⁴, or L;
R⁴ is H, halo, —OH, alkyl-L, bound to R³, bound to R⁵, or L;
R⁵ is absent, H, ═O, —C(O)O-alkyl, bound to R⁴, or L;
R⁶ is absent, H, or L;
W² is —CH—, —N═, —NH—;
W³ is —CH—, —C═;
W⁴ is —N—, —CH—, —C═;
W⁵ is —CH—, —C—, —C═;
W⁶ is —CH—, —C═, —N═;
L is ═O, —C(O)OH, alkyl, aryl, benzyl, —O-alkyl, —NHC(O)Y, —NHS(O)₂Y, —SCH₂C(O)O—Y;
Y is substituted or unsubstituted aryl, substituted or unsubstituted heteroaryl, substituted or unsubstituted heterocycle,
when R³ is bound to R⁴ or R⁴ is bound to R⁵ a five membered unsubstituted or L-20 substituted heterocycle is formed; and
n is 0-5.

In some cases, described herein are pharmaceutical compositions comprising the disclosed human exonuclease 1 inhibitor compounds and a pharmaceutically acceptable excipient.

Further disclosed are methods of treating a subject by administering to the subject the human exonuclease 1 inhibitors described herein. In some cases, the method is for treating cancer. In some cases, the method is for inhibiting cancer cell proliferation, cancer cell viability or a combination thereof.

In another further aspect, the present disclosure may be directed to a kit, comprising the human exonuclease 1 inhibitor compound and/or a pharmaceutical composition described herein.

### BRIEF DESCRIPTION OF THE DRAWINGS

FIG. 1 shows a box and whisker plot comparison of exonuclease 1 (EXO1) mRNA expression profiles in cancer and corresponding normal human tissues from the The Cancer Genome Atlas (TCGA) database.

FIG. 2 shows a volcano map depicting the correlation coefficients for the linear relationship between EXO1, Fanconi anemia, and cell cycle pathway gene expression.

FIG. 3 shows bar graph representing the Kyoto Encyclopedia of Genomics (KEGG) pathway enrichment analysis highlighting gene pathways that are co-dependent on EXO1.

FIG. 4 shows bar graph representing the KEGG pathway enrichment analysis highlighting gene pathways that are negatively correlated with EXO1 expression.

FIG. 5 shows a schematic of an assay to determine the IC₅₀ of inhibitor scaffolds against EXO1 protein via fluorescence resonance energy transfer (FRET)-Based high-throughput screen (HTS) assay.

FIG. 6 shows a graph plotting relative fluorescence unit (RFU) versus time for reactions with 100 nM DNA substrates and varying concentrations of EXO1.

FIG. 7 shows a graph plotting relative fluorescence unit (RFU) versus time for reactions with 100 pM EXO1 and varying concentrations of DNA substrates.

FIG. 8 shows a graph plotting relative fluorescence unit (RFU) versus time in reactions with varying concentrations of dimethyl sulfoxide (DMSO).

FIG. 9 shows a graph plotting the normalized relative fluorescence unit (RFU) versus time in reactions with or without the addition of varying concentrations of DMSO.

FIG. 10 shows a graph plotting relative fluorescence unit (RFU) versus time to demonstrate the stability of EXO1 activity.

FIG. 11 shows a graph plotting percent normalized relative fluorescence unit (RFU) % vs time comparing 0 h to 16 h demonstrating the stability of the EXO1 protein.

FIG. 12 shows a dose-response curve plotting relative fluorescence unit (RFU) versus concentration (M) and the IC₅₀ of inhibitor compound C200 against the EXO1 protein.

FIG. 13 shows a dose-response curve plotting relative fluorescence unit (RFU) versus concentration (M) and the IC₅₀ of inhibitor compound F684 against the EXO1 protein.

FIG. 14 shows a dose-response curve plotting relative fluorescence unit (RFU) versus concentration (M) and the IC₅₀ of inhibitor compound F685 against the EXO1 protein.

FIG. 15 shows a dose-response curve plotting relative fluorescence unit (RFU) versus concentration (M) and the IC₅₀ of inhibitor compound D351 against the EXO1 protein.

FIG. 16 shows a dose-response curve plotting relative fluorescence unit (RFU) versus concentration (M) and the IC₅₀ of inhibitor compound D431 against the EXO1 protein.

FIG. 17 shows a dose-response curve plotting relative fluorescence unit (RFU) versus concentration (M) and the IC₅₀ of inhibitor compound G889 against the EXO1 protein.

FIG. 18 shows a dose-response curve plotting relative fluorescence unit (RFU) versus concentration (M) and the IC₅₀ of inhibitor compound F571 against the EXO1 protein.

FIG. 19 shows a Richardson Diagram comparing the three-dimensional structure of the N-terminal domains of the EXO1 protein vs the FEN1 protein.

FIG. 20 shows a dose-response curve plotting relative fluorescence unit (RFU) versus concentration (M) and the IC₅₀ of inhibitor compound C200 against the FEN1 protein.

FIG. 21 shows a dose-response curve plotting relative fluorescence unit (RFU) versus concentration (M) and the IC₅₀ of inhibitor compound F684 against the FEN1 protein.

FIG. 22 shows a dose-response curve plotting relative fluorescence unit (RFU) versus concentration (M) and the IC₅₀ of inhibitor compound F685 against the FEN1 protein.

FIG. 23 shows a dose-response curve plotting relative fluorescence unit (RFU) versus concentration (M) and the IC₅₀ of inhibitor compound D351 against the FEN1 protein.

FIG. 24 shows a dose-response curve plotting relative fluorescence unit (RFU) versus concentration (M) and the IC₅₀ of inhibitor compound D431 against the FEN1 protein.

FIG. 25 shows a dose-response curve plotting relative fluorescence unit (RFU) versus concentration (M) and the IC₅₀ of inhibitor compound G889 against the FEN1 protein.

FIG. 26 shows a dose-response curve plotting relative fluorescence unit (RFU) versus concentration (M) and the IC₅₀ of inhibitor compound F571 against the FEN1 protein.

FIG. 27 shows a derivative of thermal shift assay melt curve plotting the derivative of thermal shift versus temperature for inhibitor compound C200.

FIG. 28 shows a derivative of thermal shift assay melt curve plotting the derivative of thermal shift versus temperature for inhibitor compound D351.

FIG. 29 shows a derivative of thermal shift assay melt curve plotting the derivative of thermal shift versus temperature for inhibitor compound G889.

FIG. 30 shows a derivative of thermal shift assay melt curve plotting the derivative of thermal shift versus temperature for inhibitor compound F684.

FIG. 31 shows a derivative of thermal shift assay melt curve plotting the derivative of thermal shift versus temperature for inhibitor compound D413.

FIG. 32 shows a derivative of thermal shift assay melt curve plotting the derivative of thermal shift versus temperature for inhibitor compound F571.

FIG. 33 shows a derivative of thermal shift assay melt curve plotting the derivative of thermal shift versus temperature for inhibitor compound F685.

FIG. 34 shows a graph plotting relative fluorescence unit (RFU) versus time for EXO1 nuclease with varying concentrations of inhibitor compound F684.

FIG. 35 shows a graph plotting the relative activity of EXO1 versus inhibitor concentration with varying concentrations of DNA substrate and inhibitor compound F684.

FIG. 36 shows a Michaelis-Menten Graph plotting reaction rate (Vₒ) versus substrate concentration ([sub]) for inhibitor compound C200.

FIG. 37 shows a Michaelis-Menten Graph plotting reaction rate (Vₒ) versus substrate concentration ([sub]) for inhibitor compound F684.

FIG. 38 shows a Lineweaver-Burk graph plotting initial reaction velocity (1/Vₒ) versus the reciprocal of the substrate concentration (1/[S]) for inhibitor compound C200.

FIG. 39 shows a Lineweaver-Burk graph plotting initial reaction velocity (1/Vₒ) versus the reciprocal of the substrate concentration (1/[S]) for inhibitor compound F684.

FIG. 40 shows a Corey-Pauling-Koltun (CPK) model of the EXO1 protein with predicted druggable Site 1 binding pocket identified.

FIG. 41 shows a Corey-Pauling-Koltun (CPK) model of the EXO1 protein with predicted druggable Site 2 binding pocket identified.

FIG. 42 shows an image of a sodium dodecyl sulfate-polyacrylamide (SDS-PAGE) gel showing the purity of purified recombinant 6×His-tagged EXO1 N-terminal domain protein with point mutations.

FIG. 43 shows a graph plotting the total number of direct bonds between EXO1 and inhibitor compounds (interaction count) versus the residue identity for predicted binding Site 1.

FIG. 44 shows a graph plotting the total number of direct bonds between EXO1 and inhibitor compounds (interaction count) versus the residue identity for predicted binding Site 2.

FIG. 45 shows a comparison of the chemical bonds and position of inhibitor compound C200 within the EXO1 protein predicted Site 1. Left Panel shows all residues within 5 Å of inhibitor compound C200. Middle Panel shows the proximity of inhibitor compound C200 in relation to the two catalytic metal ions within the Site 1. Right Panel shows the electron density of the protein surface surrounding inhibitor compound C200.

FIG. 46 shows a comparison of the chemical bonds and position of inhibitor compound C200 within the EXO1 protein predicted Site 2. Left Panel shows all residues within 5 Å of inhibitor compound C200. Middle Panel shows the proximity of inhibitor compound C200 in relation to the two catalytic metal ions within the Site 2. Right Panel shows the electron density of the protein surface surrounding inhibitor compound C200.

FIG. 47 shows an image showing the positional comparison of inhibitor compound C200 in the EXO1 protein predicted binding Site 1 before and after EXO1-R92A and EXO1-D225A mutations.

FIG. 48 shows an image showing the positional comparison of inhibitor compound C200 in the EXO1 protein predicted binding Site 1 before and after EXO1-E89A, EXO1-K85A, and EXO1-I125A mutations.

FIG. 49 shows an image showing the positional comparison of inhibitor compound C200 in the EXO1 protein predicted binding Site 2 before and after EXO1-K292A-K294A mutation.

FIG. 50 shows a graph plotting relative fluorescence unit (FRU) versus time demonstrating the enzymatic activity of increasing concentrations of the EXO1-WT protein.

FIG. 51 shows a graph plotting relative fluorescence unit (FRU) versus time demonstrating the enzymatic activity of increasing concentrations of the EXO1 protein with a K85A mutation in the binding site.

FIG. 52 shows a graph plotting relative fluorescence unit (FRU) versus time demonstrating the enzymatic activity of increasing concentrations of the EXO1 protein with a D225A mutation in the binding site.

FIG. 53 shows a graph plotting relative fluorescence unit (FRU) versus time demonstrating the enzymatic activity of increasing concentrations of the EXO1 protein with a E89A mutation in the binding site.

FIG. 54 shows a graph plotting relative fluorescence unit (FRU) versus time demonstrating the enzymatic activity of increasing concentrations of the EXO1 protein with a K292A-K294A mutation in the binding site.

FIG. 55 shows a graph plotting relative fluorescence unit (FRU) versus time demonstrating the enzymatic activity of increasing concentrations of the EXO1 protein with a R92A mutation in the binding site.

FIG. 56 shows a graph plotting relative fluorescence unit (FRU) versus time demonstrating the enzymatic activity of increasing concentrations of the EXO1 protein with a I125A mutation in the binding site.

FIG. 57 shows a graph plotting inhibitory activity versus increasing concentration of inhibitor compound C200 in the EXO1 protein with Site 1 and Site 2 mutations.

FIG. 58 shows a graph plotting inhibitory activity versus increasing concentration of inhibitor compound F684 in the EXO1 protein with Site 1 and Site 2 mutations.

FIG. 59 shows fluorescent images of DNA double strand break burden in parental MDA-BM-436 (BRAC1−) and WT BRAC− expressing MDA-MB-436 (BRAC1+) cells after treatment with inhibitor compound C200.

FIG. 60 shows a graph that quantifies phosphorylated histone H2AX (γ-HSAX) per nucleus of parental MDA-BM-436 (BRAC1−) and WT BRAC− expressing MDA-MB-436 (BRAC1+) cells after treatment with inhibitor compound C200.

FIG. 61 shows a western blot analysis of phosphorylated histone H2AX (γ-HSAX) of parental MDA-BM-436 (BRAC1−) and WT BRAC− expressing MDA-MB-436 (BRAC1+) cells after treatment with inhibitor compound C200.

FIG. 62 shows fluorescent images of DNA double strand break burden in parental MDA-BM-436 (BRAC1−) and WT BRAC− expressing MDA-MB-436 (BRAC1+) cells after treatment with inhibitor compound F684.

FIG. 63 shows a graph that quantifies phosphorylated histone H2AX (γ-HSAX) per nucleus of parental MDA-BM-436 (BRAC1−) and WT BRAC− expressing MDA-MB-436 (BRAC1+) cells after treatment with inhibitor compound F684.

FIG. 64 shows a western blot analysis of phosphorylated histone H2AX (γ-HSAX) of parental MDA-BM-436 (BRAC1−) and WT BRAC− expressing MDA-MB-436 (BRAC1+) cells after treatment with inhibitor compound F684.

FIG. 65 shows the fluorescent images of S-phase PARylation in parental MDA-BM-436 (BRAC1−) and WT BRAC− expressing MDA-MB-436 (BRAC1+) cells after treatment with inhibitor compound C200.

FIG. 66 shows a graph that quantifies relative PARylation per nucleus of parental MDA-BM-436 (BRAC1−) and WT BRAC− expressing MDA-MB-436 (BRAC1+) cells after treatment with inhibitor compound C200.

FIG. 67 shows the fluorescent images of S-phase PARylation in parental MDA-BM-436 (BRAC1−) and WT BRAC− expressing MDA-MB-436 (BRAC1+) cells after treatment with inhibitor compound F684.

FIG. 68 shows a graph that quantifies relative PARylation per nucleus of parental MDA-BM-436 (BRAC1−) and WT BRAC− expressing MDA-MB-436 (BRAC1+) cells after treatment with inhibitor compound F684.

FIG. 69 shows EXO1 western blot analysis of inhibition by compounds C200 or F684 on impaired replication fork-related DNA end resection in MDA-MB-231 breast cancer cells at similar levels.

FIG. 70 shows a graph plotting percentage of GFP positive cells versus increasing concentration of inhibitor compound C200 and DNA2 inhibitor compound C5.

FIG. 71 shows a graph plotting percentage of GFP positive cells versus increasing concentration of inhibitor compound F684 and DNA2 inhibitor compound C5.

FIG. 72 shows an image gradient heatmap of representative inhibitor compound IC₅₀'s across 31 cancer cells lines compared to the previously reported EXO1 inhibitor, C73.

FIG. 73 shows a graph plotting Annexin V fluorescence intensity versus time for the treatment of WT BRCA1-expressing MDA-MB-436 (BRCA1+) and MDA-MB-436 parental (BRCA1−) cell lines after treatment with inhibitor compound F684.

FIG. 74 shows a graph plotting the cumulative survival percentage of MDA-MB-436 (BRCA1−) and MDA-MB-436 (BRCA1+) cells after treatment with increasing concentrations of F684.

FIG. 75 shows a graph plotting normalized tumor volumes in mouse xenograft models established using WT BRCA1 expressing MDA-MB-436 (BRCA1+) and parental MDA-MB-436 (BRCA1−) cell lines versus time after treatment with inhibitor compound F684 and vehicle (dimethyl sulfoxide—DMSO).

FIG. 76 shows a graph plotting normalized tumor volumes in mouse xenograft models established using WT BRCA1 expressing MDA-MB-436 (BRCA1−) and parental MDA-MB-436 (BRCA1−) cell lines versus time after treatment with inhibitor compound F684 and vehicle (dimethyl sulfoxide—DMSO).

FIG. 77 shows a graph plotting the ratio of P-RPA/RPA levels calculated from the western blot analysis for compound C200 in FIG. 69.

### DETAILED DESCRIPTION

A major disadvantage of conventional chemotherapy is its lack of cancer cell specificity. Therefore, identifying pathways that specifically support cancer cells is key for effective treatment. Cancer cells with activated oncogenes, aberrant checkpoints, and/or aneuploid/polyploid genomes have aberrant deoxyribonucleic acid (DNA) replication, which manifests as a high level of double-strand breaks (DSBs) due to stalling and collapse of DNA replication forks. Given that a single unrepaired DSB may cause cell death, most chemotherapies also target replication forks to directly or indirectly induce DNA DSBs. Targeting both DNA replication and DNA repair has recently been proposed as a major opportunity to enhance current cancer therapies based on DNA-damaging agents by blocking DSB repair. Recent advancements in describing the mechanisms of DNA replication in cancer cells under stress conditions suggest that aberrant DNA replication and repair processes in cancer cells are interconnected. The inventors have surprisingly and unexpectedly discovered that one way to maximally harness DSBs in cancer cells for cancer therapy may be to target the replication-repair interface that processes replication intermediates and repairs replication-associated DSBs.

Exonuclease 1 (EXO1) is a key enzyme in the replication-repair interface. EXO1 is an evolutionarily conserved member of the xeroderma pigmentosum group G (XPG)/Rad2 family of nucleases and plays a multifaceted role in DNA metabolic pathways governing genome stability, including double-strand break repair (DSBR), mismatch repair, telomere maintenance, restarting of stalled replication forks, and lagging DNA strand maturation. EXO1 has also been shown to be an important nuclease involved in Okazaki fragment maturation (OFM), the process by which RNA-DNA flaps are removed to generate ligatable nicks for the ligation of individual Okazaki fragments into the intact lagging DNA strand during replication. If the OFM machinery is inhibited, the persistent presence of flap and nick structures behind the advancing replication fork makes the cell highly susceptible to apoptosis, as significant DNA replication stress and double-strand breaks (DSBs) will occur. Blocking nucleases that are essential to the OFM process, such as EXO1, would generate persistent flap/nick substrates in the S-phase of the cell cycle and subsequently cause DSBs, particularly in cancer cells, which already bear an increased burden of replication stress. Meanwhile, EXO1 also plays a crucial role in repairing DSBs, including those from collapsed or cleaved replication forks. The primary function of EXO1 may be considered to be its role in DSB end resection to generate 3′ ssDNA overhangs, which in turn activates the master signaling kinase ATR20-22 and promotes the assembly of a RAD51-loaded nucleoprotein filament which invades the sister chromatid DNA to initiate DSB repair (DSBR) via homologous recombination (HR). As such, DNA end resection may be a critical step in DSB repair by HR. Given that cancer cells have an increased reliance on EXO1 to counteract stress due to abnormal and overactive DNA replication processes, small molecules targeting EXO1 represent a major opportunity to specifically kill cancer cells by targeting aberrant replication-associated repair systems, particularly in human breast cancer and other hormone-related cancers which bear an increased burden of replication stress due to heightened hormone signaling.

Given its dual role in DNA replication and DSB repair, EXO1 may not only be an attractive target for cancer cell-specific killing, but also has high potential for inducing synthetic lethality of cancer cells. Accordingly, the inventors reasoned that a small molecule EXO1 inhibitor may not only impair OFM, causing persistent single-strand DNA (ssDNA) breaks with the potential for conversion into DSBs, but may also simultaneously blocking DSB repair by blocking the end resection required for HR. This strategy has potential to induce synthetic lethality in human cancers with pre-existing HR gene deficiencies (HRD), e.g., BRCA1/2 mutations, as well as sensitize cancer cells to PARP inhibitors (PARPis), which are the current standard treatment for HRD cancers. In addition, it was hypothesized that EXO1 inhibitors would display greater specificity than PARPis for synthetic lethality with HRD, because PARPs participate in a wide array of other cellular processes, such as transcription and translation, whereas EXO1 does not. By itself, EXO1 may also be a crucial enzyme for HR, thus it's deficiency would also yield HRD, offering an additional opportunity for synthetic lethality with PARPi. Thus, EXO1 inhibitor-induced HRD may extend PARPi therapy to larger patient populations without HDR gene mutations.

Previously small molecules with EXO1 inhibitory activity were described, i.e., C73 and C5. See Paiano J, Zolnerowich N, Wu W, et al. Role of 53BP1 in end protection and DNA synthesis at DNA breaks. Genes Dev 2021; 35(19-20): 1356-67; Liu, Wenpeng, et al. “A selective small molecule DNA2 inhibitor for sensitization of human cancer cells to chemotherapy.” EBioMedicine 6 (2016): 73-86. Although C73 functions as an inhibitor, it has a high molecule weight, high total polar surface area, and poor solubility, which are properties considered to be predictors of reduced bioavailability.

Therefore, an unmet need for inhibitors with favorable pharmacokinetic properties remained. Accordingly, the present disclosure provides a selective and effective EXO1 inhibitor that could be used as both a research tool (i.e., chemical probe) and a pre-clinical starting point toward the development of a novel therapeutic drug that induces cancer cell-specific killing via synthetic lethality. Full length human EXO1 protein (SEQ ID NO: 1) was purified and untagged at large scale for use in a 45,000 small molecule high-throughput screen to identify novel EXO1-selective inhibitors. Compounds were screened via a robust fluorescence resonance energy transfer (FRET) based exonuclease activity assay developed and validated by the inventors. Beneficially and unexpectedly the assay resulted in seven distinct chemical scaffolds with EXO1-selective inhibitory activity that synergized with BRCA1-deficiency in human cancer cells. In one aspect, the present disclosure describes the discovery and mode of action of novel EXO1-targeted small molecule inhibitors.

In an aspect, recombinant full-length human EXO1 protein was used to perform a FRET-based high-throughput assay to screen approximately 45,000 NCI library-deposited small molecules to identify EXO1 inhibitors, the hits of which were cheminformatically grouped into seven distinct scaffolds. In an aspect of this disclosure, the IC50s of the most potent representative compound from each of the seven inhibitor scaffolds, C200, F684, F685, D351, D413, G889, and F571, were calculated using our cell-free FRET-based exonuclease assay system and across 31 cancer cell lines of varied origin via CellTiter-Glo®. In another aspect, it was demonstrated using the most potent compound identified from each of these methods, C200 and F684, respectively, that these compounds inhibit the exonuclease activity of EXO1 biochemically and in breast cancer cells. Additionally, in a further aspect of this disclosure it was shown that compound F684 effectively reduced the growth of BRCA1− tumors in mice, suggesting this scaffold may be amenable for further optimization.

A wild-type (WT) EXO1 N-terminal domain protein structure (PDB 3QEB; aa 1-352) was used to identify the two binding pockets, designated as Sites 1 and 2, where drug-like molecules can preferentially dock. Site 1 is a well-defined pocket located in the active site of the globular N-terminal domain, whereas Site 2 is a shallow allosteric surface pocket located on the proximal side of the intrinsically disordered C-terminus. This in-house virtual screening may be analogous to the previous method used by the inventors to identify DNA2 inhibitors. Rigid and induced-fit docking methodologies were used to predict the preferred binding site (Site 1 vs Site 2) and binding poses of the seven inhibitor scaffolds and elucidate the EXO1 residue interactions that drive their inhibitory activity.

The N-terminal domain of EXO1 forms a well-defined bean-shaped core with a helical protrusion and numerous surface grooves. The structure can be divided into four regions, which are defined in reference to the nick at which the enzyme binds: 1) the pre-nick DNA-binding region, which holds the double-stranded segment with the substrate strand that is cleaved; 2) the post-nick binding region, which in the protein binds the single-stranded gap; 3) the active site region, which contains a metal center at which DNA cleavage takes place; and 4) a C-terminal segment. Modeled docking and biochemical data suggest that compound C200 may bind to both the metal center within active site region (R92 and D225), and the C-terminal segment (K292 and K294). This may occur because the molecular weight of compound C200 (<300 g/mol) may be small enough to be considered a “fragment hit”, thus it may bind to many sites in the EXO1 protein necessary to activate the nuclease. This rationale may be consistent with the activity of compound C200 in double strand break (DSB), homogeneous recombination (HR), and cytotoxicity experiments compared to the florescence resonance energy transfer (FRET)-based exonuclease assay that used for high-throughput screening (HTS). Additionally, the structure of compound C200 is similar to thymine, which could mean compound C200 may also interact with the DNA substrate—a finding that may be consistent with the observation that compound C200 has a stabilizing effect on EXO1, as indicated by a ΔTm of 1.25 °C in our thermal shift binding assay. In contrast, while the docking model described herein suggests that F684 may also bind to either Site 1 or Site 2, at a relatively low concentration of 10 pM its activity appears to be influenced by interactions with R92 and K85 within the active site; however, at concentrations above 10 μM, its interaction with Site 2 likely overcomes this influence by inducing a conformational change that directly/indirectly blocks the nucleolytic activity of EXO1. Together, these findings suggest the requirement of both the active site and C-terminal domains for EXO1 nuclease activity. Furthermore, while the active site has been extensively characterized, the distinct mechanisms of the described EXO1 inhibitors may provide a new opportunity to study the helical protrusion and surface grooves, the function of which remain largely conjectural. X-ray crystallography studies to reveal the precise binding relationship between EXO1 and compounds C200 and F684 and provide more definitive insight into the mechanisms hypothesized for these compounds.

In some aspects, a series of functional analyses were used to characterized the specific functions of EXO1 that compounds C200 and F684 inhibit. Beneficially and unexpectedly it was discovered that C200 and F684 suppress end resection at CPT-stalled DNA replication forks, as indicated by a decrease in phospho-RPA after treatment, and this effect was more pronounced when treatment was combined with the DNA2 inhibitor, C5. Additionally, F684 may be capable of selectively inducing replication-coupled DSBs in parental MDA-MB-436 (BRCA1−) cells and mouse xenograft models. Furthermore, both as a single agent and in combination with C5, compound F684 caused a significant reduction in homologous recombination. Surprisingly and beneficially, it was found that EXO1 inhibitors can both induce HRD and elicit synthetic lethality in the context of pre-existing genetic defects in HR-related proteins, such as breast cancer gene 1 (BRCA1). Additionally, congruent with biochemical data, it was also found that F684 also showed strong killing effects in cancer cell lines.

In an aspect, the present disclosure provides additional support to existing evidence that suggests a major function of EXO1 is to participate in the protection, remodeling, and restart of stalled replication forks. BRCA1 is known to contribute to the expansion of ssDNA gaps, and it is hypothesized that EXO1 becomes essential in BRCA1-deficient contexts. This dependence on DNA end resection may highlight a potential vulnerability of BRCA1-mutant tumors to EXO1 inhibition. The present disclosure provides support for the notion that EXO1 inhibitors generate poly(ADP-ribose)-decorated DNA lesions during S-phase that are associated with unresolved DSBs and genomic instability in BRCA1-deficient but not in WT BRCA1 cells. PARP inhibitors that were developed for the treatment of cancers with HRD, such as BRCA1− and BRCA2-defective cancers, are a highly successful example of synthetic lethality. Deficiency in any of the homology-directed repair genes has been found to lead to defective DSB repair, thereby promoting PARPi synthetic lethality. Our finding that EXO1 inhibitors can independently induce HRD suggests that they may be effective in both sensitizing cells to PARPi and overcoming resistance to them.

Given that cancer cells require EXO1 to counteract the immense stress of abnormal DNA replication, small molecules targeting EXO1 exonuclease activity represent a major opportunity for specifically killing cancer cells. Because it is not an essential protein under normal circumstances, EXO1 inhibition would effectively target the defective replication-associated DSB repair systems found in many cancer types, particularly those that are either hormone-dependent, already possess mutations in HR proteins like BRCA1/2, or have acquired resistance to other DSB response-targeting agents such as PARPi.

In some aspects, the present disclosure tested if targeting EXO1 may exploit a specific vulnerability in cancer cells, assuming that normal cells are better protected by intact cell cycle checkpoints and redundant DNA repair processes. It may be suggested that EXO1 inhibitors could be possible therapeutic agents for treating cancers that are defective for HR and other DNA repair and DNA damage checkpoint pathways. Though the cytotoxicity of the present compounds was limited, suitable compounds could be identified using the described FRET-based exonuclease activity assay and further developed by subjecting them to the other assays introduced in the present EXO1 inhibitor discovery pipeline. The observation that normal cells can recover from EXO1 inhibition, whereas sensitive cells such as BRCA1-deficient cannot, suggests that cycles of inhibitor treatment might be an effective approach for the clinical use of EXO1 inhibitors in future studies.

### ABBREVIATIONS

C200: Compound I, methyl 5,6-dihydroxy-2-(4-methoxyphenyl)pyrimidine-4-carboxylate
F684: Compound II, 5-(4-(2,3-dimethylphenyl)piperazin-1-yl)-2-(phenylsulfonamido)benzoic acid
F685: Compound III, 5-(4-benzylpiperidin-1-yl)-2-(2-chlorobenzamido)benzoic acid
D351: Compound IV, 2-(4-(tert-butyl)phenyl)-5-(((3-chloro-2-methylphenyl)amino)methyl)-[1,2,4]triazolo[1,5-a]pyrimidin-7(4H)-one
D413: Compound V, 4-chloro-N-(5-(4-chlorophenyl)-7-phenyl-4,7-dihydro-[1,2,4]triazolo[1,5-a]pyrimidin-2-yl)benzenesulfonamide
G889: Compound VI, 2-(4-(2-chlorophenyl)piperazin-1-yl)-5-(2-methylbenzamido)benzoic acid
F571: Compound VII, 2,5-dimethylphenyl 2-((2-(4-chlorophenyl)-7-oxo-6,7-dihydropyrazolo[1,5-a][1,3,5]triazin-4-yl)thio)acetate

### DEFINITIONS

While various some aspects and aspects of the present invention are shown and described herein, such some aspects and aspects are provided by way of example only. Numerous variations, changes, and substitutions will now occur to those skilled in the art without departing from the invention. It should be understood that various alternatives to some aspects described herein may be employed.

Before the present disclosure is described in greater detail, it is to be understood that this disclosure is not limited to particular examples described, and as such may, of course, vary. It is also to be understood that the terminology used herein is for the purpose of describing particular examples only, and is not intended to be limiting, since the scope of the present disclosure will be limited only by the appended claims.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 3nd ed., J. Wiley & Sons (New York, NY 2006); Sambrook et al., MOLECULAR CLONING, A LABORATORY MANUAL, 4^{th} ed., Cold Springs Harbor Press (Cold Springs Harbor, NY 2014). Any methods, devices and materials similar or equivalent to those described herein can be used. The following definitions are provided to facilitate understanding of certain terms used frequently herein and are not meant to limit the scope of the present disclosure.

The term “fluorescence” refers to the phenomenon where a compound absorbs light at one wavelength and then emits light at a longer wavelength, i.e., it absorbs light of one color and emits light of a different color. The emission is due to the elections in the compound being excited to a higher energy state and then releasing that energy as light when returning to their ground state, a process that may happen quickly and only while the excitation light is present.

The term “patient” or “subject” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some aspects, a patient is human.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

The term “administering” as used herein means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds can be administered alone or can be co-administered to a subject. Co-administration is meant to include simultaneous or sequential administration of the compounds individually or in combination (more than one compound). Thus, the preparations 20 5 10 15 20 25 30 can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In some aspects, the formulations of the compositions of the present invention can be delivered using liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Cnrr. Opin. Biotechnol. 6:698-708, 1995; Ostro, Am. J. EIosp. Pharm. 46: 1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

Compounds are described using standard nomenclature. Unless defined otherwise, all technical and scientific terms used herein have the same meaning as is commonly understood by one of skill in the art to which the invention belongs.

The terms “a” and “an” do not denote a limitation of quantity, but rather denote the presence of at least one of the referenced item. The term “or” means “and/of”. The term “and/or” as used herein means that the listed items are present, or used, individually or in combination. In effect, this term means that “at least one of” or “one or more” of the listed items is used or present.

As used herein, the terms “optional” or “optionally” mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

As used herein, the term “each,” when used in reference to a collection of items, is intended to identify an individual item in the collection but does not necessarily refer to every item in the collection. Exceptions can occur if explicit disclosure or context clearly dictates otherwise.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used herein, a dash (“-”) that is not between two letters or symbols is used to indicate a point of attachment for a substituent. For example, —(C═O)NH2 is attached through the carbon of the keto (C═O) group.

As used herein, “analog” or “analogue” is used in accordance with its plain ordinary meaning within Chemistry and Biology and refers to a chemical compound that may be structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog may be a similar compound or a compound comparable in function and appearance but not in structure or origin to a reference compound.

Certain compounds of the present invention may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)-, and individual isomers are encompassed within the scope of the present disclosure. The compounds of the present disclosure do not include those that are known in art to be too unstable to synthesize and/or isolate. The present disclosure may include compounds in racemic and optically pure forms. Optically active (R)- and (S)-isomers may be prepared using chiral synthons or chiral reagents or resolved using conventional techniques.

As used herein, the term “isomers” refers to compounds having the same number and kind of atoms, and hence the same molecular weight, but differing in respect to the structural arrangement or configuration of the atoms.

As used herein, the term “tautomer” refers to one of two or more structural isomers which exist in equilibrium, and which are readily converted from one isomeric form to another.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present disclosure can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Description of compounds of the present disclosure may be limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Where substituent groups are specified by their conventional chemical formulae, written from left to right, they equally encompass the chemically identical substituents that would result from writing the structure from right to left, e.g., —CH₂O— is equivalent to —OCH₂—.

As used herein, “alkyl” is a branched or straight chain saturated aliphatic hydrocarbon group. In some cases, the alkyl group contains from 1 to about 30 carbon atoms, more generally from 1 to about 20 carbon atoms, or from 1 to about 12 carbon atoms, or from 1 to about 8 carbon atoms. In some cases, the alkyl contains from 1 to about 20 carbon atoms. In some cases, the alkyl is C1-C2, C1-C3, C1-C4, C1-C5, C1-C6, C1-C7, C1-C8, C1-C9, C1-C10, C1-C11, C1-C12, C1-C13, C1-C14, C1-C15, C1-C16, C1-C17, C1-C18, C1-C19, C1-C20, C1-C21, C1-C22, C1-C23, C1-C24, C1-C25, C1-C26, C1-C27, C1-C28, C1-C29, or C1-C30. The specified ranges as used herein indicate an alkyl group having each member of the range described as an independent species. For example, the term C1-C20 alkyl as used herein indicates a straight or branched alkyl group having from 1, 2, 3, 4, 5, 6, 7, 8, 9, 10, 11, 12, 13, 14, 15, 16, 17, 18, 19, or 20 carbon atoms and is intended to mean that each of these is described as an independent species. For example, the term C1-C4 alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In some cases, the alkyl group is optionally substituted. The term “alkyl” also encompasses cycloalkyl or carbocyclic groups. For example, when a term is used that includes “alk” then “cycloalkyl” or “carbocyclic” can be considered part of the definition, unless unambiguously excluded by the context. For example, and without limitation, the terms alkyl, alkoxy, etc. can all be considered to include the cyclic forms of alkyl, unless unambiguously excluded by context.

As used herein, “alkenyl” is a linear or branched aliphatic hydrocarbon groups having one or more carbon-carbon double bonds that may occur at a stable point along the chain. In some cases, “alkenyl” is used to indicate those alkenyl groups having 2-20 carbons. The specified ranges as used herein indicate an alkenyl group having each member of the range described as an independent species, as described above for the alkyl moiety. Examples of alkenyl radicals include, but are not limited to ethenyl, propenyl, allyl, propenyl, butenyl and 4-methylbutenyl. The term “alkenyl” also embodies “cis” and “trans” alkenyl geometry, or alternatively, “E” and “Z” alkenyl geometry. In an alternative embodiment, the alkenyl group is optionally substituted. The term “Alkenyl” also encompasses cycloalkyl or carbocyclic groups possessing at least one point of unsaturation.

As used herein, “alkynyl” is a branched or straight chain aliphatic hydrocarbon group having one or more carbon-carbon triple bonds that may occur at any stable point along the chain. In some cases, “alkynyl” is used to indicate those alkenyl groups having 2-20 carbons. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. In some cases, the alkynyl group is optionally substituted. The term “alkynyl” also encompasses cycloalkyl or carbocyclic groups possessing at least one point of unsaturation.

As used herein, “alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—).

As used herein, “alkanoyl” and “acyl” are used interchangeably. As used herein, “acyl” means, unless otherwise stated, —C(O)R where R is an alkyl group as defined above. For example, R may be a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl. The carbonyl carbon is included in the number of carbons, that is C2alkanoyl is a —(C═O)CH₃ group.

As used herein, “aliphatic” refers to a saturated or unsaturated, straight, branched, or cyclic hydrocarbon. “Aliphatic” is intended herein to include, but is not limited to, alkyl, alkenyl, alkynyl, cycloalkyl, cycloalkenyl, and cycloalkynyl moieties, and thus incorporates each of these definitions. In some cases, “aliphatic” is used to indicate those aliphatic groups having 1-30 carbon atoms. The aliphatic group can be for example, alkyl, mono-unsaturated, di-unsaturated, tri-unsaturated, or polyunsaturated, or alkynyl. Unsaturated aliphatic groups can be in a cis or trans configuration. In some cases, the aliphatic group contains from 1 to about 20 carbon atoms, more generally from 1 to about 12 carbon atoms or from 1 to about 8 carbon atoms. In one embodiment, the aliphatic group contains from 1 to about 20 carbon atoms. In certain embodiments, the aliphatic group is C1-C2, C1-C3, C1-C4, C1-C5, C1-C6, C1-C7, C1-C8, C1-C9, C1-C10, C1-C11, C1-C12, C1-C13, C1-C14, C1-C15, C1-C16, C1-C17, C1-C18, C1-C19, C1-C20, C1-C21, C1-C22, C1-C23, C1-C24, C1-C25, C1-C26, C1-C27, C1-C28, C1-C29, or C1-C30. The specified ranges as used herein indicate an aliphatic group having each member of the range described as an independent species. For example, the term C1-C20 aliphatic as used herein indicates a straight or branched alkyl, alkenyl, or alkynyl group having 1, 2, 3, 4, 5, 6, 7, 8, 9, 10, 11, 12, 13, 14, 15, 16, 17, 18, 19, or 20 carbon atoms and is intended to mean that each of these is described as an independent species. For example, the term C1-C4 aliphatic as used herein indicates a straight or branched alkyl, alkenyl, or alkynyl group having from 1, 2, 3, or 4 carbon atoms and is intended to mean that each of these is described as an independent species.

As used herein, “carbocyclyl”, “carbocyclic”, “carbocycle” or “cycloalkyl” is a saturated or partially unsaturated (i.e., not aromatic) group containing all carbon ring atoms and from 3 to 14 ring carbon atoms (“C3-14 cycloalkyl”) and zero heteroatoms in the non-aromatic ring system. In some cases, a cycloalkyl group has 3 to 10 ring carbon atoms (“C3-10 cycloalkyl”). In some cases, a cycloalkyl group has 3 to 9 ring carbon atoms (“C3-9 cycloalkyl”). In some cases, a cycloalkyl group has 3 to 8 ring carbon atoms (“C3-8 cycloalkyl”). In some cases, a cycloalkyl group has 3 to 7 ring carbon atoms (“C3-7 cycloalkyl”). In some cases, a cycloalkyl group has 3 to 6 ring carbon atoms (“C3-6 cycloalkyl”). In some cases, a cycloalkyl group has 4 to 6 ring carbon atoms (“C4-6 cycloalkyl”). In some cases, a cycloalkyl group has 5 to 6 ring carbon atoms (“C5-6 cycloalkyl”). In some cases, a cycloalkyl group has 5 to 10 ring carbon atoms (“C5-10 cycloalkyl”). Exemplary C3-6 cycloalkyl groups include, without limitation, cyclopropyl (C3), cyclopropenyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-8 cycloalkyl groups include, without limitation, the aforementioned C3-6 cycloalkyl groups as well as cycloheptyl (C7), cycloheptenyl (C7), cycloheptadienyl (C7), cycloheptatrienyl (C7), cyclooctyl (C8), cyclooctenyl (C8), and the like. Exemplary C3-10 cycloalkyl groups include, without limitation, the aforementioned C3-8 cycloalkyl groups as well as cyclononyl (C9), cyclononenyl (C9), cyclodecyl (C10), cyclodecenyl (C10), and the like. As the foregoing examples illustrate, in certain embodiments, the cycloalkyl group can be saturated or can contain one or more carbon-carbon double or triple bonds. In some cases, “cycloalkyl” also includes ring systems wherein the cycloalkyl ring, as defined above, is fused with one heterocycle, aryl or heteroaryl ring wherein the point of attachment is on the cycloalkyl ring, and in such instances, the number of carbons continue to designate the number of carbons in the carbocyclic ring system. In some cases, each instance of cycloalkyl is optionally substituted with one or more substituents. In some cases, the cycloalkyl group is an unsubstituted C3-14 cycloalkyl.

As used herein, the term “aryl” means, unless otherwise stated, a polyunsaturated, aromatic, hydrocarbon substituent, which can be a single ring or multiple rings (for example from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Example substituents for each of the above noted aryl and heteroaryl ring systems are described below.

As used herein, the terms “heteroatom” or “ring heteroatom” means, unless otherwise stated, atoms other than carbon or hydrogen.

As used herein, the terms “heteroalkyl,” “heterocycloalkyl,” “heteroalkenyl,” “heterocycloalkenyl,” or “heteroaryl” unless otherwise stated refer to the inclusion of a heteroatom in alkyl, cycloalkyl, alkenyl, cycloalkenyl or aryl as described herein.

As used herein the terms “cycloalkyl,” “heterocycloalkyl,” “cycloalkenyl,” “heterocycloalkenyl,” unless otherwise stated refer to cyclic versions of the alkyl, heteroalkyl, alkenyl, and heteroalkenyl as described herein.

Each of the above terms (e.g., “alkyl,” “cycloalkyl,” “heteroalkyl,” “heterocycloalkyl,” “alkenyl,” “heteroalkenyl,” “cycloalkenyl,” “heterocycloalkenyl,” “aryl,” and “heteroaryl”) includes both substituted and unsubstituted forms of the indicated group.

In some cases, each substituted group described in the compounds herein may be substituted with at least one substituent group. More specifically, in some cases, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group.

Substituents for the alkyl and heteroalkyl groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —NR′SO₂R″, —NR′C═(O)R″, —NR′C(O)—OR″, or —NR′OR″, R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkyl, alkoxy, or thioalkoxy groups, or arylalkyl groups. When a compound of the disclosure includes more than one R group, for example, each of the R groups may be independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as acyl (e.g., —C(O)CH₃, —C(O)CH₂OCH₃, and the like).

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency). When a substituent is attached to a ring, but not a specific atom (a floating substituent), and a subscript for the substituent is an integer greater than one, the multiple substituents may be on the same atom, same ring, different atoms, and each substituent may optionally be different. Where a point of attachment of a ring to the remainder of a molecule is not limited to a single atom (a floating substituent), the attachment point may be any atom of the ring while obeying the rules of chemical valency. Where a ring contains one or more ring heteroatoms and the ring, is shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

In some cases, a “substituent group,” as used herein, means a group selected from the following moieties: —CN, —OH, —NH₂, —COOH, —CONH₂, —NO₂, —SH, —SCH₃, —SO₃H, —SO₄H, SO₂NH₂, —NHNH₂, —ONH₂, —NHC(O)NIINH₂, —NHC(O)NH₂, —NHSO₂H, —NHC(O)H, —NHC(O)OH, —NHOH, —O-alkyl, —O-heteroalkyl, —O-cycloalkyl, —O-heterocycloalkyl, —O-aryl, —O-heteroaryl, —O-alkenyl, —O-heteroalkenyl, —O-heterocycloalkenyl, substituted or unsubstituted alkyl, substituted or unsubstituted heteroalkyl, substituted or unsubstituted cycloalkyl, substituted or unsubstituted heterocycloalkyl, substituted or unsubstituted alkenyl, substituted or unsubstituted heteroalkenyl, substituted or unsubstituted heterocycloalkenyl, substituted or unsubstituted aryl, and/or substituted or unsubstituted heteroaryl.

### Inhibitor Compounds

The present disclosure provides human exonuclease 1 inhibitor compounds of the formula (I), Formula (II), and Formula (III) or a pharmaceutically acceptable salt thereof.

In some cases, the human exonuclease 1 inhibitor compound described herein may be of Formula (I):

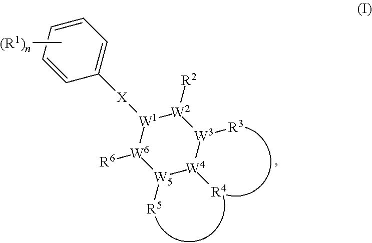

or a pharmaceutically acceptable salt thereof;
wherein X is a bond, —NH—, or —NH— alkyl-;
R¹ is absent, H, halo, or L;
R² absent, H, halo, or L;
R³ is H, halo, —OH, alkyl, bound to R⁴, or L;
R⁴ is H, halo, —OH, alkyl-L, bound to R³, bound to R⁵, or L;
R⁵ is absent, H, ═O, —C(O)O-alkyl, bound to R⁴, or L;
R⁶ is absent, H, or L;
W² is —CH—, —N═, —NH—;
W³ is —CH—, —C═;
W⁴ is —N—, —CH—, —C═;
W⁵ is —CH—, —C—, —C═;
W⁶ is —CH—, —C═, —N═;
L is ═O, —C(O)OH, alkyl, aryl, benzyl, —O-alkyl, —NHC(O)Y, —NHS(O)₂Y, SCH₂C(O)O—Y;
Y is substituted or unsubstituted aryl, substituted or unsubstituted heteroaryl, substituted or unsubstituted heterocycle,
when R³ is bound to R⁴ or R⁴ is bound to R⁵ a five membered unsubstituted or L-substituted heterocycle is formed; and
n is 0-5.

In some cases, R¹ is absent. In some cases, R¹ is H. In some cases, R¹ is halo. In some cases, R¹ is L.

In some cases, R² is absent. In some cases, R² is H. In some cases, R² is halo. In some cases, R² is L.

In some cases, R³ is H. In some cases, R³ is halo. In some cases, R³ is —OH. In some cases, R³ is alkyl. In some cases, R³ is substituted or unsubstituted C₁-C₂₀ alkyl. In some cases, R³ is substituted or unsubstituted C₁-C₂ alkyl, C₁-C₃ alkyl, C₁-C₄ alkyl, C₁-C₅ alkyl, C₁-C₆ alkyl, C₁-C₇ alkyl, C₁-C₈ alkyl, C₁-C₉ alkyl, C₁-C₁₀ alkyl, C₁-C₁₁ alkyl, C₁-C₁₂ alkyl, C₁-C₁₃ alkyl, C₁-C₁₄ alkyl, C₁-C₁₅ alkyl, C₁-C₁₆, alkyl, C₁-C₁₇ alkyl, C₁-C₁₈ alkyl, C₁-C₁₉ alkyl, or C₁-C₂₀ alkyl. In some cases, substituted or unsubstituted C₂-C₃ alkyl, C₂-C₄ alkyl, C₂-C₅ alkyl, C₂-C₆ alkyl, C₂-C₇ alkyl, C₂-C₈ alkyl, C₂-C₉ alkyl, C₂-C₁₀ alkyl, C₂-C₁₁ alkyl, C₂-C₁₂ alkyl, C₂-C₁₃ alkyl, C₂-C₁₄ alkyl, C₂-C₁₅ alkyl, C₂-C₁₆ alkyl, C₂-C₁₇ alkyl, C₂-C₁₈ alkyl, C₂-C₁₉ alkyl, or C₂-C₂₀ alkyl. In some cases, R³ is substituted or unsubstituted C₃-C₄ alkyl, C₃-C₅ alkyl, C₃-C₆ alkyl, C₃-C₇ alkyl, C₃-C₈ alkyl, C₃-C₉ alkyl, C₃-C₁₀ alkyl, C₃-C₁₁ alkyl, C₃-C₁₂ alkyl, C₃-C₁₃ alkyl, C₃-C₁₄ alkyl, C₃-C₁₅ alkyl, C₃-C₁₆ alkyl, C₃-C₁₇ alkyl, C₃-C₁₈ alkyl, C₃-C₁₉ alkyl, or C₃-C₂₀ alkyl. In some cases, R³ is substituted or unsubstituted C₄-C₅ alkyl, C₄-C₆ alkyl, C₄-C₇ alkyl, C₄-C₈ alkyl, C₄-C₉ alkyl, C₄-C₁₀ alkyl, C₄-C₁₁ alkyl, C₄-C₁₂ alkyl, C₄-C₁₃ alkyl, C₄-C₁₄ alkyl, C₄-C₁₅ alkyl, C₄-C₁₆ alkyl, C₄-C₁₇ alkyl, C₄-C₁₈ alkyl, C₄-C₁₉ alkyl, or C₄-C₂₀ alkyl. In some cases, R³ is bound to R⁴. In some cases, R³ is L.

In some cases, R⁴ is H. In some cases, R⁴ is halo. In some cases, R⁴ is —OH. In some cases, R⁴ is alkyl-L. In some cases, R⁴ is substituted or unsubstituted C₁-C₂₀ alkyl. In some cases, R² is substituted or unsubstituted C₁-C₂ alkyl-L, C₁-C₃ alkyl-L, C₁-C₄ alkyl-L, C₁-C₅ alkyl-L, C₁-C₆ alkyl-L, C₁-C₇ alkyl-L, C₁-C₈ alkyl-L, C₁-C₉ alkyl-L, C₁-C₁₀ alkyl-L, C₁-C₁₁ alkyl-L, C₁-C₁₂ alkyl-L, C₁-C₁₃ alkyl-L, C₁-C₁₄ alkyl-L, C₁-C₁₅ alkyl-L, C₁-C₁₆, alkyl-L, C₁-C₁₇ alkyl-L, C₁-C₁₈ alkyl-L, C₁-C₁₉ alkyl-L, or C₁-C₂₀ alkyl-L. In some cases, R⁴ is substituted or unsubstituted C₂-C₃ alkyl-L, C₂-C₄ alkyl-L, C₂-C₅ alkyl-L, C₂-C₆ alkyl-L, C₂-C₇ alkyl-L, C₂-C₈ alkyl-L, C₂-C₉ alkyl-L, C₂-C₁₀ alkyl-L, C₂-C₁₁ alkyl-L, C₂-C₁₂ alkyl-L, C₂-C₁₃ alkyl-L, C₂-C₁₄ alkyl-L, C₂-C₁₅ alkyl-L, C₂-C₁₆ alkyl-L, C₂-C₁₇ alkyl-L, C₂-C₁₈ alkyl-L, C₂-C₁₉ alkyl-L, or C₂-C₂₀ alkyl-L. In some cases, R⁴ is substituted or unsubstituted C₃-C₄ alkyl-L, C₃-C₅ alkyl-L, C₃-C₆ alkyl-L, C₃-C₇ alkyl-L, C₃-C₈ alkyl-L, C₃-C₉ alkyl-L, C₃-C₁₀ alkyl-L, C₃-C₁₁ alkyl-L, C₃-C₁₂ alkyl-L, C₃-C₁₃ alkyl-L, C₃-C₁₄ alkyl-L, C₃-C₁₅ alkyl-L, C₃-C₁₆ alkyl-L, C₃-C₁₇ alkyl-L, C₃-C₁₈ alkyl-L, C₃-C₁₉ alkyl-L, or C₃-C₂₀ alkyl-L. In some cases, R⁴ is substituted or unsubstituted C₄-C₅ alkyl-L, C₄-C₆ alkyl-L, C₄-C₇ alkyl-L, C₄-C₈ alkyl-L, C₄-C₉ alkyl-L, C₄-C₁₀ alkyl-L, C₄-C₁₁ alkyl-L, C₄-C₁₂ alkyl-L, C₄-C₁₃ alkyl-L, C₄-C₁₄ alkyl-L, C₄-C₁₅ alkyl-L, C₄-C₁₆ alkyl-L, C₄-C₁₇ alkyl-L, C₄-C₁₈ alkyl-L, C₄-C₁₉ alkyl-L, or C₄-C₂₀ alkyl-L. In some cases, R⁴ is bound to R³. In some cases, R⁴ is bound to R⁵. In some cases, R⁴ is L.

In some cases, R⁵ is absent. In some cases, R⁵ is H. In some cases, R⁵ is ═O. In some cases, R⁵ is —C(O)O-alkyl. In some cases, R⁵ is bound to R⁴. In some cases, R⁵ is L.

In some cases, R⁶ is absent. In some cases, R⁶ is H. In some cases, R⁶ is L.

In some cases, W² is —CH—. In some cases, W² is —N═. In some cases, W² is —NH—.

In some cases, W³ is —CH—. In some cases, W³ is —C═.

In some cases, W⁴ is —N—. In some cases, W⁴ is —CH—. In some cases, W⁴ is —C═.

In some cases, W⁵ is —CH—. In some cases, W⁵ is —C—. In some cases, W⁵ is —C═.

In some cases, W⁶ is —CH—. In some cases, W⁶ is —C═. In some cases, W⁶ is —N═.

In some cases, L is ═O. In some cases, L is —C(O)OH. In some cases, L is alkyl. In some cases, L is aryl. In some cases, L is benzyl. In some cases, L is —O-alkyl. In some cases, L is —NHC(O)Y. In some cases, L is —NHS(O)₂Y. In some cases, L is SCH₂C(O)O—Y.

In some cases, Y is substituted or unsubstituted aryl, substituted or unsubstituted heteroaryl, substituted or unsubstituted heterocycle,

In some cases, when R³ is bound to R⁴ or R⁴ is bound to R⁵ a five membered unsubstituted or L-substituted heterocycle is formed. In some cases a unsubstituted or L-substituted pyrrole is formed. In some cases a unsubstituted or L-substituted furan is formed. In some cases a unsubstituted or L-substituted thiophene is formed. In some cases a unsubstituted or L-substituted pyrazole is formed. In some cases a unsubstituted or L-substituted imidazole is formed. In some cases a unsubstituted or L-substituted oxazole is formed. In some cases a unsubstituted or L-substituted thiazole is formed. In some cases a unsubstituted or L-substituted triazole is formed. In some cases a unsubstituted or L-substituted tetrazole is formed.

In some cases, n is 0-5.

In some cases, the human exonuclease 1 inhibitor has the structure of Formula (II):

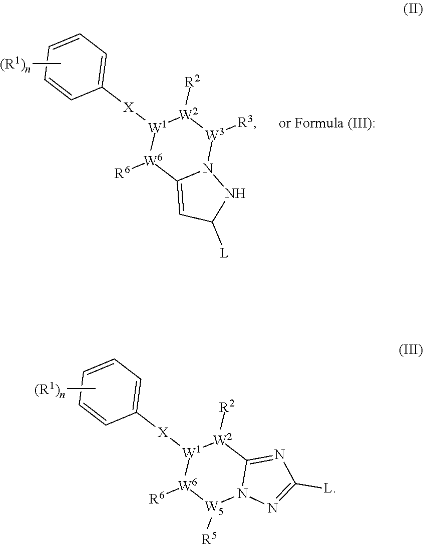

In some cases, L is:

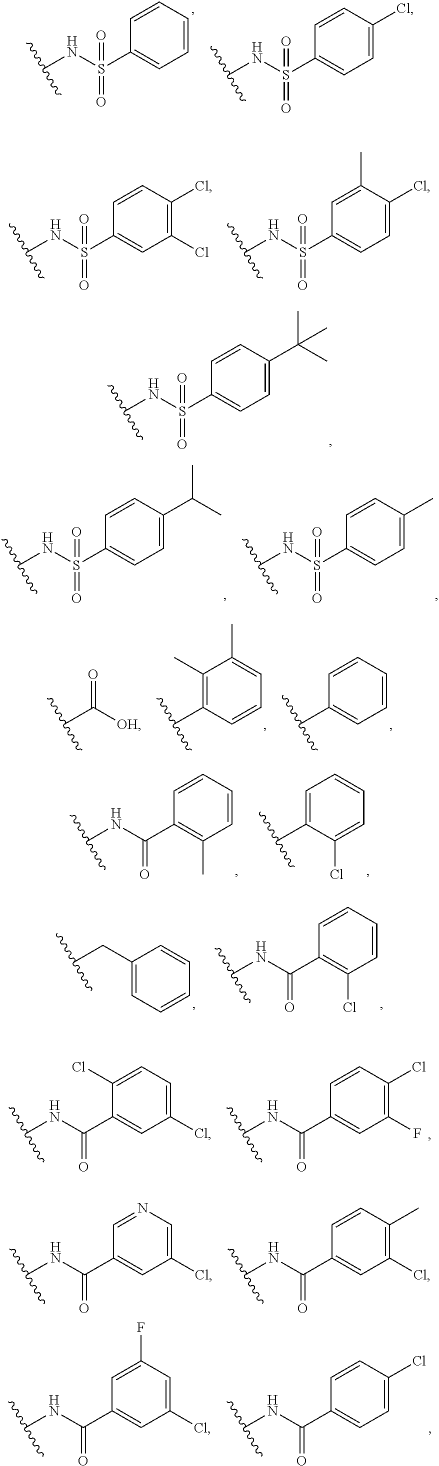

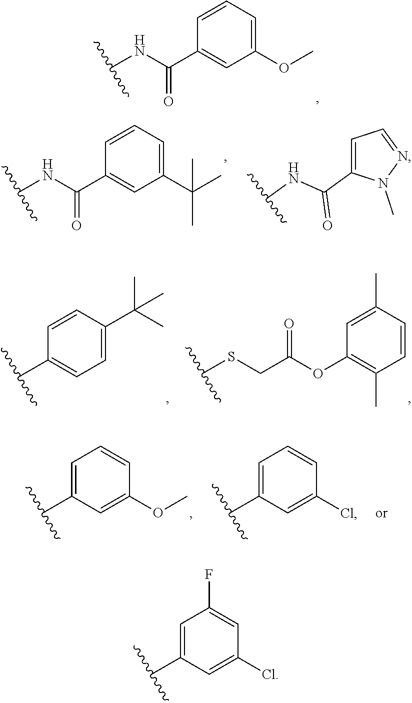

In some cases, Y is:

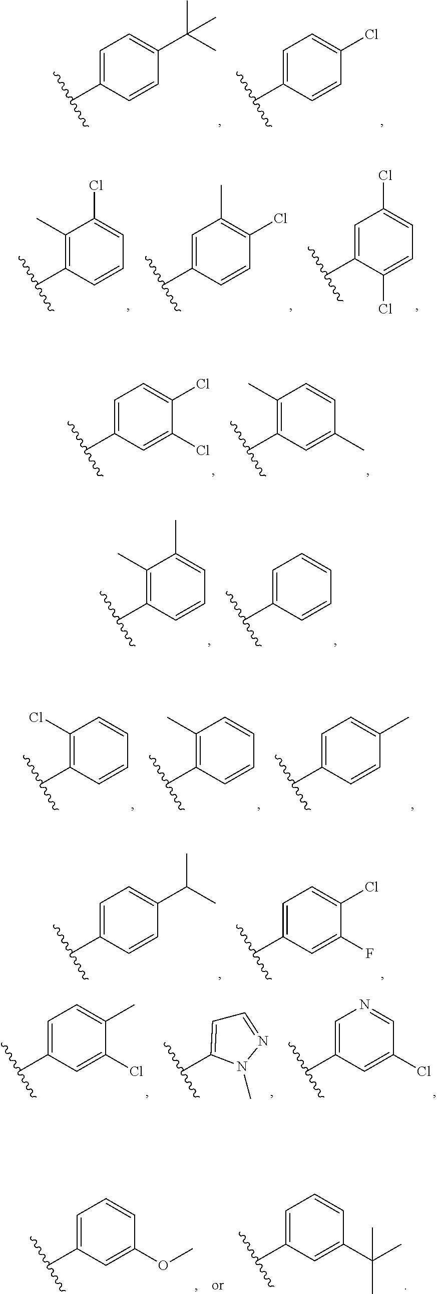

In some cases the exonuclease 1 inhibitor compound is:

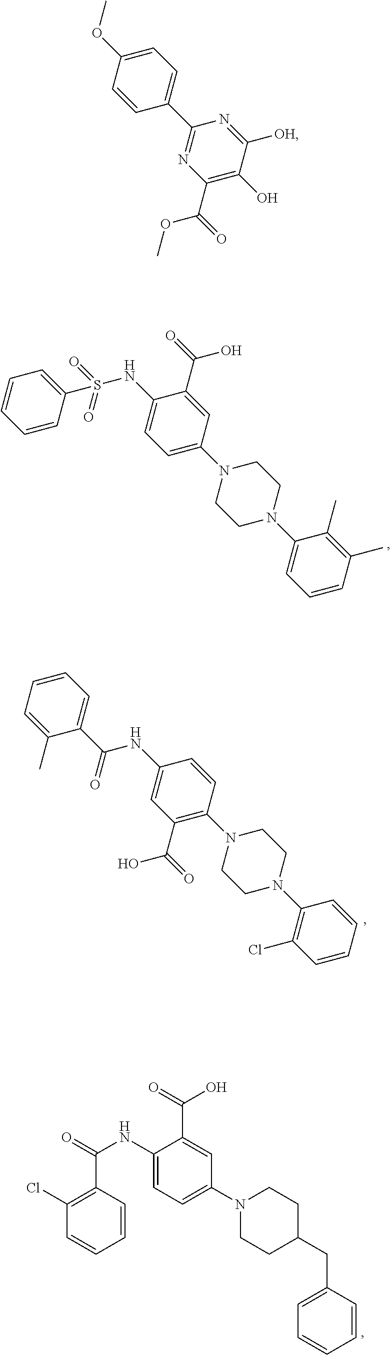 [SMILES: COC(=O)c1nc(-c2ccc(OC)cc2)nc(O)c1O ; Cc1cccc(N2CCN(c3ccc(NS(=O)(=O)c4ccccc4)c(C(=O)O)c3)CC2)c1C ; Cc1ccccc1C(=O)Nc1ccc(N2CCN(c3ccccc3Cl)CC2)c(C(=O)O)c1 ; O=C(Nc1ccc(N2CCC(Cc3ccccc3)CC2)cc1C(=O)O)c1ccccc1Cl]

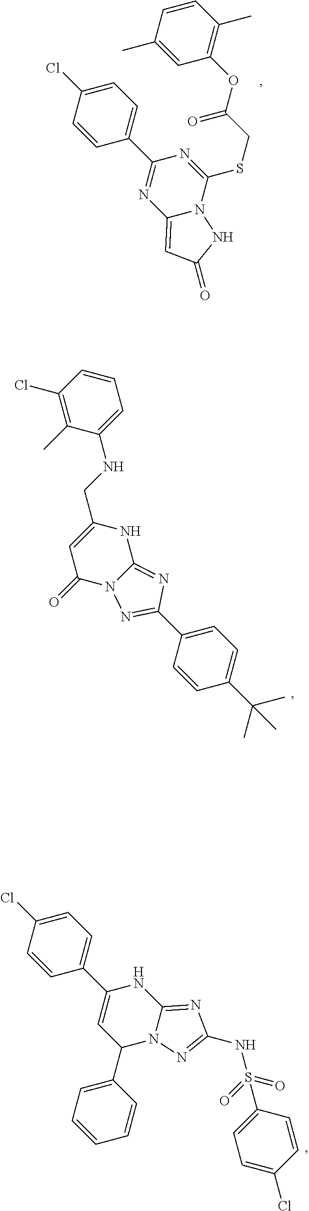 [SMILES: Cc1ccc(C)c(OC(=O)CSc2nc(-c3ccc(Cl)cc3)nc3cc(=O)[nH]n23)c1 ; Cc1c(Cl)cccc1NCc1cc(=O)n2nc(-c3ccc(C(C)(C)C)cc3)nc2[nH]1 ; O=S(=O)(Nc1nc2n(n1)C(c1ccccc1)C=C(c1ccc(Cl)cc1)N2)c1ccc(Cl)cc1]

or a pharmaceutically acceptable salt thereof.

In some cases, the human exonuclease 1 inhibitor compound is:

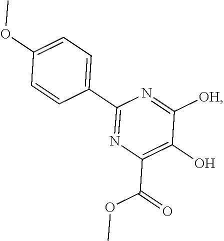 [SMILES: COC(=O)c1nc(-c2ccc(OC)cc2)nc(O)c1O]

or a pharmaceutically acceptable salt thereof.

In some cases, the human exonuclease 1 inhibitor compound is:

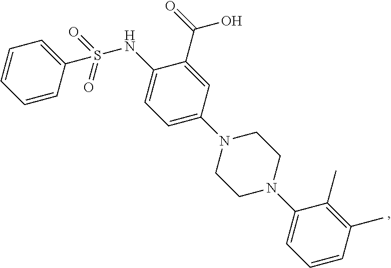 [SMILES: Cc1cccc(N2CCN(c3ccc(NS(=O)(=O)c4ccccc4)c(C(=O)O)c3)CC2)c1C]

or a pharmaceutically acceptable salt thereof.

### Pharmaceutical Compositions

In some aspects, the present disclosure relates to a pharmaceutical composition comprising the human exonuclease 1 inhibitor compound as described herein, or pharmaceutically acceptable salts thereof, and pharmaceutically acceptable excipient. For example, the excipient may be a binder, a filler, a disintegrating agent, a lubricant, a glidant, a plasticizer, a sweetening agent, a flavoring agent, or a combination thereof.

Binders suitable for use in the pharmaceutical compositions provided herein include, but are not limited to, sucrose, starches such as corn starch, potato starch, or starches such as starch paste, pregelatinized starch, and starch 1500, PEG 6000, methocel, WALOCEL®walocel HM, Luvitec®, Luvi-caparolactam, AVICLE®vicel, Succinimidyl-4-(N-maleimidomethyl cyclohexane)-1-carboxylate (SMCC), UNI-PURE®, gelatin, natural and synthetic gums such as acacia, sodium alginate, alginic acid, other alginates, tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, polyvinyl pyrrolidone, hydroxy-propyl methyl cellulose, (e.g., Nos. 2208, 2906, 2910), microcrystalline cellulose, and mixtures thereof. Suitable forms of microcrystalline cellulose include, but are not limited to, the materials sold as AVICEL® PH 101, AVICEL® PH 103 AVICEL® RC 581, AVICEL® PH 105 (available from FMC Corporation, American Viscose Division, Avicel Sales, Marcus Hook, Pa.), and mixtures thereof. In some cases, the binder may be a mixture of microcrystalline cellulose and sodium carboxymethyl cellulose. Suitable anhydrous or low moisture excipients or additviesadditives include AVICEL® PH 103 and Starch 1500 LM.

Examples of fillers suitable for use in the pharmaceutical compositions provided herein include, but are not limited to, talc, calcium carbonate (e.g., granules or powder), sugars such as dextrose, sucrose, lactose, a salt such as calcium carbonate, calcium phosphate, sodium carbonate, sodium phosphate, starches, microcrystalline cellulose, powdered cellulose, cellulosic bases such as methyl cellulose, carboxymethyl cellulose dextrates, kaolin, mannitol, silicic acid, sorbitol, starch, pregelatinized starch, and mixtures thereof.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. In some embodiments, the disintegrant may be deep in the oral solid dosage form to delay disintegration. The amount of disintegrant used varies based upon the type of formulation, and is may be readily discernible to those of ordinary skill in the art. Disintegrants that can be used in the pharmaceutical compositions provided herein include, but are not limited to, agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, magnesium stearate or potassium stearate, ethyl oleate, ethyl laureate, agar, and mixtures thereof. Additional lubricants include, for example, a syloid silica gel (AEROSIL® 200, manufactured by W.R. Grace Co. of Baltimore, Md.), a coagulated aerosol of synthetic silica (marketed by Degussa Co. of Plano, Tex.), CAB-O-SIL® (a pyrogenic silicon dioxide product sold by Cabot Co. of Boston, Mass.), Q7-9120 (Dow Corning), and mixtures thereof.

Plasticizers may be added to control the softness or pliability of oral dosage forms such as shell of a capsule, caplet or a tablet and thus, may improve the mechanical properties of the pH-sensitive materials of the coatings on the oral dosage forms. Suitable plasticizers, include, without limitation, petroleum oils (for e.g., a paraffinic process oil, a naphthenic process oil, and an aromatic process oil), squalene, squalane, plant oils, (e.g., olive oil, camelia oil, castor oil, tall oil, and a peanut oil), silicon oils, dibasic acid esters, (e.g., dibutyl phthalate, and dioctyl phthalate), liquid rubbers (e.g., polybutene and a liquid isoprene rubber), liquid fatty acid esters (e.g., isopropyl myristate ISM), hexyl laurate, diethyl sebacate, and diisopropyl sebacate, triethyl citrate, triacetin, diethylene glycol, polyethylene glycols, polypropylene glycol, phthalates, sorbitol, glycol salicylate, crotaminton, and glycerin or mixtures thereof. The amount of plasticizer may vary depending upon the chemical composition of the pharmaceutical preparation. In one embodiment, the at least one plasticizer is sorbitol, dimethyl isosorbide, or a glycerol.

Examples of glidants include, but are not limited to, colloidal silicone dioxide, cellulose, calcium phosphate, di or tri-basic and the like.

Acceptable sweeteners or sweetening agents include sucrose, saccharin, dextrose, maltose, sugar substitutes, aspartame, xylitol, mannitol, cyclamate, sucralose, maltitol, sorbitol, acesulfame K and the like.

Examples of flavoring agents include peppermint, methyl salicylate, peppermint, spearmint, methyl salicylate, raspberry, red berry, strawberry, pineapple, orange, cherry, and the like.

In some cases, wherein the pharmaceutical composition may be formulated for oral, parenteral, topical, and intraductal delivery. In some cases, the composition may be formulated for oral delivery and may be a tablet, a caplet, a capsule, a pill, a powder, a troche, an elixir, a suspension, a syrup, a wafer, a chewing gum, a dragee, or a lozenge.

### Methods of Treating

In some aspects, the present disclosure relates to a method of treating cancer in a subject, comprising administering to the subject the human exonuclease 1 inhibitor compound as described herein. In some aspects, the present disclosure relates to a method of treating cancer in a subject, comprising administering to the subject a pharmaceutical composition comprising the human exonuclease 1 inhibitor compound as described herein.

In some cases, the human exonuclease 1 inhibitor compound or pharmaceutical composition may be administered to a subject in need thereof once a day, twice a day, thrice a day, four times a day, every other day, twice a week, weekly, fortnightly, twice a month, monthly, quarterly, once every six months, or annually.

In some cases, the cancer may be breast cancer. In some cases, the cancer may be ovarian cancer. In some cases, the cancer may be prostate cancer. In some cases, the cancer may be leukemia cancer. In some cases, the cancer may be melanoma cancer. In some cases, the cancer may be pancreatic cancer. In some cases, the cancer may be cancer of the central nervous system. In some cases, the cancer may be lung cancer. In some cases, the cancer may be kidney cancer.

In some cases, the compound has a IC₅₀ of about 1.0 μM to about 65.0 μM against human exonuclease 1 protein. For example, the IC₅₀ may be about 1.0 μM, about 5.0.0 μM, about 15.0 μM, about 30.0 μM, about 50.0 μM, about 65.0 μM, or any number within a range of between two preceding numbers.

In some cases, administration of the compound is intracutaneous, subcutaneous, intravenous, intraperitoneal, intraarterial, intrathecal, intracapsular, intraorbital, intracardiac, intradermal, transdermal, transtracheal, subcuticular, intraarticular, subcapsular, subarachnoidal, intraspinal, intrasternal, oral, sublingual, buccal, rectal, vaginal, nasal or ocular, or by infusion, inhalation, or nebulization.

In some cases, a therapeutically effective amount of the inhibitor compound is administered to the subject. For example, a therapeutically effect amount, is any amount of the inhibitor compound that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent promotes cancer regression in a subject. In preferred embodiments, a therapeutically effective amount of the drug promotes cancer regression to the point of eliminating the cancer. Promoting cancer regression means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of the drug preferably inhibits cell growth or tumor growth by at least about 20%, more preferably by at least about 40%, even more preferably by at least about 60%, and still more preferably by at least about 80% relative to untreated subjects. In other preferred embodiments of the invention, tumor regression may be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for “immune-related” response patterns.

Effective amounts of the inhibitor compound described herein depend, for example, on the therapeutic purpose, the route of administration, and the condition of the patient. Therefore, it is preferable for a therapist to titer the dosage and modify the route of administration as necessary to obtain the optimal therapeutic effect. For example, a daily dose may be about 0.0001 mg/kg, 0.001 mg/kg, 0.01 mg/kg, 0.1 mg/kg, 1 mg/kg, 10 mg/kg of the patient's body weight, depending on the factors mentioned above. Up to 100 mg/kg, 1000 mg/kg, 10,000 mg/kg or more. The dose is 0.0001 mg/kg to 20 mg/kg, 0.0001 mg/kg to 10 mg/kg, 0.0001 mg/kg to 5 mg/kg, 0.0001 to 2 mg/kg, 0.0001 to 1 mg/kg, 0.0001 mg/kg to 0.75 mg/kg, 0.0001 mg/kg to 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg, or 0.01 to 0.10 mg/kg obtain. In some cases, the inhibitor compound may be administered until a dosage is reached that achieves the desired effect.

A therapeutically effective amount of a drug includes a prophylactically effective amount, which is any amount of the inhibitor compound that, when administered alone or in combination with another therapeutic agent to a subject at risk of developing a cancer (e.g., a subject having a pre-malignant condition) or of suffering a recurrence of cancer, inhibits the development or recurrence of the cancer. In preferred embodiments, the prophylactically effective amount prevents the development or recurrence of the cancer entirely. Inhibiting the development or recurrence of a cancer means either lessening the likelihood of the cancer's development or recurrence, or preventing the development or recurrence of the cancer entirely.

In some cases, the compound inhibits cancer cell proliferation, cancer cell viability or a combination thereof.

### Kit

In some cases, the present disclosure may be directed to a kit comprising the human exonuclease 1 inhibitor compound, or the pharmaceutical composition, as described herein and instructions for use.

The invention may be further understood by the following non-limiting examples.

### EXAMPLES

The following materials and methods may be used for Examples 1-10.

### Differential Expression Analysis

The mRNA expression profiles and correlated clinical data from 33 types of cancer samples and corresponding normal tissue samples were downloaded from TCGA (https://www.cancer.gov/about-nci/organization/ccg/research/structural-genomics/tcga), which involved 11,315 samples in all. The differentially expressed genes (DEGs) between tumors and normal tissues were identified using log 2 transformation and t-tests in the TCGA cohorts with a p-value <0.05. The correlation coefficient was calculated from the cancer types which displayed significant differential expression. The Pearson correlation coefficient was computed to assess the linear relationship between EXO1 and all other genes in the dataset. The p-value was calculated using a two-tailed t-test under the null hypothesis that the correlation coefficient is zero. From this database, the top 100 genes that were most strongly correlated with EXO1 in cancer were identified. Kyoto Encyclopedia of Genomics (KEGG) analyses were subsequently performed for these 100 genes.

### FRET-Based EXO1 Activity Assay and Dose-Response Analysis

In some aspects, a cell-free in vitro assay to detect EXO1 exonuclease activity based on FRET quenching may be described. Briefly, a nick substrate was labeled with a 6-carboxyfluorescein (FAM) fluorophore at the 5′ end of a downstream oligo. A ZEN® quencher and IowaBlack® FQ quencher (Integrated DNA Technologies (IDT), Coralville, Iowa) were attached to the downstream oligo internally and at the 3′ end, respectively, to suppress FAM signal. Two quenchers were used to provide better quenching effects than a single quencher, as recommended by the manufacturer (IDT). The sequence of the nick substrate was as follows: Up-Primer: GCATCCTAAGCCATGCGTGGC (SEQ ID NO: 2); Template: CCTAAAAGTTTCACTCAGGACCACGCATGGCTTAGGATGC (SEQ ID NO: 3); Probe: /56-FAM/TCCTGAGTG/ZEN/AAACTTTTAGG/3IABkFQ/(SEQ ID NO: 4). When EXO1 cleaves the nick substrate, the FAM-labeled nucleotide may be untethered from the substrate and yields a bright green fluorescence. The EXO1 inhibitory capacity of each compound was identified by performing the assay in a 384-well plate format and incubating the FAM-labeled substrate (200 nM) with reaction buffer only (blank; 50 mM HEPES-KOH (pH 7.5), 45 mM KCl, 5 mM MgCl2, 1 mM DTT, 100 μg/mL BSA) or EXO1 (0.25 nM) in the presence of 1% v/v DMSO (vehicle) or candidate EXO1 inhibitor compound in reaction buffer at 37 °C. The initial screening of each compound was performed at 10 μM and followed by a dose response analysis to ascertain the IC₅₀ for each compound which displayed significant EXO1 inhibition at the initial concentration. Fluorescent signals were measured after 30 min incubation. Samples from the initial FRET-based EXO1 activity assay were collected for polyacrylamide gel analysis to confirm that increases in FAM signal were due to inhibition of EXO1 activity and not extraneous factors, such as inhibitor color interference or quencher technology errors.

Data was normalized by subtracting the relative fluorescence unit (RFU) of the blank sample from that of each EXO1-containing reaction. The relative activity of individual compounds was subsequently calculated by setting the corrected RFU of EXO1 with DMSO as 100% and comparing the corrected RFU for each condition to the DMSO value. Each assay was performed by sequentially adding inhibitor compound, EXO1 enzyme, and DNA substrate to minimize starting differences. Of the disclosed EXO1 inhibitor compounds, hits were chosen if an IC₅₀≤20 μM was observed in the dose-response analysis. Following IC₅₀ screening, an analogous FRET-based assay was performed using purified FEN1 to assess the EXO1 specificity of each compound.

### In Silico Pharmacokinetic Profiling Using SwissADME

The pharmacokinetic and drug-likeness of individual inhibitor candidates was evaluated using the SwissADME web tool (http://www.swissadme.ch). The canonical SMILES representations of each compound were manually generated using the Research Collaboratory for Structural Bioinformatics Protein Data Bank (RCSB PDB) Chemical Sketch Tool (https://www.rcsb.org/chemical-sketch) and used as input for the SwissADME interface. Output data was collected for the following physicochemical properties: lipophilicity (Log P (using X LOG P3)), size (molecular weight (MW)), polarity (topological polar surface area (TPSA)), water solubility (Log S (using ESOL)), saturation (fraction of sp3 carbons (Fsp3)), and flexibility (number of rotatable bonds). Optimal ranges for each of these six parameters were pre-defined according to SwissADME's Bioavailability Radar plot, which was developed based on data adapted from the analysis of orally bioavailable drugs. Accordingly, the optimal ranges for each property were considered as follows: Log P (XLOGP3) between −0.7 and +5.0; MW between 150 and 500 g/mol; TPSA between 20 and 130 Å2; Log S (ESOL) between −6 and 0; Fsp3 between 0.25 and 1; and number of rotatable bonds between 0 and 9.

### Virtual Prediction of EXO1 Inhibitor Binding Sites

Druggable sites were predicted on the X-ray crystal structure of hEXO1 (PDB: 5V08) using an in-house-developed Druggable Site Prediction by FDA-approved drugs (DSP) methodology, as previously described. Briefly, a diverse subset of 100 FDA-approved drug molecules were docked around the protein surface to predict the optimal binding site(s) on the protein surface. Probable inhibitor binding sites were determined based on the locations which contained the highest number of FDA-approved drugs bound to the site.

### Small Molecule Compounds and Human Cell Lines

Each human cancer cell line was cultured according to guidelines from the American Type Culture Collection (ATCC). The corresponding cell culture mediums were supplemented with 10% fetal bovine serum (FBS) (CYTIVA®, Marlborough, Massachusetts) and antibiotic solution (THERMO FISHER SCIENTIFIC®, Waltham, Massachusetts) to a final concentration of 100 U/mL penicillin and 100 μg/mL streptomycin. Cells were continuously incubated at 37 °C with 5% CO₂, except for during passaging, seeding, or physical manipulation of cells for experimental purposes. For high-throughput screening, compounds were acquired from the in-house City of Hope High-Throughput Screening core facility compound library. For subsequent experiments, fresh powders of individual hit compounds were obtained from ChemDiv® and camptothecin (CPT) from Selleck® Chemicals (Houston, Texas).

### Thermal Shift Assay

To assess for changes in the thermal stability of EXO1 protein in response to treatment with candidate small molecule inhibitors or DMSO (vehicle control), the thermal shift assay (Applied Biosystems™ Protein Thermal Shift™ Dye Kit, Cat #4461146) was utilized. The protein melt reaction mix consisted of freshly prepared 10× thermal shift dye, purified EXO1 enzyme (1 μg/uL), thermal shift buffer, DMSO or inhibitors, and sterilized MILLI-Q® water (MERCK©, Rahway, New Jersey). A drug-free supermix was pre-made and dispensed in 96-well polymerase chain reaction (PCR) plates followed by the addition of DMSO or inhibitors with increasing concentration. The reaction plate was heated from 10 °C to 98 °C at a rate of 0.5 degree per 10 second cycle and the fluorescence intensity of individual wells was measured at 490/530 nm using a Bio-Rad® CFX96 Touch™ Real-Time PCR System (Bio-Rad® Laboratories, Inc., Hercules, California). The fluorescence measurements of three technical replicates per treatment condition were then averaged to calculate melting temperature shift (ΔTm) as a readout for compound binding affinity.

### Modeling of Inhibitor Binding Kinetics

To characterize the reaction rate kinetics of compounds F684 and C200 on EXO1 via Michaelis-Menten modeling, a FRET-based EXO1 activity assay at a set inhibitor concentration with varying substrate concentrations was performed. The initial velocity of enzyme activity was obtained by calculating the rate of the linear region of the time dependent relative fluorescence unit (RFU). To observe the change in maximum reaction rate (Vmax) and Michaelis constant (Km), the IC₅₀ was used to determine an inhibitor concentration with approximately 50% inhibition and compared it to no inhibitor (0 μM) to confirm. Subsequently, a Lineweaver-Burk plot was generated to confirm the changes in Vmax and Km.

### Molecular Docking Analysis

Three-dimensional structural modeling was performed to assess the binding position of the disclosed inhibitors to EXO1 using the crystal structure of human wild-type (WT) EXO1 in complex with 5′ recessed-end DNA and Mn2+ (PDB: 3QEB). Molecular docking of each compound was accomplished using the Schrodinger® Maestro™ software (Schrodinger LLC, New York, New York). Briefly, the preferred binding site and individual EXO1 residue interactions for the most potent representative compound from each of the seven inhibitor scaffolds was evaluated by performing rigid (Glide) and induced-fit docking for of each compound at the DSP-derived putative inhibitor binding sites, Site 1 and Site 2. In addition to comparing docking scores to determine their preferred binding site, the hydrogen bonds, salt bridges, and pi stacking interactions were catalogued for each compound to determine which residues may be most critical for inhibitory activity. The residues with the highest number of direct interactions with the inhibitors and/or frequently located within 5 Å of the inhibitors at Site 1 and Site 2 were selected as candidates for mutagenesis studies.

The docking scores of each of the seven scaffold representatives were further evaluated against X-ray crystallography-resolved structures of the EXO1 protein at nine distinct stages of its exonucleolytic reaction cycle to determine if the EXO1 inhibitor candidates preferentially bind to a specific conformation of EXO1. For this analysis, PDB structures: 5UZV, 5V04, 5V05, 5V06, 5V07, 5V08, 5V09, 5VOA, and 5VOB were used. Docking scores for each compound at Site 1 and Site 2 were compared, and the preferential binding conformations were defined as those with the most negative docking scores.

### Protein Expression and Purification

The N-terminal domain of human EXO1 (hEXO1) (residues 1-345; SEQ ID NO: 1) was cloned into the pET28b vector. The following mutations were introduced via site-directed mutagenesis based on the findings of molecular docking analysis: K85A, E89A, R92A, I125A, D225A, and K292A K294A following the established protocol (oligonucleotide primers listed in Table 1).

TABLE 1
Oligonucleotide Primers

| SEQ | | |
|---|---|---|
| ID | | |
| NO. | Description | Sequence |
| 5 | Lys85Ala Forward | TCTTCTAGATCTCTCTACTTCCTTTGCAGAAGGTAA |
| | Primer | AGTACATCCATCAAAT |
| 6 | Lys85Ala Reverse | ATTTGATGGATGTACTTTACCTTCTGCAAAGGAAGT |
| | Primer | AGAGAGATCTAGAAGA |
| 7 | Glu89Ala Forward | CGTCTTTCTCTTCTAGATCTCGCTACTTCCTTTTTAG |
| | Primer | AAGGTA |
| 8 | Glu89Ala Reverse | TACCTTCTAAAAAGGAAGTAGCGAGATCTAGAAGA |
| | Primer | GAAAGACG |
| 9 | Arg92Ala Forward | GATTGGCTTGTCGTCTTTCTCTTGCAGATCTCTCTAC |
| | Primer | TTCCTTTTTAG |
| 10 | Arg92Ala Reverse | CTAAAAAGGAAGTAGAGAGATCTGCAAGAGAAAG |
| | Primer | ACGACAAGCCAATC |
| 11 | Ile125Ala Forward | GGGCCATGGCATGTGTGGCATTGATAGACCGGGTG |
| | Primer | AA |
| 12 | Ile125Ala Reverse | TTCACCCGGTCTATCAATGCCACACATGCCATGGCCC |
| | Primer | |
| 13 | Asp225Ala Forward | CCCGGTCTATCAATGCCACACATGCCATGG |
| | Primer | |
| 14 | Asp225Ala Reverse | CCATGGCATGTGTGGCATTGATAGACCGGG |
| | Primer | |
| 15 | Lys292Ala/Lys294Ala | CTTCATAGGCGTTCAGAGGAATAAGTGCCCTTGCGA |
| | Forward Primer | TGGGATCAAAAACTAGCTGATAGA |
| 16 | Lys292Ala/Lys294Ala | TCTATCAGCTAGTTTTTGATCCCATCGCAAGGGCAC |
| | Reverse Primer | TTATTCCTCTGAACGCCTATGAAG |

Wild-type and mutant hEXO1 N-terminal domains were expressed in E. coli BL21(DE3) and Rosetta 2(DE3). The transformed bacteria were then transferred to lysogeny broth (LB) (BD Difco™, Becton, Dickinson and Company, Franklin Lakes, New Jersey) containing kanamycin (100 μg/mL) and shaken continuously (220 rpm) at 37 °C overnight. For large scale expression, the overnight bacterial growth was adjusted to 0.1 optical density at the 600 nm wavelength (OD600) using kanamycin-containing LB broth and incubated, with continuous shaking, at 37 °C until the culture reached an optical density (OD) between 0.6 and 0.8. EXO1 expression was then induced by adding 0.4 mM isopropyl β-d-1-thiogalactopyranoside (IPTG) and placed in a shaking incubator for 16 h at 16 °C. EXO1-expressing bacteria were then harvested via centrifugation (8,000×g, 4 °C, 15 min), resuspended in 10 mL of lysis buffer containing 20 mM HEPES-NaOH, pH 7.5; 100 mM NaCl; 20 mM imidazole, pH 8; 5 mM 2-mercaptoethanol (BME); 10% glycerol; 1 mM PMSF; and 1× protease cocktail. The bacterial cells were subsequently lysed by sonication for 10 sec (×30 cycles at 50% amplification), the resulting lysate was centrifuged (15,000×g) at 4 °C for 15 min and the clear supernatant was isolated for purification. The recombinant EXO1 protein was purified using an ÅKTA Pure™ chromatography system (CYTIVA®, Marlborough, Massachusetts). The protein sample was loaded into a 1 mL HisTrap™ Ni-Sepharose column (CYTIVA®, Marlborough, Massachusetts) and equilibrated with a binding buffer containing 20 mM HEPES-NaOH, pH 7.5; 100 mM NaCl; 20 mM imidazole, pH 8; 5 mM BME; and 10% glycerol. The protein-bound column was then washed with 5 column volumes of the binding buffer. A linear gradient of an elution buffer consisting of 20 mM HEPES-NaOH, pH 7.5; 100 mM NaCl; 500 mM imidazole, pH 8; 5 mM BME; and 10% glycerol was used to strip the bound recombinant protein from the column. The eluted fractions containing hEXO1 were identified using sodium dodecyl sulfate-polyacrylamide gel electrophoresis (SDS-PAGE) and pooled. To ensure the purity of the protein was suitable for downstream assays, the pooled EXO1-containing sample was diluted with heparin loading buffer (20 mM HEPES-NaOH, pH 7.5; 100 mM NaCl; 1 mM EDTA; 5 mM BME; 5% glycerol), loaded onto a 1 mL heparin column (HiTrap® Heparin HP, GE Healthcare Life Sciences, Marlborough, Massachusetts), and a linear gradient of heparin elution buffer (20 mM HEPES-NaOH, pH 7.5; 1 M NaCl; 1 mM EDTA; 5 mM BME; 5% glycerol) was used to strip the bound recombinant protein from the column. The fractions containing pure hEXO1 were identified using SDS-PAGE and those with ≥90% purity were pooled. The pooled sample was concentrated in a 10,000 MWC Centricon® concentrator (MILLIPORE SIGMA©, Rahway, New Jersey) to a final volume of 0.5 mL. The resulting protein concentrate was aliquoted, flash frozen in dry ice, and immediately stored at −80 °C for future use. The wild-type human FEN1 enzyme was purified as previously described.

High-throughput drug screening, QC evaluation, and data analysis For high-throughput screening via the FRET-based exonuclease activity assay, 40 nL of compounds from the ChemDiv® (Selleck® Chemicals (Houston, Texas)) collection, dissolved in sterile DMSO, were added into empty 384-well black-wall clear-bottom plates using an acoustic dispenser Echo™ (Beckman Coulter® Inc., Brea, California), followed by adding purified EXO1 protein at 2.2 ng in 30 μL volume per well. After incubation at room temperature for 20 min, 10 μL of the 6-carboxyfluorescein (FAM) fluorophore-labeled DNA substrate was added to the reaction into each well, resulting in the final concentration of substrate at 125 nM and the final compound concentration at 10 μM. Plates were incubated at 37 °C for 30 min and the fluorescent signal was measured at 485 nm/528 nm using a Ensight™ multi-mode microplate reader (PerkinElmer®, Waltham, Massachusetts). A final concentration of 10 μM DMSO (0.1% v/v) was included in each screening plate as a negative control. To analyze the high throughput screening data, the raw fluorescence readouts, along with plate ID, drug concentration, and plate format, were uploaded to CBIS (Cheminnovation.com) for analysis. Quality control (QC) assessment was conducted, which involved calculating coefficient of variation (CV) of DMSO controls, signal-to-background (S/B) ratio and assay Z′ factor. CV of 3.81% for DMSO, S/B of 8.8 and Z′ factor of 0.84 were achieved, which are all well within the industrial standard of <10% of CV, S/B>4 and Z′ factor>0.5. The percentage of activity for each compound was determined by comparing it to the mean readout of DMSO controls of each plate. Hit selection thresholds were set at >50% inhibition compared to DMSO control, and 84 preliminary hits were selected.

### Hit Confirmation, Validation, and Counter Screening

For subsequent hit confirmation, 84 compounds from the primary screen were selected and subjected to a dose-dependent response assay, starting at 100 μM with three-fold serial dilution. To analyze the data from the dose-dependent response assay, IC₅₀ values for each hit were calculated through nonlinear regression analysis using GraphPad Prism® 10 (GraphPad Software, Inc., San Diego, California). Of the initial 84 compounds, 20 compounds demonstrated IC₅₀ values less than 20 μM and were isolated for further validation. Subsequently, a counter screen was performed to eliminate potential false positive hits. To accomplish this, an analogous FRET assay was established using purified FEN1 enzyme in place of EXO1. To be considered a hit compound, IC₅₀ values in the EXO1 assay were required to be less than 20 μM and the IC₅₀ ratio between FEN1 and EXO1 must be more than five-fold.

### Immunofluorescence Staining

Cells were seeded at a density of 3×10⁵ cells per well in 12-well plates with pre-placed coverslips and treated with or without various inhibitor treatments overnight. Cells were then incubated with 5-ethynyl-2′-deoxyuridine (EdU) for 30 minutes before collection. For PARylation analysis, cells were permeabilized with 0.1% Triton X100, fixed with 4% paraformaldehyde, blocked with the Image-iT™ FX signal enhancer (Invitrogen®, Waltham, Massachusetts), and incubated (1.5 h, room temperature) with the indicated primary antibodies (PAR/pADPr Antibody; Bio-Techne®, Minneapolis, Minnesota). The cells were then washed with phosphate buffered saline (PBS) buffer and incubated (1 h, room temperature) with the corresponding secondary antibodies (1:200, Invitrogen®, Waltham, Massachusetts). Subsequently, the fixed cells were incubated with Click-iT™ reaction mix (100 mM Tris-HCl pH 8.5, 10 mM azide-Alexa 488, 1 mM CuSO4 and 100 mM ascorbic acid (Sigma Aldrich®, Saint Louis, Missouri) for 1 h at room temperature. The slides were then washed with PBS buffer, counterstained with 4′,6-diamidino-2-phenylindole (DAPI) and analyzed with a fluorescence microscope (Zeiss® Axio™ Observer Z1, Zeiss Group, Oberkochen, Germany). For γ-H2AX staining, cells were fixed with 4% paraformaldehyde, blocked with the Image-iT™ FX signal enhancer (Invitrogen®, Waltham, Massachusetts), and incubated (1.5 h, room temperature) with the indicated primary antibodies (γ-H2AX). The cells were then washed with PBS buffer and incubated (1 h, room temperature) with the corresponding secondary antibodies (1:200, Invitrogen®, Waltham, Massachusetts). The slides were then washed with PBS buffer, Click-iT™ reaction mix for EdU as aforementioned, counterstained with DAPI, and analyzed with a fluorescence microscope (Zeiss® Axio™ Observer Z1, Zeiss Group, Oberkochen, Germany).

### Gel-Based Phospho-RPA End Resection Assay

To examine the impact of EXO1 inhibition on DSB end resection, MDA-MB-231 cells were treated with 0 M, 20 μM, or 40 M of compound C200 or F684 overnight and subsequently exposed to no camptothecin (CPT) or 2 M CPT for 2 h to cause replication fork stalling and induce DNA single-strand breaks (SSBs) which progress to DSBs in S-phase. The treated cells were then lysed in SDS-PAGE sample buffer and the lysate was separated by SDS-PAGE prior to immobilization on a nitrocellulose membrane and western blotting. Antibodies against unphosphorylated RPA, S33-P-RPA2, and tubulin were subsequently assayed according to the manufacturer's recommendations. The ratio of P-RPA:RPA levels were quantified via ImageJ.

### Homologous Recombination Assay

To evaluate the effects of C200 and F684 on HR, U2OS cells stably expressing an HR-GFP reporter were utilized as previously described. Cells were infected with adenovirus expressing I-SceI restriction enzyme for 16 h to induce DNA double strand breaks (DSBs). Following viral infection, the culture medium was replaced with fresh medium containing small molecule inhibitors. Cells were treated with 0, 10, 50, or 100 M of C200 or F684, either alone or in combination with the DNA2 inhibitor C5 (10 μM) and incubated for an additional 48 h. Subsequently, cells were harvested, and green fluorescent protein (GFP)-positive frequencies, indicative of homologous recombination (HR) repair events, were quantified using a CyAn™ ADP Analyzer (Beckman Coulter® Inc., Brea, California) Experiments were performed in triplicate, and data are presented as mean±standard deviation (s.d.).

### National Cancer Institute Human Tumor Cell Line Drug Profiling

The cytotoxicity of representative compounds from the seven EXO1 inhibitor scaffolds were tested against a subset of the NCI-60 cell line panel along with additional three pancreatic cell lines (FG, AsPC1, CaPan2). In general, cells were seeded in 384 well tissue culture-treated plates overnight and treated with various concentrations of each hit compound (a serial three-fold dilution from 50 μM for all tested hits). Each concentration was performed in duplicate. Cell viability was then measured by CellTiter-Glo® (Promega Corp., Madison, Wisconsin) after 72 h of treatment. The IC₅₀, defined as the drug concentration that decreased viability by 50%, was calculated using nonlinear regression analysis via GraphPad Prism® 10 (GraphPad Software, Inc., San Diego, California).

### Annexin V Apoptosis Assay

MDA-MB-436 parental cells (BRCA1−), and MDA-MB-436 expressing WT BRCA1 (BRCA1+), were seeded into 384-well clear bottom black plates (Catalogue No. 781090, Greiner Bio-One, USA) at a density of 1,500 cells per well in 30 μL of RPMI-1640 medium followed by incubating at 37 °C 5% CO₂. After 24 h of incubation, testing compounds were added to cells at final concentrations of 50 M, 25 M, 12 M, and 5 M using the Echo™ Liquid Handler (Beckman Coulter® Inc., Brea, California). Subsequently, 10 μL of NIR-labelled Annexin V apoptosis reagent (Cat. #4768, Sartorius Group, Germany) was added to each well at a 1:200 final dilution. The plates were then transferred to The Incucyte® Live-Cell Analysis System (Sartorius Group, Germany) for real-time apoptosis monitoring over a five-day period. Imaging was conducted using a 10× objective with phase-contrast and near-infrared (NIR) fluorescent channels, capturing scans every 2 h. Image analysis was performed with Incucyte® software, and the total NIR fluorescence intensity was quantified to evaluate apoptosis.

### In Vitro Cell Survival Assay

The survival rate of MDA-MB-436 parental cells (BRCA1−) and WT BRCA1 expressing MDA-MB-436 (BRCA1+) cells were assessed by cell counting. For this assay, 10,000 cells were seeded per well in a 24-well plate and incubated in complete culture medium containing DMSO or the EXO1 inhibitor of interest at 37 °C with % CO₂. The medium was refreshed every three to four days by replacing it with fresh medium containing DMSO or EXO1 inhibitor. After five days, the cells were trypsinized, and the survival curve was generated by manual cell counting.

### In Vivo Tumor Growth Assay

The tumor growth rate of human breast cancer cells with or without treatment with EXO1 inhibitor was evaluated using a xenograft breast cancer mouse model. Six- to eight-week-old male or female NSG (NOD SCID gamma) mice were grafted with 4,000,000 MDA-MB-436 (BRCA1−) or MDA-MB-436 (BRCA1+) cells per mouse. EXO1 inhibitor F684 was started once the tumor volume reached 100 mm³. Vehicle control or F684 (8.5 mg/kg body weight) were administered daily via intraperitoneal injection. The tumor volume in each mouse was measured twice a week and calculated using the formula of (length×width×width)/2 (mm³). When the tumors reached a volume of 1,000 mm³, the mice were euthanized. Animal studies were conducted in accordance with an approved protocol adhering to the IACUC policies and procedures at the City of Hope.

### Statistical Analysis

All experimental values are represented as the average±standard deviation of at least three technical replicates for three independent experiments unless indicated otherwise. In cell-based assays, statistical significance for each experimental condition was assessed by comparison to DMSO-treated (vehicle control) samples via Student's t-test. Sample groups with p<0.05 were deemed significantly different from DMSO controls.

### Example 1: Analysis of EXO1 Gene Expression in Multiple Cancers

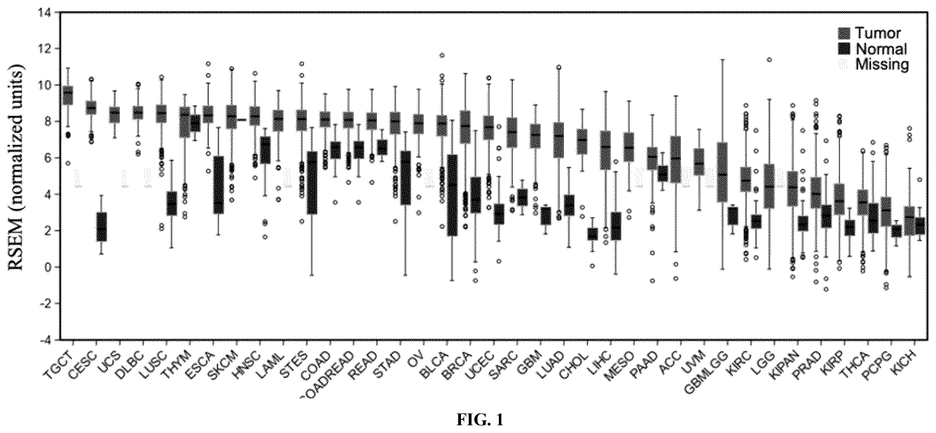

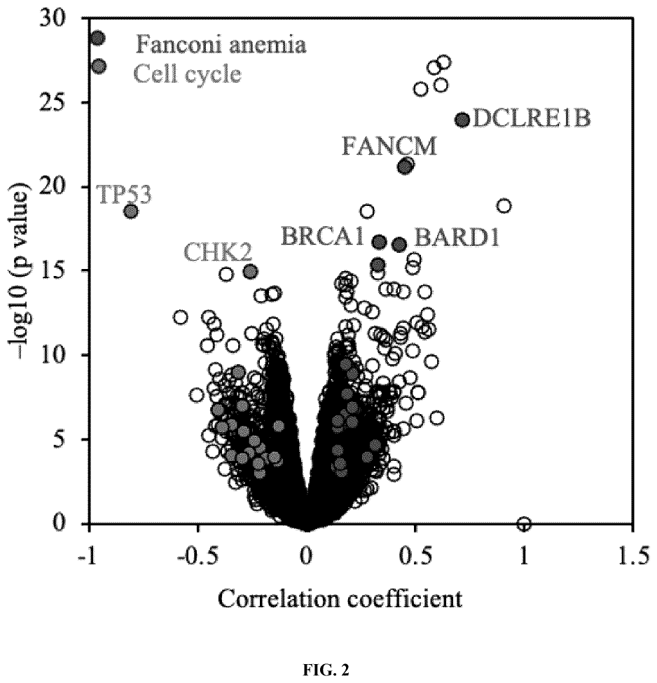

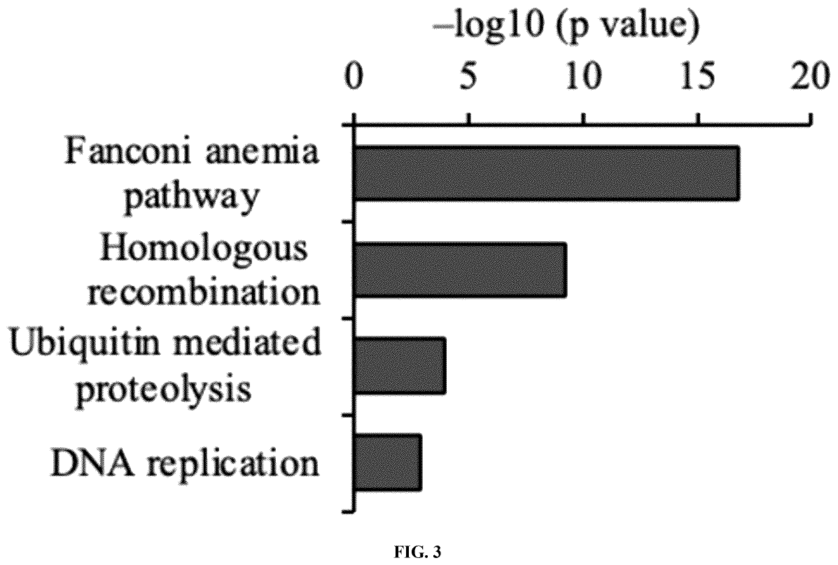

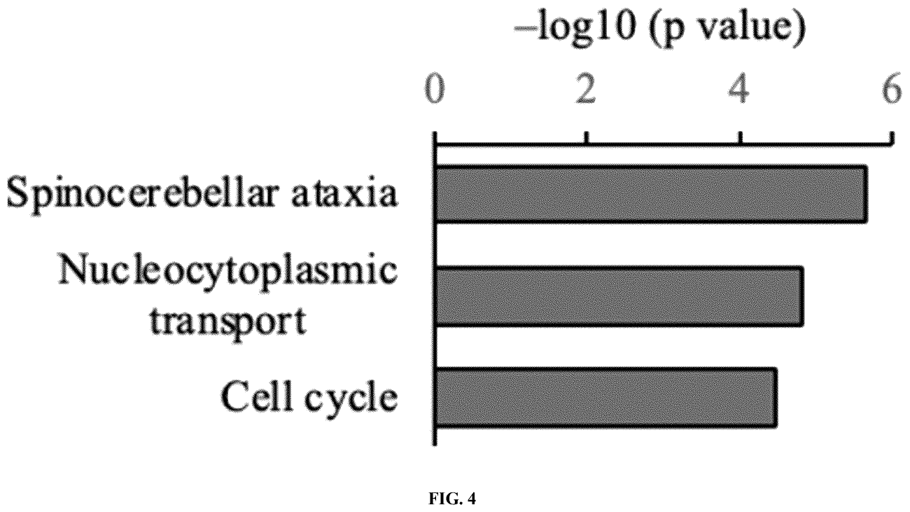

To determine if EXO1 is overexpressed in human cancers, the TCGA database was surveyed for EXO1 expression levels. FIG. 1, shows a comparison of EXO1 mRNA expression profiles in cancer and corresponding normal human tissues from the TCGA database. The top bars represent EXO1 expression in cancer tissues; the bottom bars represent EXO1 expression in corresponding normal human tissues. EXO1 gene expression was found to be elevated in multiple types of cancer, including hormone-related breast, ovarian, and prostate cancers. The correlation coefficient was then calculated to assess the linear relationship between EXO1 and all other genes in the dataset. FIG. 2 shows a volcano map that depicts the differential correlation coefficients for Fanconi anemia (FA) pathway (left side: DCLREIB, FANCM, BRACA1, and BARD1) and cell cycle-related genes (right side: TP53 and CHK2). The FA pathway may be a replication-coupled DNA repair mechanism known to enlist several key proteins involved in homologous recombination. Given that under normal physiology EXO1 activity is restricted to DNA replication and is known to functionally intersect with FA and cell cycle genes, it was unsurprising to see that these pathways were the most highly correlated with EXO1 expression. Kyoto Encyclopedia of Genes and Genomes (KEGG) pathway enrichment analysis also confirmed a strong co-dependency between EXO1 and Fanconi anemia-related genes (FIG. 3) and a negative correlation with cell cycle-related genes (FIG. 4). These findings are consistent with an important role of EXO1 in DNA replication and DNA damage repair, which are aberrantly high in cancer cells relative to their normal tissue counterparts and suggest that EXO1 may be a promising novel target for anti-cancer treatment.

### Example 2: High-Throughput Screening

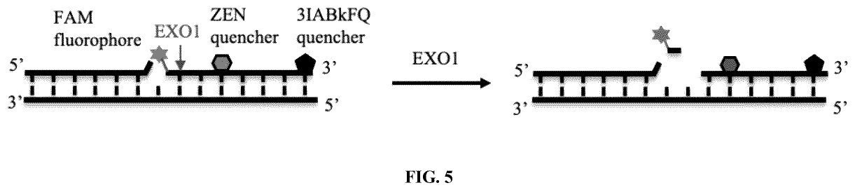

Conventionally, the 32P-labeled substrate-based in vitro nuclease assay is the gold-standard to characterize the nuclease activities of EXO1 and other structure-specific nucleases, including FEN1 and DNA2. Although this conventional assay provides high resolution of DNA cleavage by nucleases, it is unsuitable for high-throughput screening (HTS) for inhibitors due to its low throughput and use of radioisotopes. To overcome these limitations, an in vitro assay for EXO1 exonuclease activity based on fluorescence resonance energy transfer (FRET) quenching, which is amenable to HTS for inhibitors was developed and is described herein. (FIG. 5).

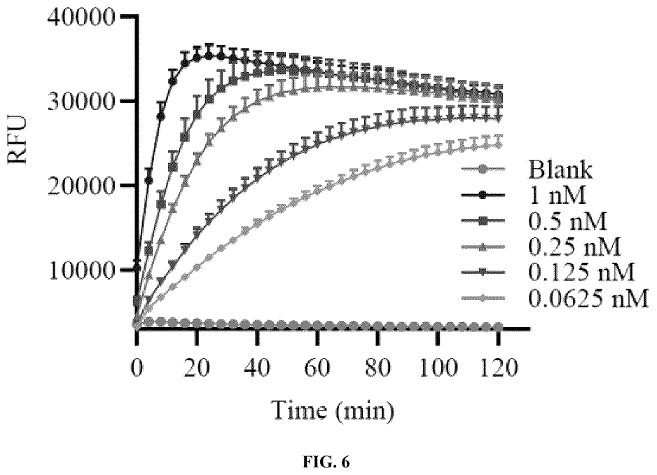

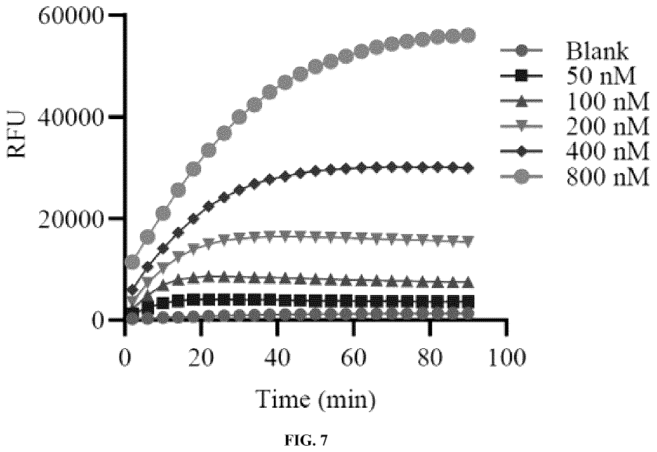

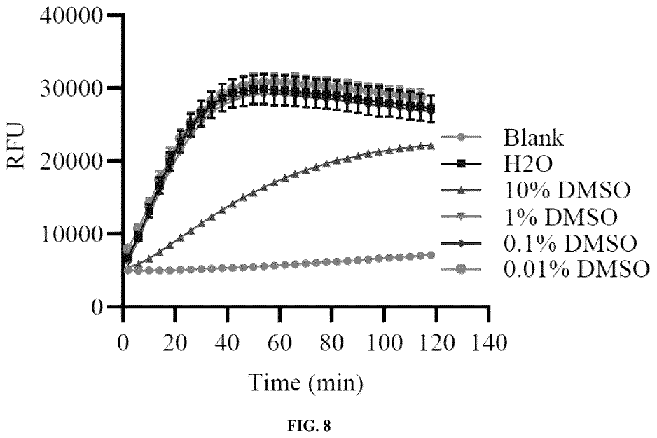

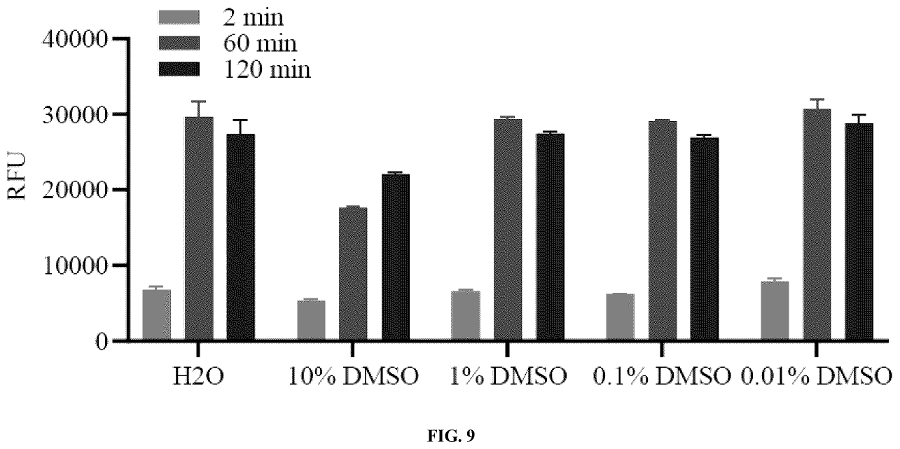

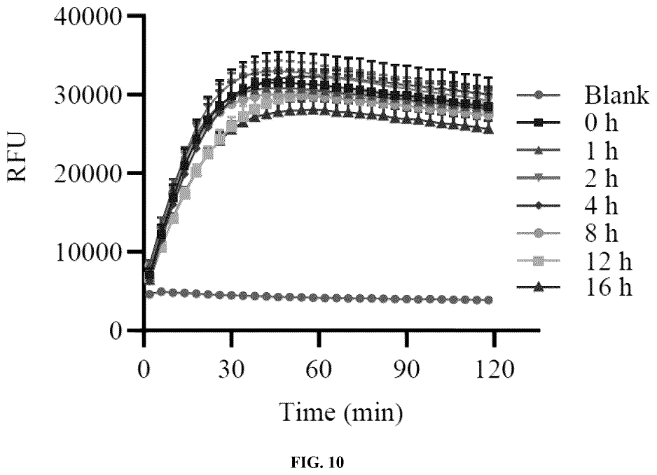

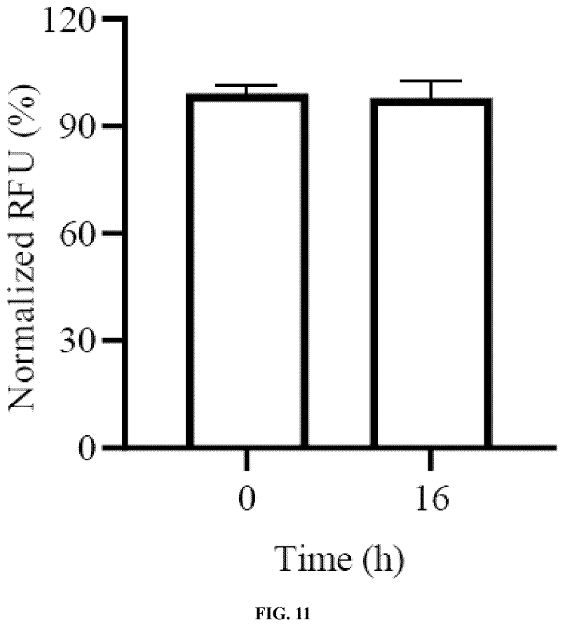

To establish optimal HTS parameters, the kinetics of EXO1 exonuclease activity using the novel FRET-based assay were systematically characterized. Briefly, the nick substrate was labeled with FAM fluorophore at the 5′ end of the downstream oligo, which also has an internal ZEN® quencher and a 3′ IowaBlack® FQ 3IABkFQ quencher (Integrated DNA Technologies (IDT), Coralville, Iowa). Cleavage of the nick substrate by EXO1 lights up FAM to give green fluorescence. It was discovered that at a concentration of 100 μM EXO1, reactions had a sufficiently high ratio (~10-fold) of product to background fluorescence, allowing for detection of as little as 10% product, which is ideal for kinetic analysis. Using 100 nM DNA substrate, the linear regions of the progress curves (relative fluorescence unit [RFU] vs. time) were 10, 15, 20, 30, and 40 min with values being a mean±standard deviation of three independent assays (FIG. 6). Furthermore, the enzymatic activity of EXO1 (100 μM) in the presence of varying concentrations of DNA substrate was evaluated and found that at a 200 nM concentration of the substrate, the fluorescent signal intensity was sufficiently above background with values being a mean±standard deviation of three independent assays (FIG. 7). It was further investigated whether or not DMSO interfered with fluorescence intensity and observed DMSO had minimal impact on EXO1 activity even at final concentration of 10% (v/v) within 120 min (FIG. 8 and FIG. 9). Finally, the stability of purified EXO1 protein in reaction buffer was investigated by maintaining on ice for 1, 2, 4, 8, 12, or 16 h. The retained activity levels were approximately 100%, 100%, 100%, 98%, 85%, and 83% of the activity measured at 0 h, respectively (FIG. 10). The EXO1 protein in stock buffer maintained at room temperature for 16 h still demonstrated full activity as assayed using the described FRET-based assay comparing 16 h versus 0 h. (FIG. 11). Thus 100 μM EXO1 and 200 nM DNA substrate concentrations and up to 30 min. were selected as the reaction endpoint during HTS, so that the reaction remained in the linear phase.

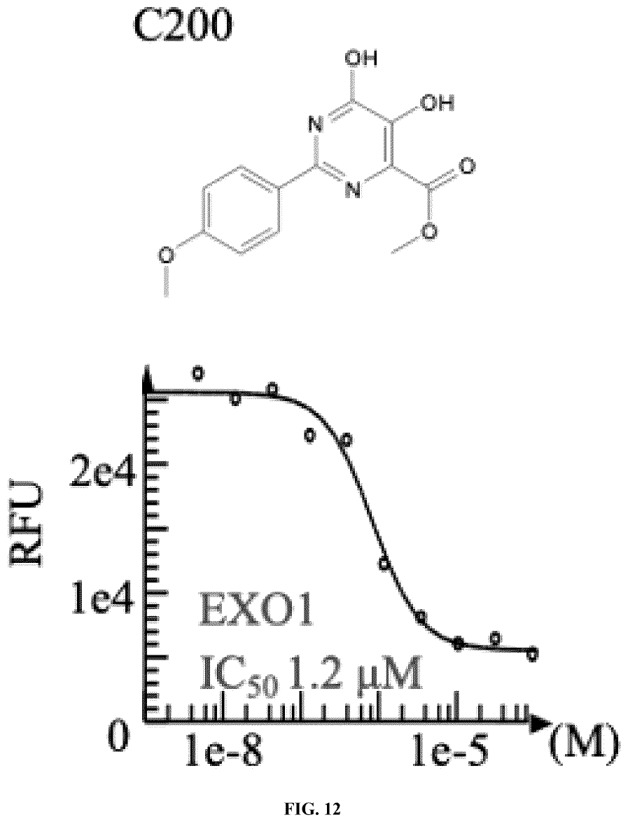

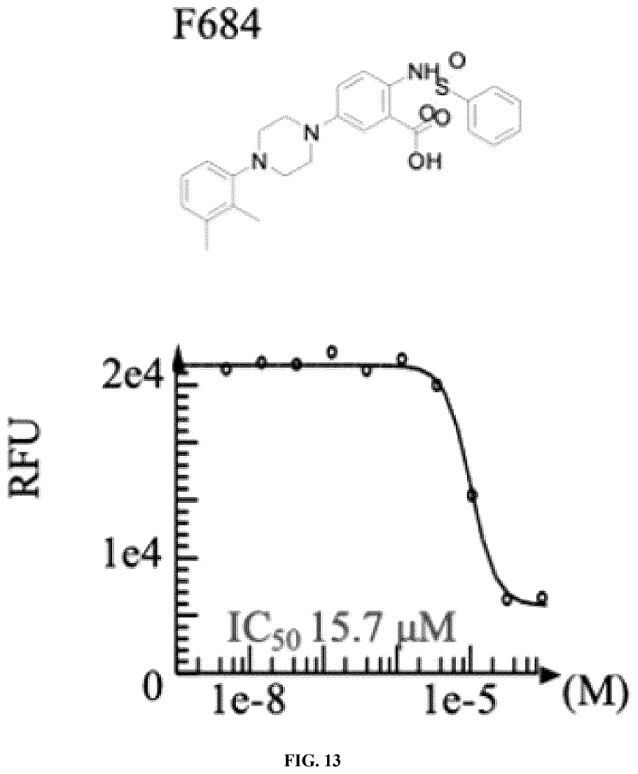

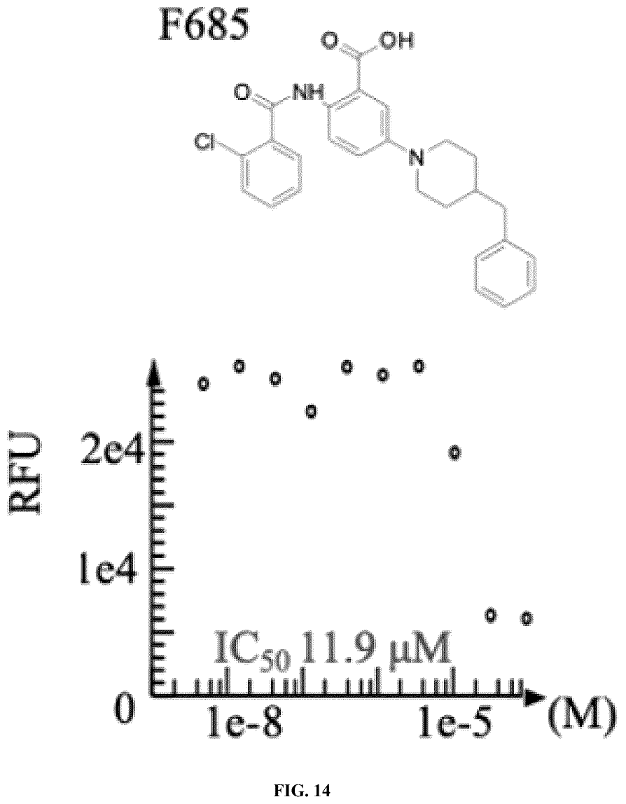

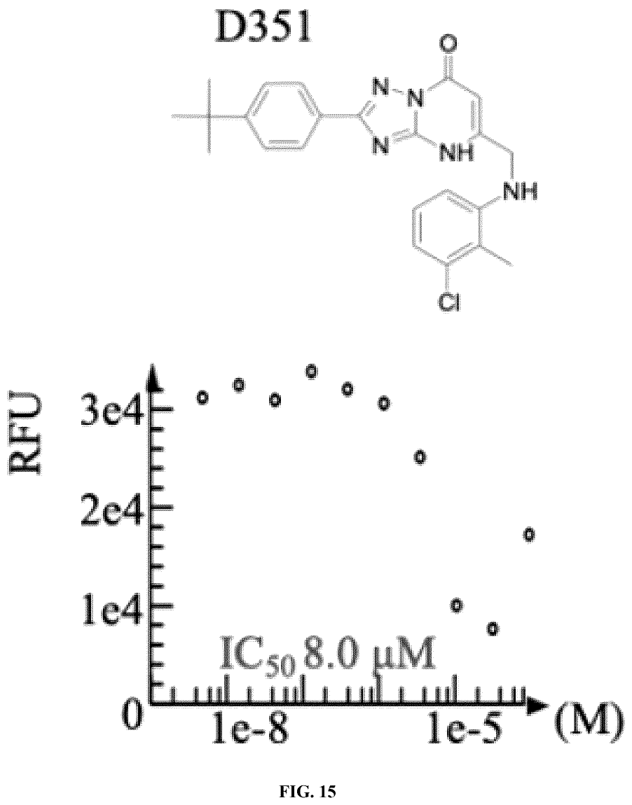

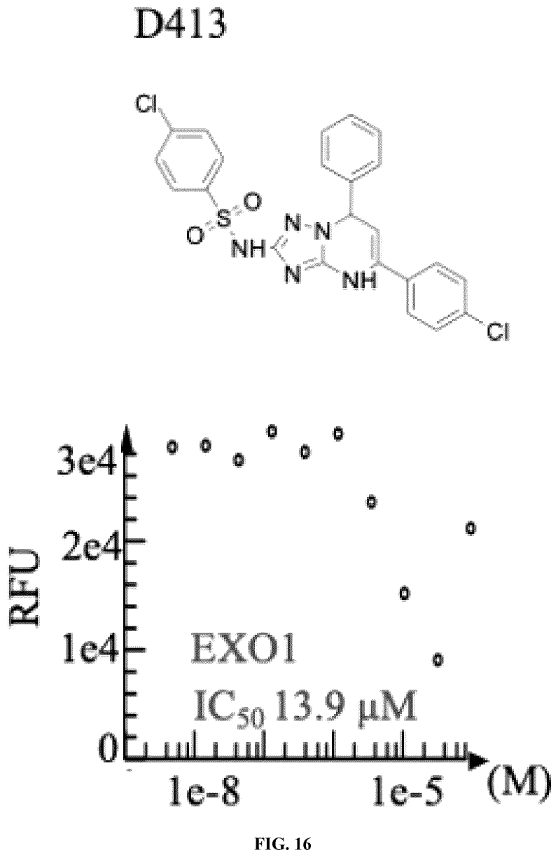

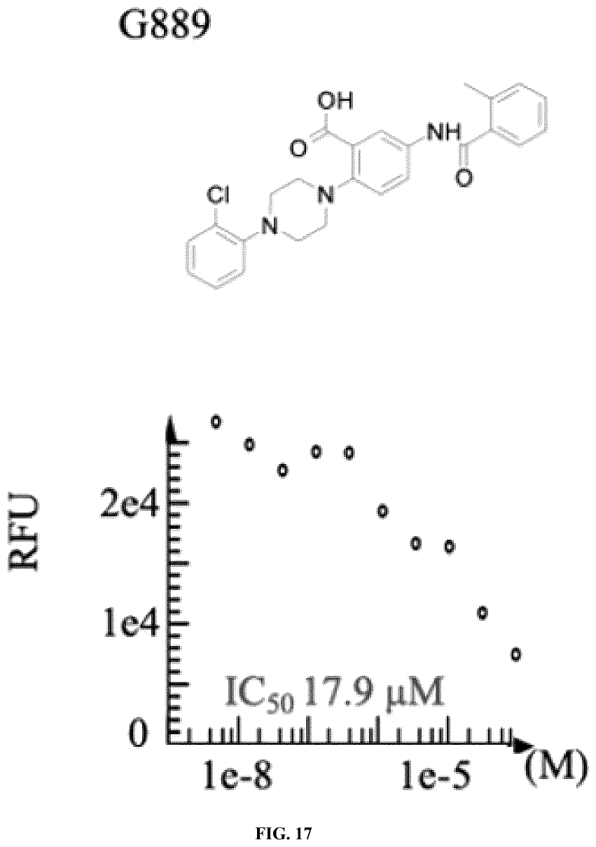

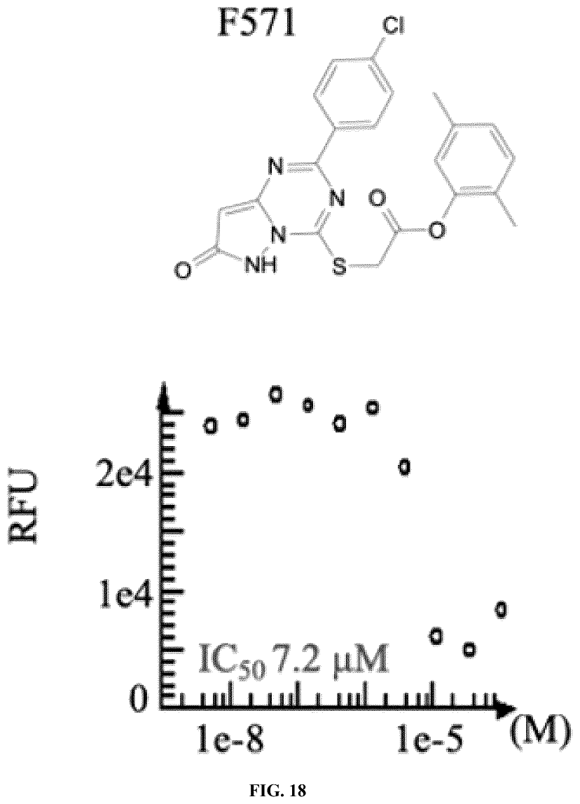

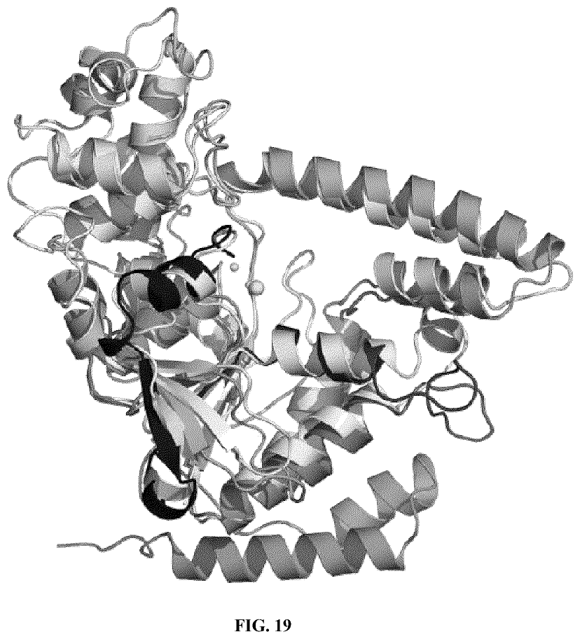

Using the described FRET-based exonuclease assay, a series of EXO1 inhibitors were identified from HTS of 45,000-compound Targeted Diversity library from ChemDiv® (Selleck® Chemicals (Houston, Texas)). There were a total 20 confirmed hits whose inhibitory activity (IC₅₀) against EXO1 was 20 μM or less. These hits were cheminformatically grouped into seven different chemical scaffolds. The best compound within each chemical scaffold is presented as an example in FIGS. 12-18. The specificity of these compounds were further tested by comparing their activities against EXO1 vs. FEN1 protein (FIG. 19), which is functionally related to and occasionally compensated for during replication by EXO1. In yeast, the nuclease domain of EXO1 and FEN1 (Rad27) share 28% identity, while in humans they share 29.8% identity.

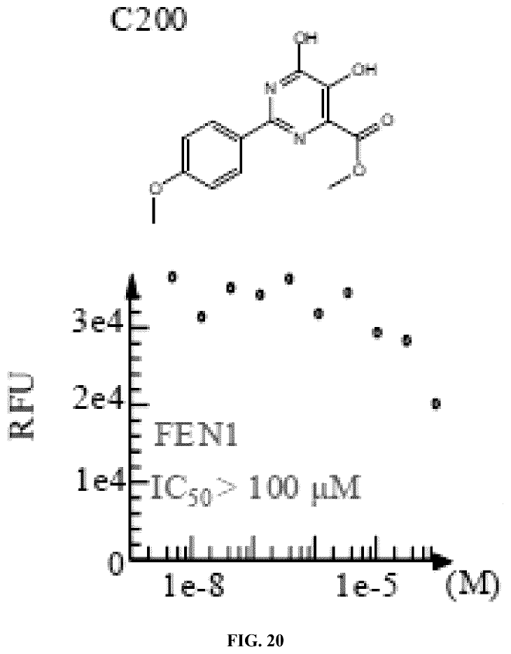

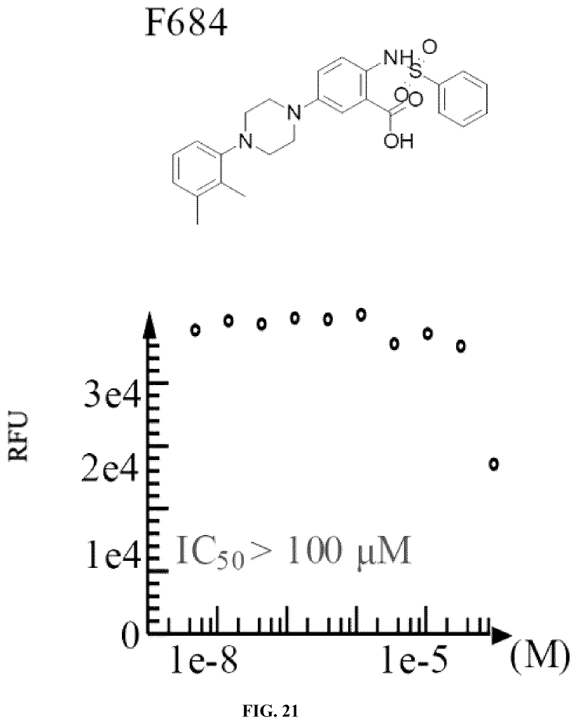

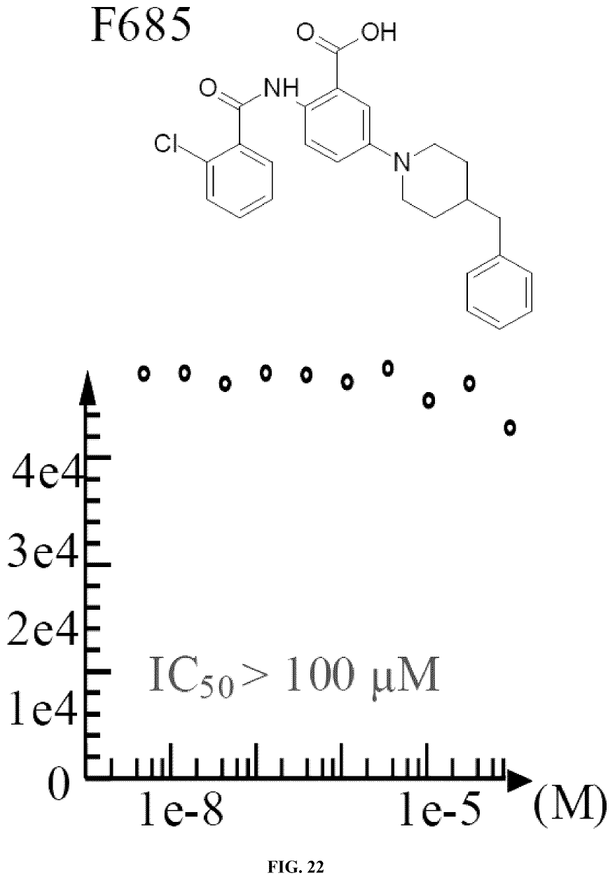

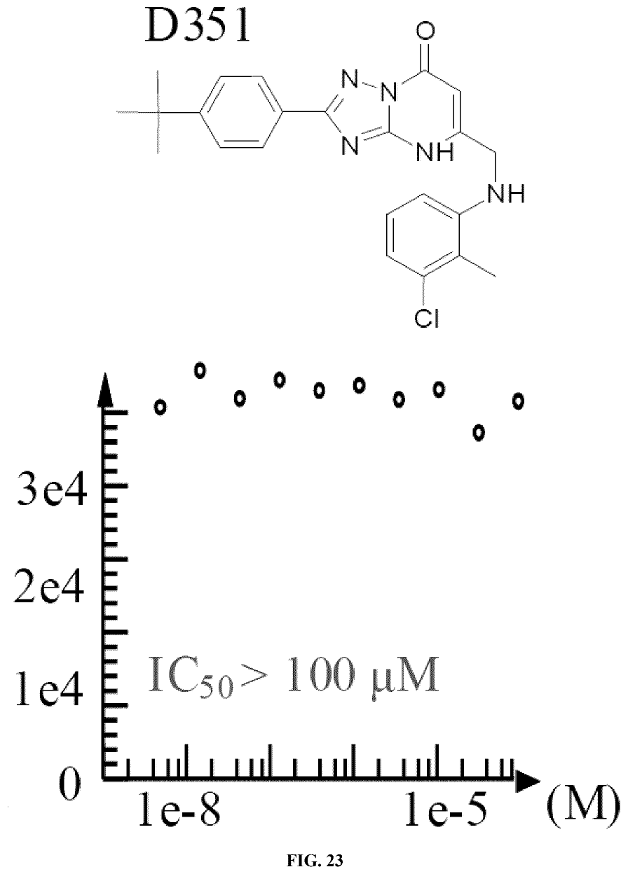

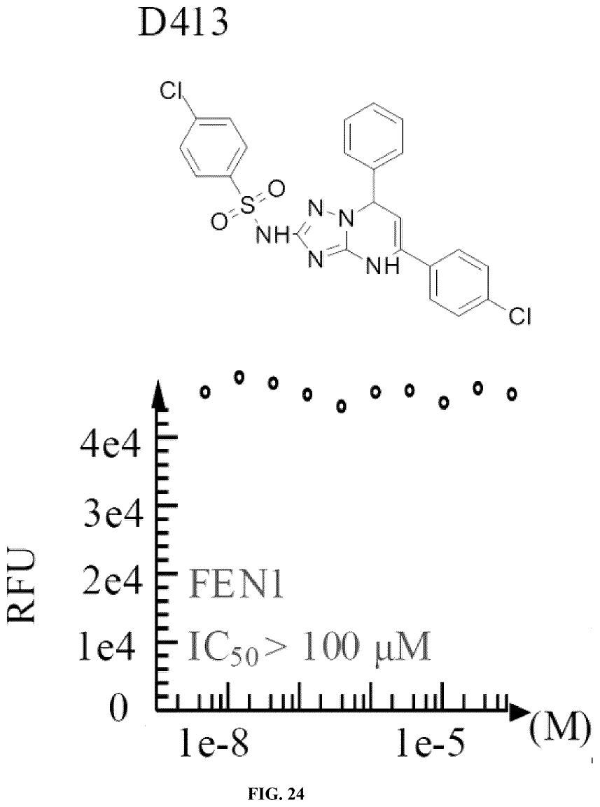

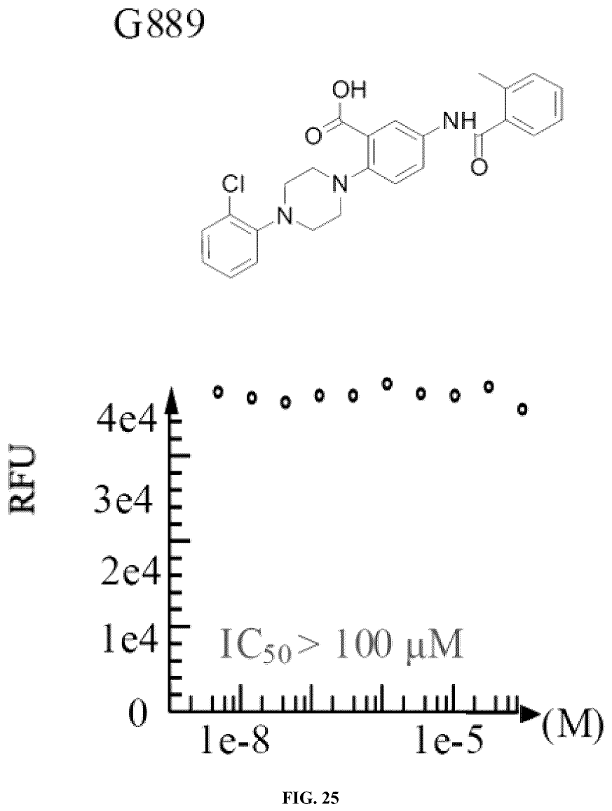

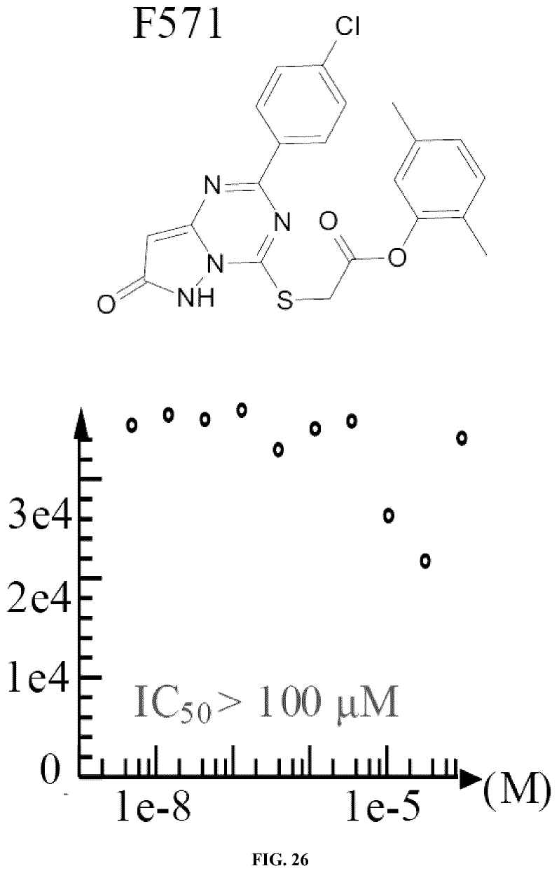

Most of the EXO1 inhibitor scaffolds showed no or weak inhibitory effect on FEN1 exonuclease activity (>100 μM; Table 2 and FIGS. 20-26). Compounds displaying a favored inhibition against EXO1 rather than FEN1 (with the IC₅₀ ratio>5) were prioritized. Most confirmed hits passed this criterion, with the exception of two compounds within the F685 scaffold (FIG. 22). A total of 18 inhibitors showed strong inhibition of EXO1 at low micromolar concentrations, with preferred inhibition against EXO1 rather than FEN1, which may be suggestive of a potential for further structural optimization.

TABLE 2
Selective inhibition and thermal stabilization
of EXO1 by inhibitor candidates.

| Compound | EXO1 IC₅₀ (μM) | FEN1 IC₅₀ (μM) | ΔTₘ (°C) |
|---|---|---|---|
| C200 | 1.2 | >100.0 | 1.3 |
| F684 | 15.7 | >100.0 | 2.5 |
| F685 | 11.9 | >100.0 | 3.0 |
| D351 | 8.0 | >100.0 | 3.0 |
| D413 | 13.9 | >100.0 | 3.5 |
| G889 | 17.9 | >100.0 | 1.5 |
| F571 | 7.2 | >100.0 | 5.0 |

### Example 3: Analysis of Binding Stability

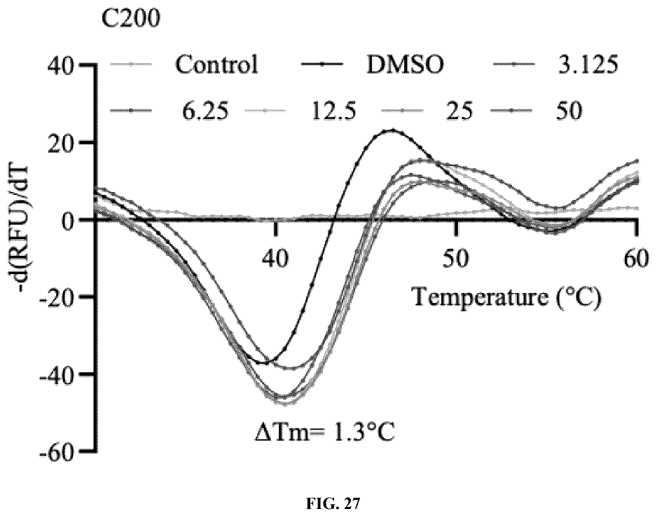

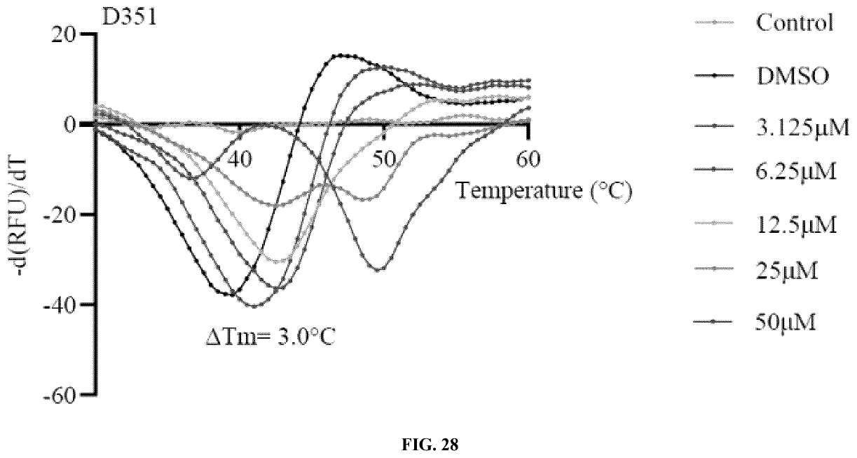

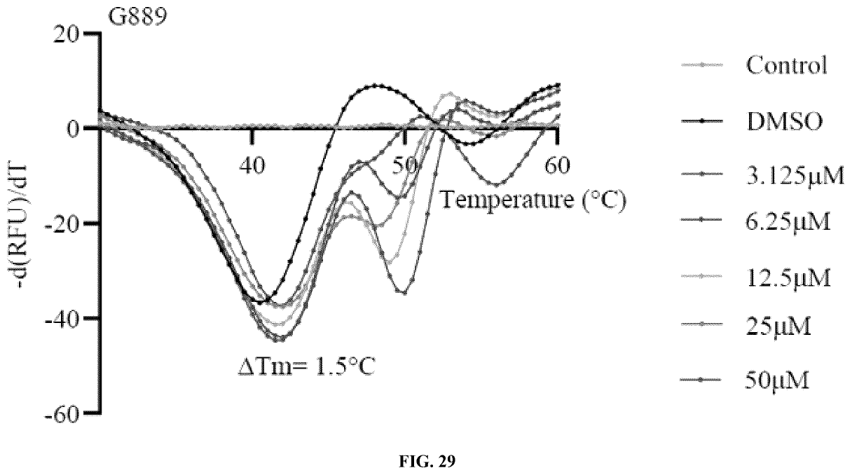

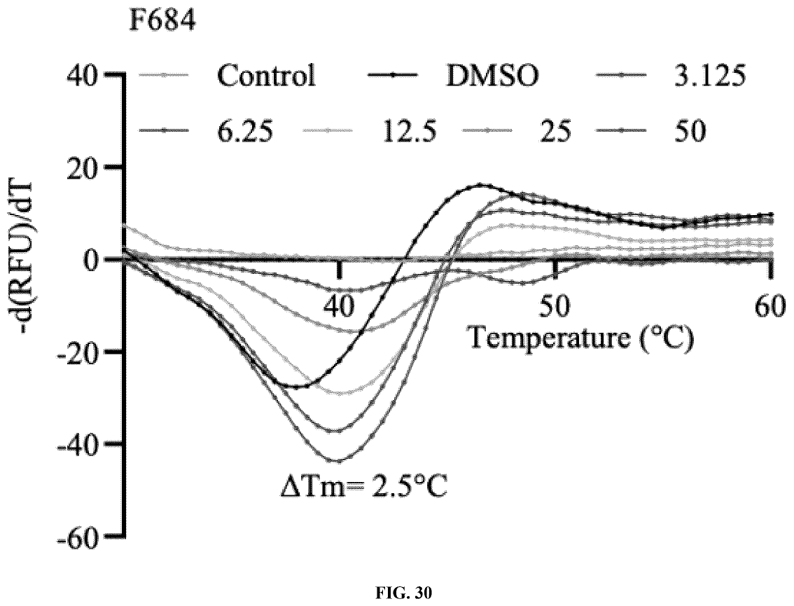

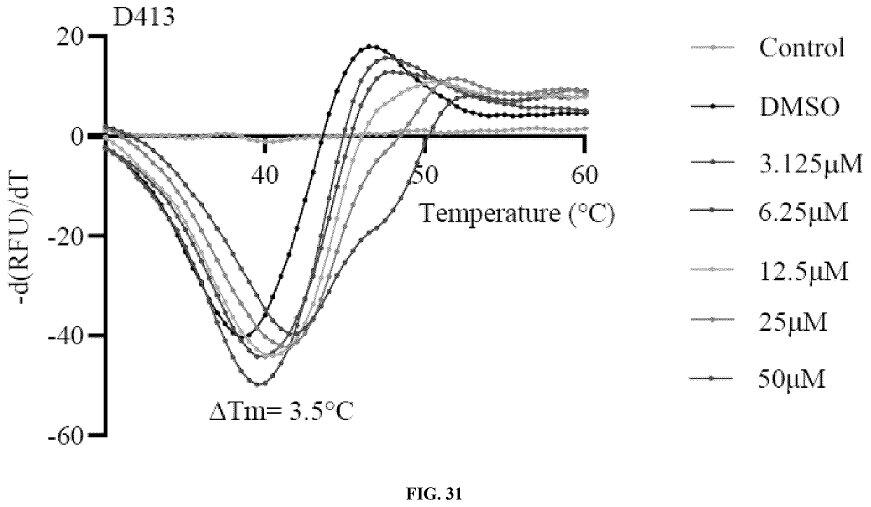

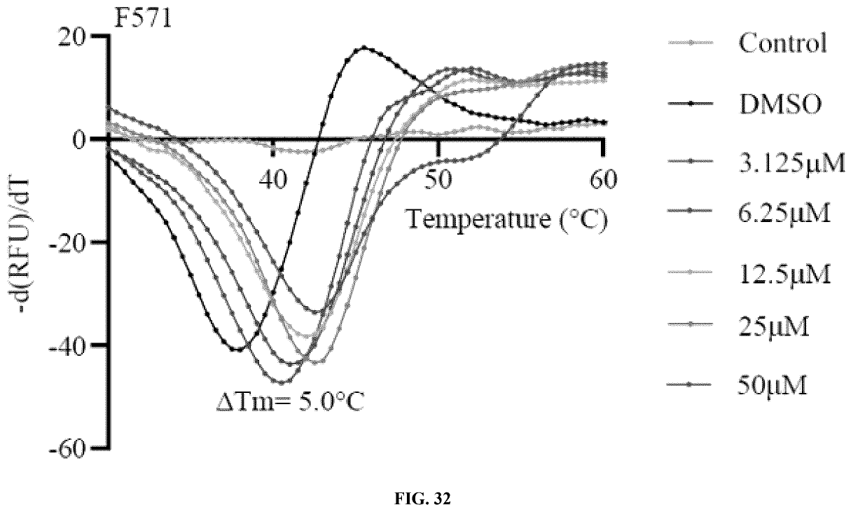

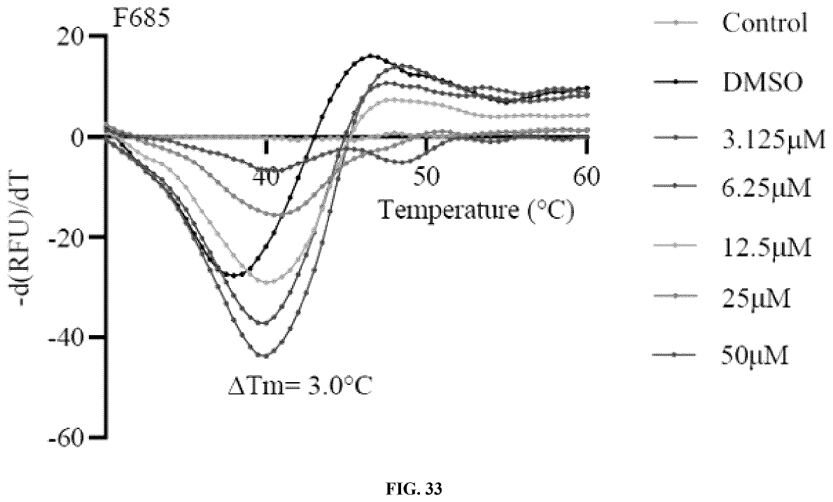

The melt curves and melting temperature (Tm) of the protein in the presence of increasing concentrations of inhibitors were investigated. In each experiment, baseline protein melting temperature was evaluated using an untreated control containing only protein and dye, and thermal shifts were measured by comparing the change in Tm between vehicle (DMSO) and treatment with inhibitor compounds C200 and F684 are depicted in FIG. 27 and FIG. 30 respectively. The curves illustrate the derivative of the thermal shift assay melt curves and display the maximum shift in Tm for each compound. Inhibitors C200 (FIG. 27), D351 (FIG. 28), and G889 (FIG. 29) exhibited a minimal change (~1 °C) in Tm across the tested concentrations, up to 100 μM, suggesting they may bind weakly to EXO1. The molecular weight of compound C200 may classify it as a “fragment” hit (<300 Da). Due to the smaller size and generally weaker binding affinity, fragments typically exhibit lower Tm shifts compared to larger molecules. In contrast, the binding curves for inhibitors F684 (FIG. 30), D351 (FIG. 28), D413 (FIG. 31), F571 (FIG. 32), and F685 (FIG. 33) demonstrate a clear ≥2 °C increase in Tm with higher inhibitor concentrations, indicating a significant stabilizing effect which may be indicative of their binding to the EXO1 protein. Overall, these results provide valuable insights into the strength of binding for each inhibitor at the EXO1 nuclease domain, which could be critical for further drug development and optimization.

### Example 4: Prioritization of EXO1 Inhibitor Compounds

The physicochemical properties of each inhibitor candidate was investigated via the SwissADME web tool to estimate pharmacokinetics and drug-likeness. For comparison, previously disclosed EXO1 inhibitor C73 was also included in the analysis. Six key parameters were considered: lipophilicity (LIPO), size (MW), polarity (POLAR), water solubility (INSOLU), saturation (INSATU), and flexibility (FLEX). Compounds which fall within the optimal ranges for each of these six parameters have been correlated with favorable absorption, distribution, metabolism, and excretion (ADME) profiles. The optimal ranges for each property were considered as follows: LIPO (using XLOGP3) between −0.7 and +5.0; MW between 150 and 500 g/mol; POLAR (TPSA) between 20 and 130 Å2; INSOLU (using Log S (ESOL)) between −6 and 0; INSATU (Fsp3) between 0.25 and 1; and FLEX (number of rotatable bonds) between 0 and 9.

As detailed in Table 3, Swiss ADME analysis revealed that the majority of compounds fell within the optimal range for five out of the six parameters: lipophilicity, size, polarity, water solubility, and flexibility. The only consistently out-of-range property was saturation, which fell below the recommended threshold of 0.25 for all but one compound.

TABLE 3
Physicochemical properties of novel
EXO1 inhibitor candidates and C73

| Compound | LIPO | MW | POLAR | INSOLU | INSATU | FLEX |
|---|---|---|---|---|---|---|
| C200 | 3.21 | 274.27 | 75.99 | −3.74 | −3.74 | 4 |
| F684 | 4.97 | 465.56 | 98.33 | −5.87 | −5.87 | 6 |
| F685 | 6.29 | 448.94 | 69.64 | −6.54 | −6.54 | 7 |
| D351 | 6.31 | 436.96 | 77.88 | −6.48 | −6.48 | 5 |
| D413 | 4.99 | 513.42 | 94.54 | −6.23 | −6.23 | 5 |
| G889 | 4.99 | 449.93 | 72.88 | −5.79 | −5.79 | 6 |
| F571 | 3.66 | 455.94 | 117.45 | −4.86 | −4.86 | 6 |
| C73 | 4.14 | 546.6 | 164.26 | −5.45 | −5.45 | 11 |

Compound C200 had the most promising overall profile, displaying the lowest molecular weight (274.27 g/mol), moderate lipophilicity (XLOGP3=3.21), moderate polarity (TPSA=75.99 Å2), high water solubility (ESOL=−3.74), and moderate flexibility (rotatable bonds=4). Similarly, F684 fell within the acceptable range for size (MW=465.56 g/mol), lipophilicity (XLOGP3=4.97), polarity (TPSA=98.33 Å2), water solubility (ESOL=−5.87), and flexibility (rotatable bonds=6), and outperformed all but one compound (i.e., D351) in terms of saturation (Fsp3=0.24). In contrast, the remaining five scaffolds displayed multiple red flags: F685 and D351 are excessively lipophilic, D413 and C73 have high molecular weights, and F571 and C73 are highly polar. As expected, C73 did not perform well in this analysis, and fell within the optimal range for just two of the six properties (i.e., lipophilicity and water solubility). Accordingly, C200 and F684 were prioritized for further analysis based on their balanced physicochemical profiles and alignment with drug-likeness criteria.

### Example 5: Analysis of Inhibitors EXO1 Activity Via Uncompetitive Mechanism

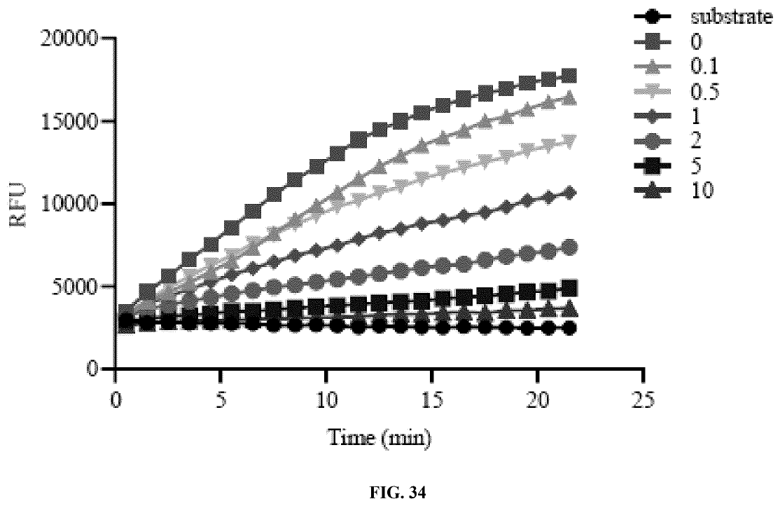

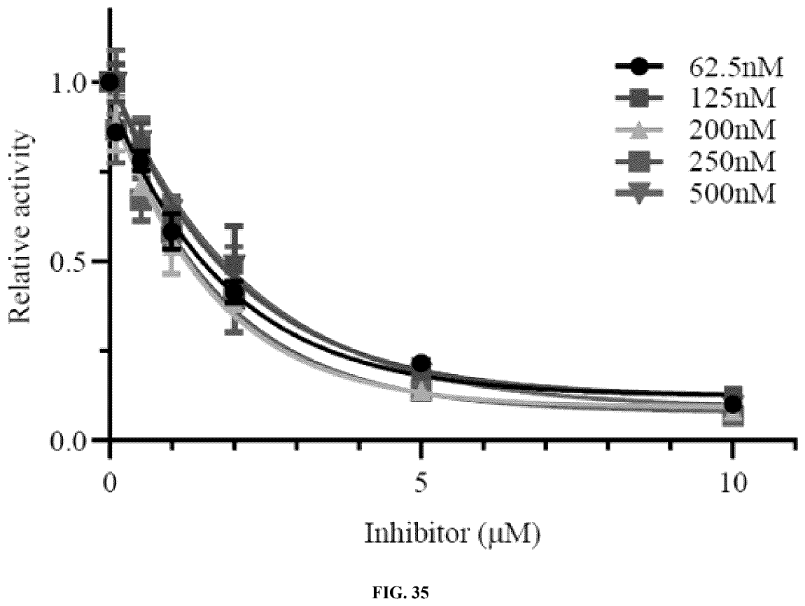

A inhibitor kinetics assay was performed to determine the IC₅₀ of the disclosed EXO1 inhibitors using a flap DNA substrate that was intended for the study of the inhibitor compounds binding mechanism. First, the time course of the nuclease activity was investigated by comparing the reaction rate with and without inhibitor compound 684 treatment to determine the 50% inhibitory concentration for kinetic analysis at a 100 nM DNA substrate concentration (FIG. 34). Next, to evaluate the optimal DNA substrate concentrations for enzyme kinetic assays, the relative activity of EXO1 across varying concentrations of substrate and inhibitor compound 684 was measured and found that the activity was highly consistent across substrate concentrations ranging from 62.5 nM to 500 nM using inhibitor concentrations up to 10 μM (FIG. 35).

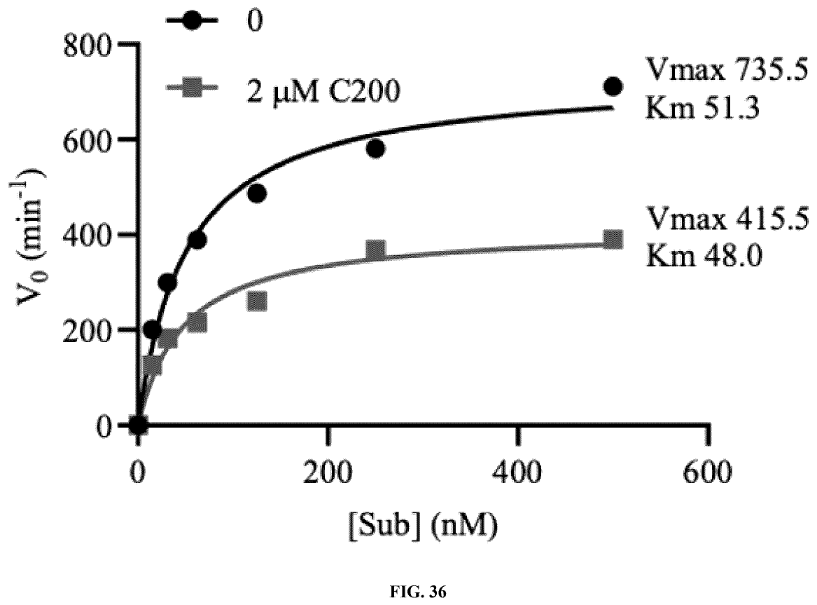

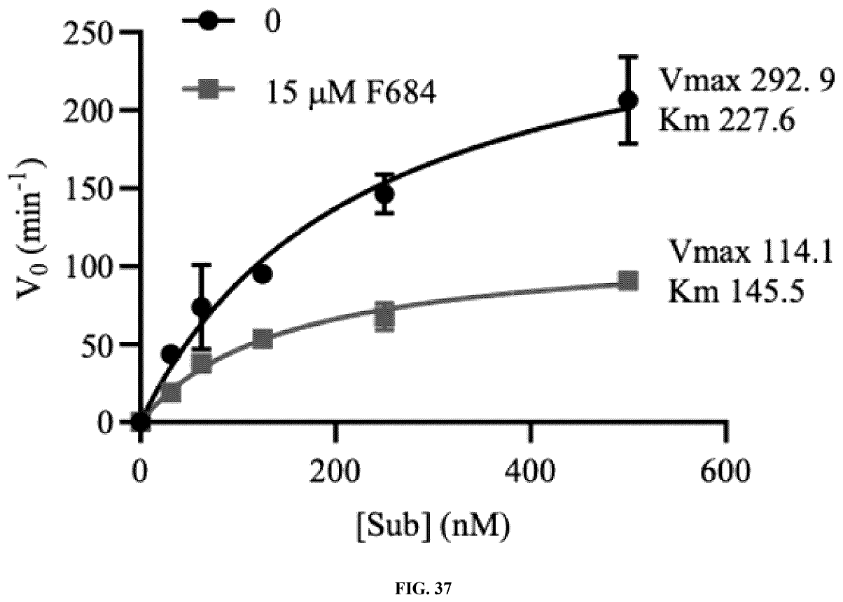

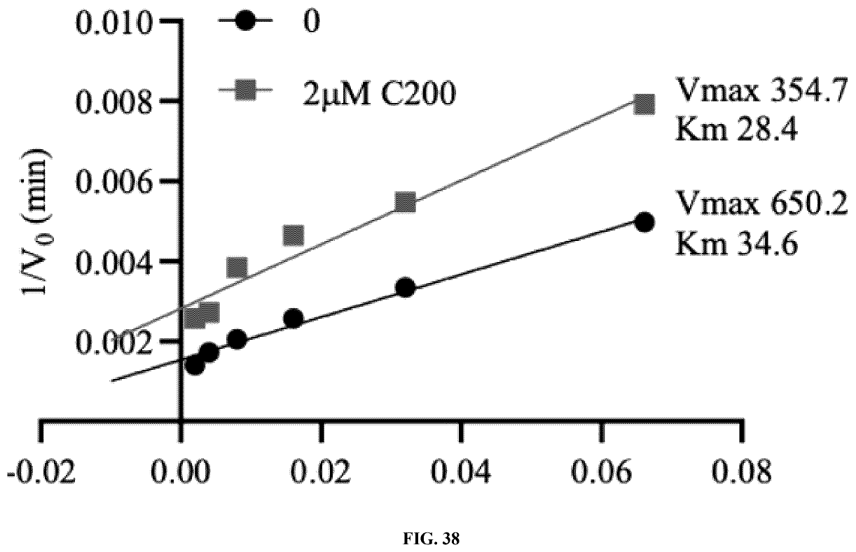

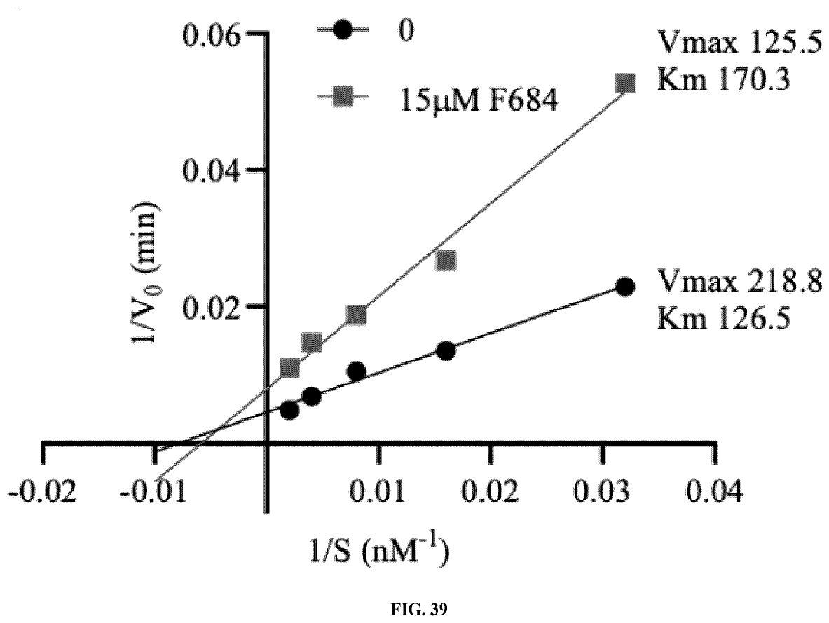

Subsequently, the inhibitory binding mechanism of compounds C200 and F684 was characterized via Michaelis-Menten modeling. The FRET-based EXO1 activity assay was performed at a fixed inhibitor concentration, which corresponded to the IC₅₀ of each compound (2 μM for C200 and 15 μM for F684), with varying substrate concentrations: 500 nM, 250 nM, 125 nM, 62.5 nM, 31.25 nM, and 15.625 nM (FIGS. 36 and 37 respectively). The initial velocity of enzyme activity was calculated from the rate within the linear region of the time dependent RFU. In the absence of an inhibitor, a Vmax=735.5 min−1 and Km=51.3 nM were observed. At 2 μM C200, Vmax was significantly decreased to 415.5 min⁻¹ while Km showed a reduction to 48 nM, suggesting an uncompetitive inhibition mechanism (FIG. 36). A similar trend of Vmax and Km reduction was also observed in an independent experiment with F684. Vmax was reduced from 292.9 min⁻¹ to 114.1 min⁻¹ upon addition of F684, while Km decreased from 227.6 nM to 145.5 nM (FIG. 37), which may be consistent with the improved binding and inhibitory capacity of F684 compared to C200 in other assays disclosed herein. Of note, although the baseline Vmax and Km values differed significantly between these two experiments, the effect of both compounds on Vmax and Km was consistent, these differences may be attributed to routine variances in purity and enzyme activity that can occur between batches. To confirm these changes in Vmax and Km for compound C200 and F684, Lineweaver-Burk plots were constructed by taking the double reciprocal of the Michaelis-Menten analysis and obtained a similar result (FIGS. 38 and 39).

The Michaelis-Menten and the Lineweaver-Burk plots both indicate that compounds C200 and F684 inhibit EXO1 activity through an uncompetitive mechanism. This suggests that both inhibitors interact with the EXO1:substrate complex at a region near the active site. Upon binding, compounds C200 and F684 can potentially trap EXO1 on the substrate and/or sterically block scissile bond alignment in the active site, consequently preventing catalysis from occurring. As such, while the affinity of EXO1 for the DNA substrate may be maintained, the portion of enzymes that are inhibitor-bound are “catalytically dead” or unable to cleave DNA.

### Example 6: Identification of Protein Residues that Direction Bind to Inhibitor Compounds

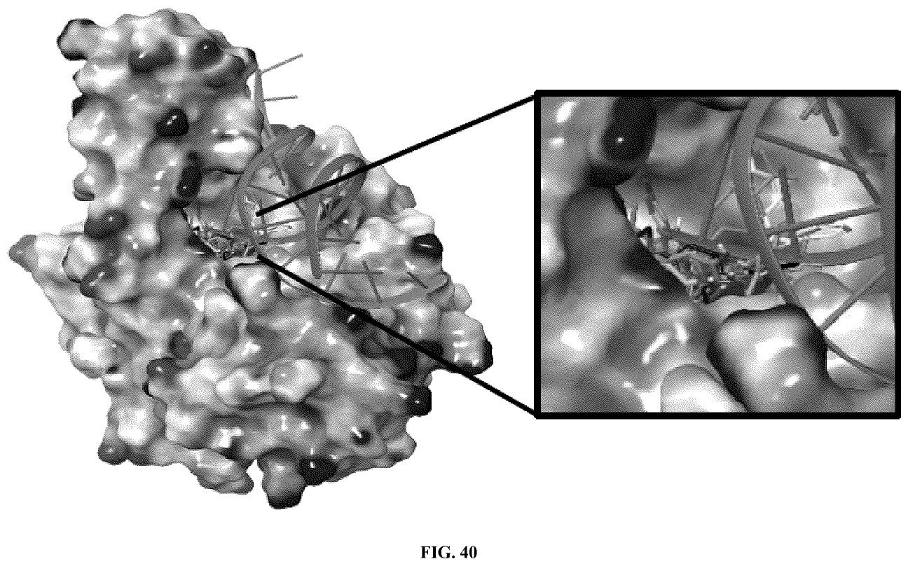

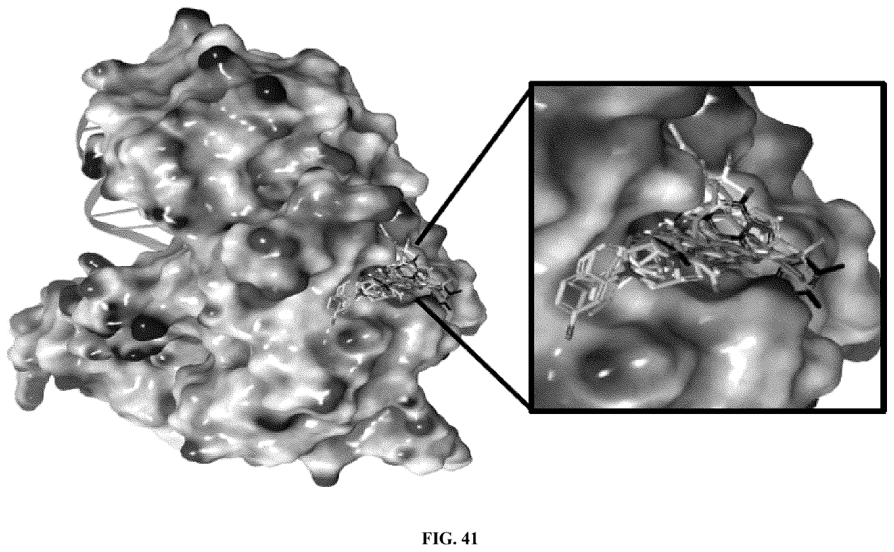

A three-dimensional structural model was utilized for virtual screening of small molecules that bind to EXO1. Druggable sites on the EXO1 model were predicted using an in-house-developed Druggable Site Prediction by FDA-approved drugs (DSP) methodology, which uses a diverse subset of 100 FDA-approved drug molecules to dock around the protein surface and predict the best inhibitor binding sites on the protein surface. The “best binding site” was defined as the protein pocket at which the highest number of the FDA-approved drugs were bound. We identified two potential inhibitor binding pockets for screening, designated as Sites 1 and 2. Site 1 is a well-defined pocket that is in close proximity to the DNA substrate (FIG. 40), and Site 2 is a shallow surface pocket at an allosteric site within the proximal end of the EXO1 C-terminal domain, in an area of unknown function (FIG. 41).

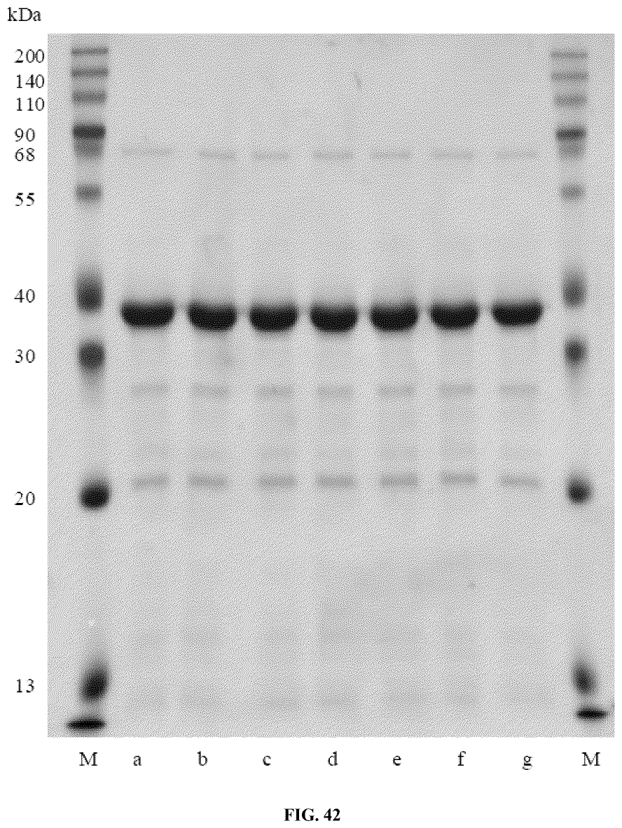

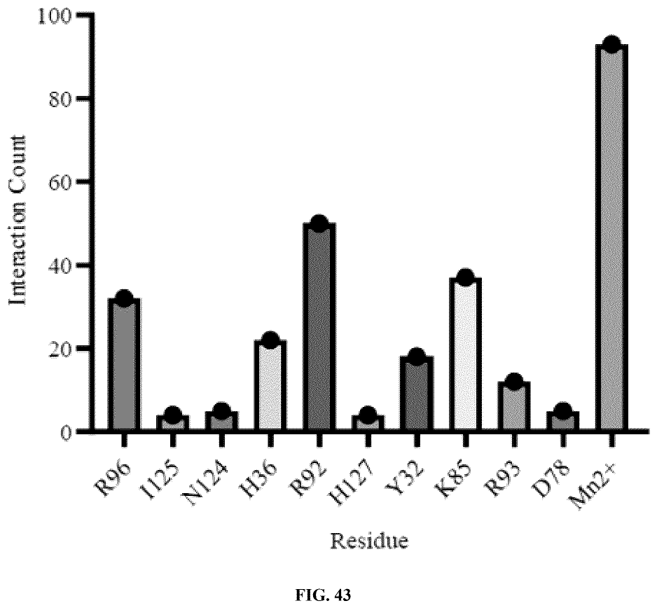

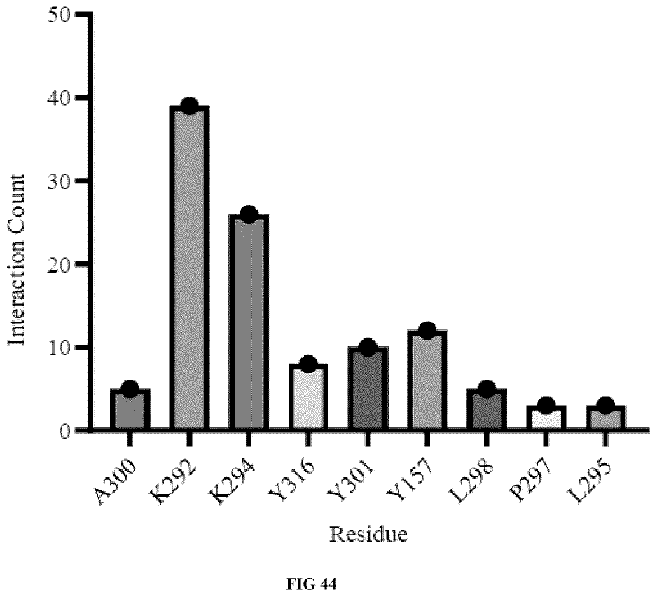

To validate the binding site of the inhibitor candidates, recombinant 6×His-tagged EXO1 N-terminal domain protein (residues 1-358 of SEQ ID NO:1; 38 kDa) with point mutations to residues K85, E89, R92, I125, or D225 in the Site 1 pocket, or dual mutation to K292 and K294 on the Site 2 surface were expressed and purified. SDS-PAGE was conducted with lanes marked M containing the pre-stained protein marker, (a) WT, (b) K85A, (c) E89A, (d) R92A, (e) I125A, (f) D225A, and (g) K292A-K294A recombinant proteins (bottom of figure) and the corresponding molecular weights indicated (left of figure) (FIG. 42). Each amino acid was mutated to alanine to minimize the potential of side chain interactions or interference. The amino acids were chosen based on a multi-structure induced-fit docking analysis to predict the specific residue interactions and potential conformational preference of our seven inhibitor scaffolds with EXO1 throughout its exonucleolytic reaction cycle. The six selected point mutations were chosen due to their frequent direct contact with the inhibitors across the molecular docking models (FIGS. 43 and 44). Briefly, the Y axis interaction count represents the total number of direct bonds between EXO1 and the experimental compounds found within the binding Site 1 (FIG. 43) and binding Site 2 (FIG. 44) including hydrogen bonds, pi stacking, and salt bridges, observed between individual amino acids (or active site metal ions) and the seven inhibitor scaffolds. The interaction count is based on induced-fit docking analysis across various conformations of EXO1 (PDBs: 5UZV, 5V04, 5V05, 5V06, 5V07, 5V08, 5V09, 5VOA, 5V0B). Residues which are known to be critical for EXO1 nuclease function were excluded from mutagenesis (i.e., Y32, H36).

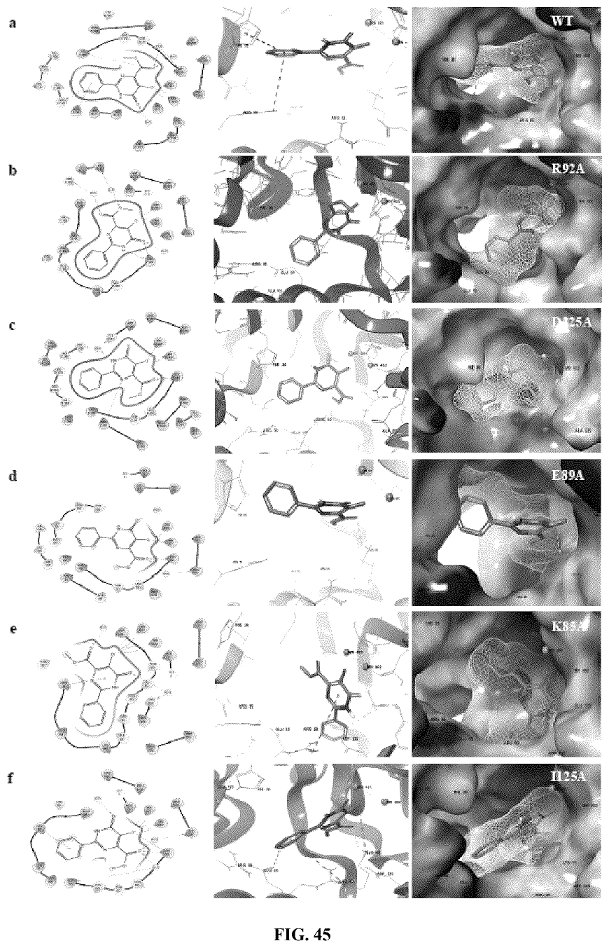

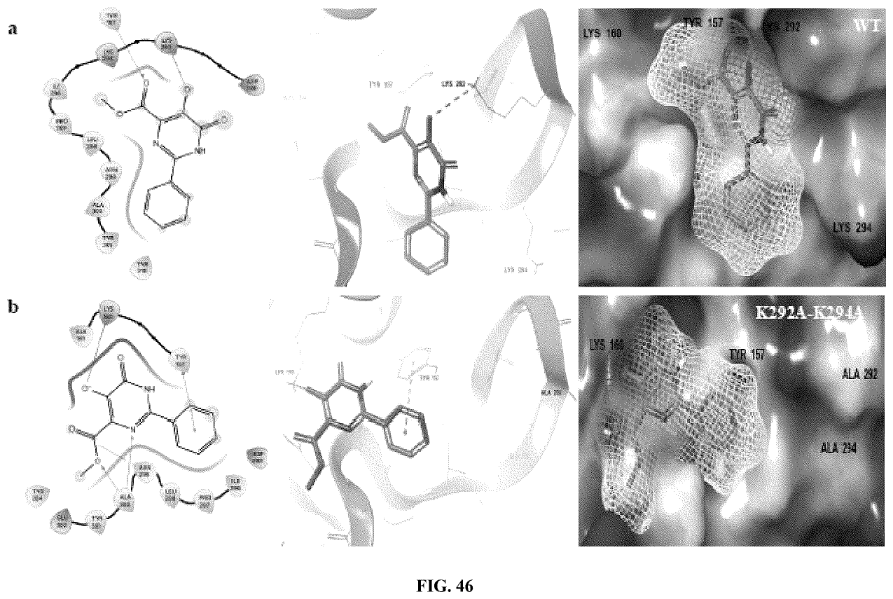

To further evaluate the impact of the selected Site 1 and Site 2-point mutations on inhibitor binding, compound C200 was docked onto models of the Site 1 and Site 2 mutants (PDB: 3QEB), and the bound positions and docking scores were compared to the corresponding wildtype protein model (FIGS. 45 and 46). FIG. 45 shows a comparison of the chemical bonds and position of compound C200 within the EXO1 Site 1 binding pocket before and after point mutations. FIG. 45, Left Panel displays all residues within 5 Å of C200; pink lines=hydrogen bonds (H-bonds), green lines=pi-pi stacking interactions, red lines=pi-cation interactions, and red-blue gradient lines=salt bridges. FIG. 45, Middle Panel displays the proximity of compound C200 in relation to the two catalytic metal ions within the active site. FIG. 45, Right Panel displays the electron density of the protein surface surrounding C200; red color indicates a negative charge, and blue indicates a positive charge. Direct bonds between inhibitor C200 and each of the EXO1 variants include: (a) EXO1-WT: H-bonding between C200, Lys85, and several active site waters; salt bridging to Mn2+ ions; and pi-pi stacking with His36 and Arg96. FIG. 45, Row b shows the EXO1-R92A interaction, demonstrating the H-bonding remained intact between compound C200, active site waters, Tyr32, and Lys 85, and salt bridging to Mn2+. However, both pi interactions were lost. FIG. 45, Row C shows the EXO1-D225A interaction, demonstrating the H-bonding between compound C200, Lys85, and active site waters, and salt bridging to Mn2+ remained. However, a new H-bond with Arg92 was gained, and both pi interactions were lost. FIG. 45, Row D shows the EXO1-E89A interaction, demonstrating the H-bonding was sustained between compound C200, active site waters, and Lys 85; as was salt bridging to Mn2+. Both pi interactions were lost. FIG. 45, Row E shows the EXO1-K85A interaction, demonstrating the H-bonding with active site waters remained, as did salt bridging to Mn2+. However, a new H-bond with Leu82, and pi stacking with Arg92 are observed. FIG. 45, Row F shows the EXO1-I125A interaction, demonstrating the H-bonding between compound C200, Lys85, and active site waters was retained, as well as salt bridging with Mn2+. A new pi interaction with Arg92 was observed. FIG. 46 shows how the binding position of compound C200 changes upon mutation of the Site 1 binding pocket, with the color of compound C200 corresponding to the ribbon color for each EXO variant as displayed in FIG. 45. Comparison of the binding position and interactions of compound C200 within the EXO1 Site 2 binding pocket before and after point mutations. FIG. 46, Left Panel displays all residues within 5 Å of the compound; pink lines=H-bonds, green lines=pi-pi stacking interactions, red lines=pi-cation interactions, and red-blue gradient lines=salt bridges. FIG. 46, Middle Panel displays the proximity of compound C200 to the two catalytic metal ions within the EXO1 active site. FIG. 46, Right Panel displays the electron density of the protein surface surrounding compound C200; red color indicates negative charge and blue indicates positive charge. Chemical bonds between compound C200 and EXO1 Site 2 variants were investigated. FIG. 46, Row A shows the EXO1-WT interaction, demonstrating the H-bond is observed with Tyr157, and a salt bridge to Lys292. FIG. 46, Row B shows the EXO1-K292A-K294A interaction, demonstrating the H-bond between Tyr157 is converted to a pi stacking interaction, and salt bridging has been transferred to Lys160. H-bonds with Asn299 and Ala300 are also gained. In vitro, compound C200 activity was lost upon K292A-K294A mutation, supporting Site 2 as an alternative binding site for EXO1 inhibitors.

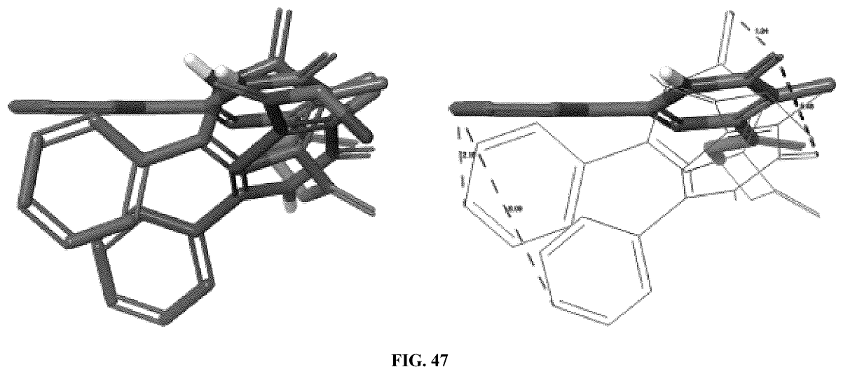

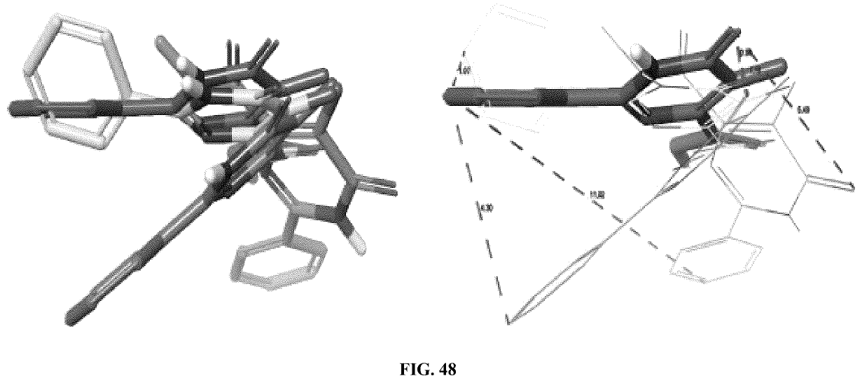

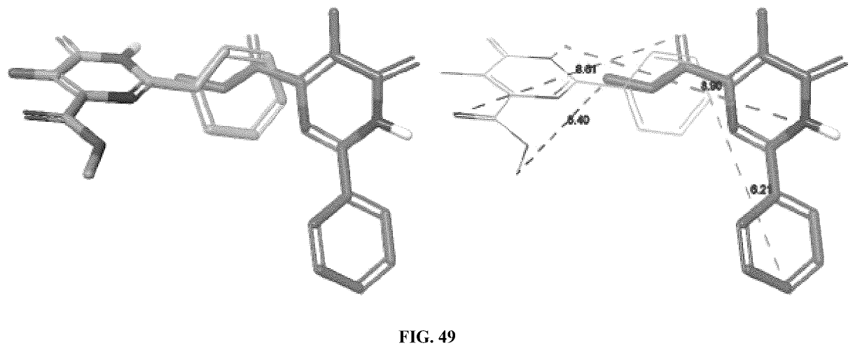

It was discovered that among the Site 1 mutants, R92A and D225A mutants resulted in a significant change in compound C200 binding position. FIG. 47 demonstrates the EXO1-R92A and EXO1-D225A mutations resulted in a 6·09 Å (blue compound) and 2·16 Å (purple compound) deviation of compound C200's benzene ring from EXO1-WT (red compound). For both mutants, the benzene and pyrimidine rings of compound C200 are rotated >45°, significantly changing compound C200's position between 2 and 8 Å compared EXO1-WT. These positional changes may corresponded with a loss of compound C200 inhibitory activity, measured via in vitro FRET-based assay. The distance between the binding positions of compound C200 in the R92A, D552A, and WT are indicated by pink dotted lines and the corresponding values. In contrast, the K85A, E89A, I125A mutants showed minimal change to compound C200 binding position (FIG. 48). FIG. 48 shows changes in compound C200 binding position within the Site 1 pocket of EXO1-WT (red compound), EXO1-E89A (yellow compound), EXO1-K85A (teal compound), and EXO1-I125A (magenta compound). The E89A mutation slightly displaces the benzene ring of C200 by <90°, resulting in a modest positional deviation of ~1·0 Å. In contrast, EXO1-K85A mutation resulted in a similar positioning of compound C200 to EXO1-WT, except with a ~90° clockwise rotation changed its overall position from 5·49 to 11·02 Å. Similarly, compound C200 binding against EXO1-I125A showed a ~45° downward shift of the benzene ring while the oxygen groups remain relatively unchanged, resulting in a lopsided positional change from EXO1-WT that ranged from 4·30 Å at the benzene to 1·16 Å at the opposing end. E89A, I125A, K85A mutants did not have a significant impact on compound C200 inhibitory activity in vitro, suggesting that positioning of the pyrimidine group of compound C200 in proximity to the metal ions may be critical to EXO1 inhibition. Distance measurements of each mutant from EXO1-WT are indicated by pink dotted lines and values. As for Site 2, mutation of K292A and K294A resulted in a deviated C200 pose with distance measurements from the wildtype docking position as shown in FIG. 49. For example FIG. 49 shows the changes in compound C200's binding position that occur when the Site 2 binding pocket is mutated. The color of the C200 compound in FIG. 49 corresponds to the ribbon color for each EXO variant as displayed in FIG. 46. The EXO1-K292A-K294A mutation caused a 6 to 8 deviation of C200 (orange compound) compared to EXO1-WT (red compound) (FIG. 49). This positional change corresponded with the loss of C200 inhibitory activity by FRET assay, suggesting that strong binding to Tyr157 via H-bonds may be essential for EXO1 inhibition via Site 2. Individual docking scores for C200 and F684 against each mutant are listed in Table 4.

TABLE 4
Docking scores (kcal/mol) of C200 against EXO1 mutants

| | Rigid Score | Induced-Fit Score |
|---|---|---|
| C200 | | |
| Site 1 | | |
| WT | −6.620 | −781.70 |
| K85A | −7.489 | −15691.59 |
| E89A | −6.777 | −761.33 |
| R92A* | −5.531 | −785.21 |
| I125A | −6.544 | −15615.61 |
| D225A* | −5.491 | −15754.60 |
| Site 2 | | |
| WT | −2.334 | −770.85 |
| K292A/K294A* | −2.364 | −770.55 |
| F684 | | |
| Site 1 | | |
| WT | −11.448 | −15218.55 |
| K85A* | −8.900 | −15091.50 |
| E89A | −9.775 | −15080.40 |
| R92A* | −10.375 | −15091.14 |
| I125A | −11.110 | −15155.45 |
| D225A | −11.145 | −15100.80 |
| Site 2 | | |
| WT | −5.209 | −747.77 |
| K292A/K294A | −4.066 | −741.35 |

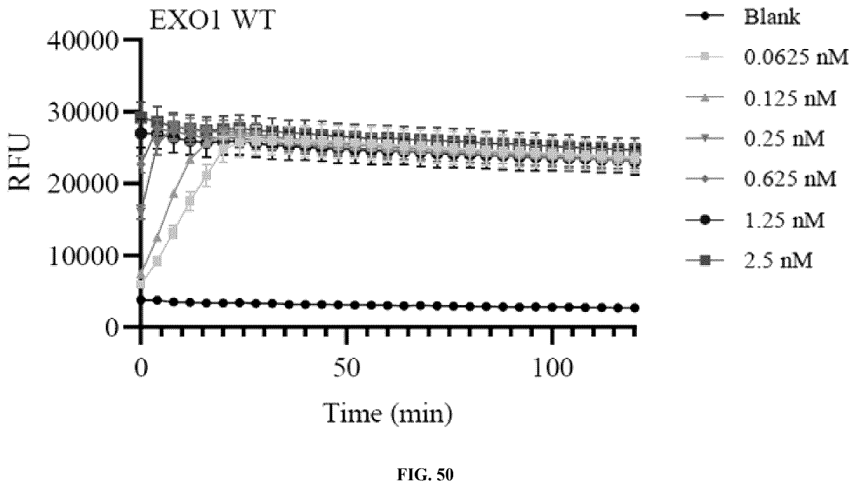

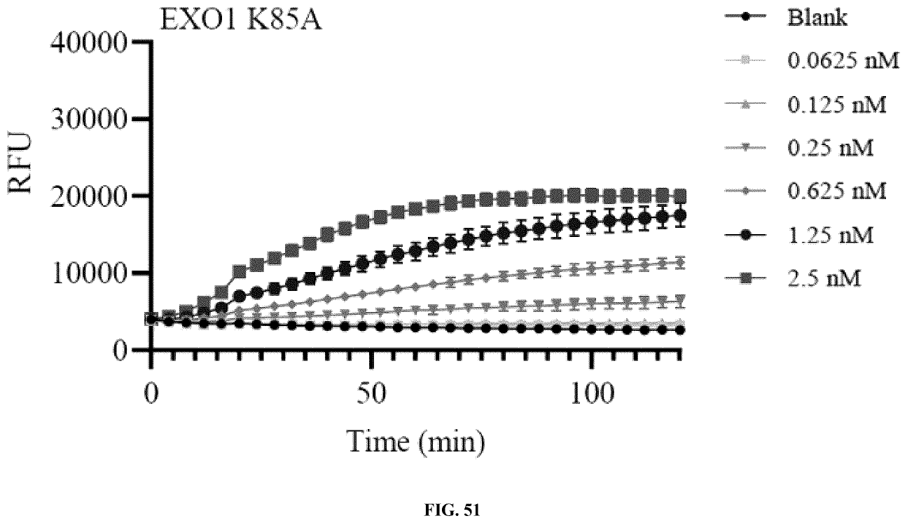

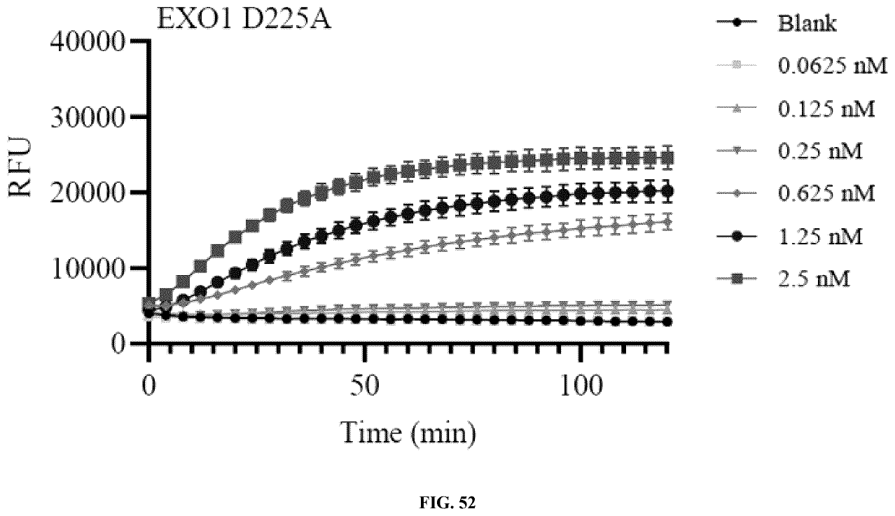

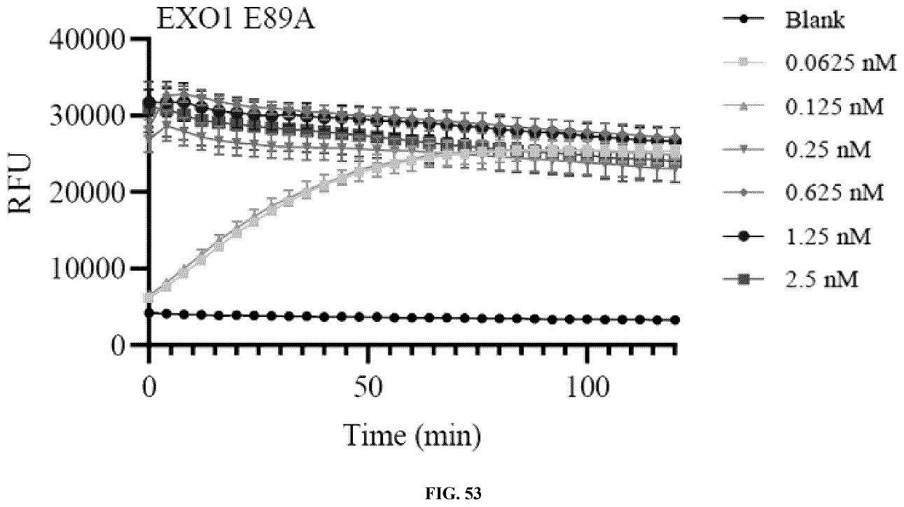

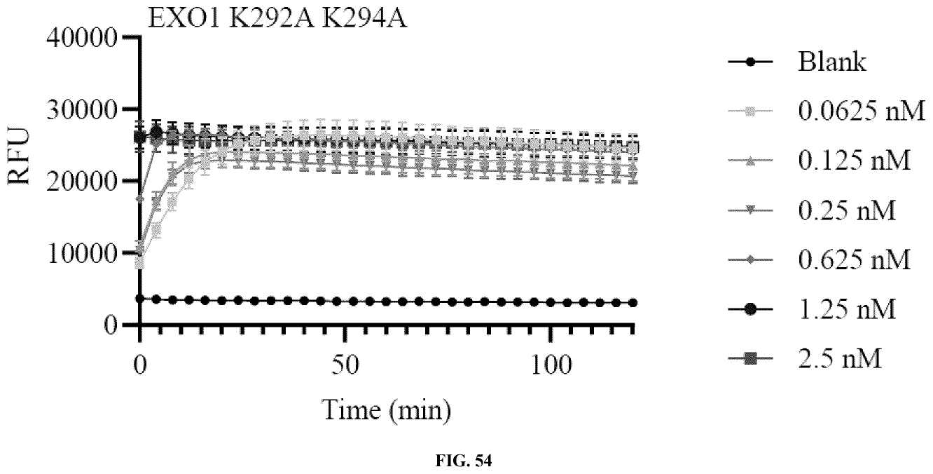

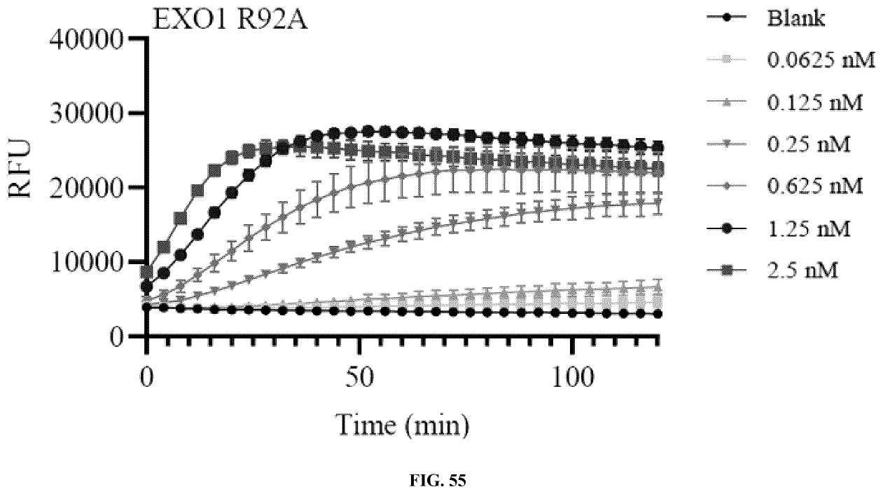

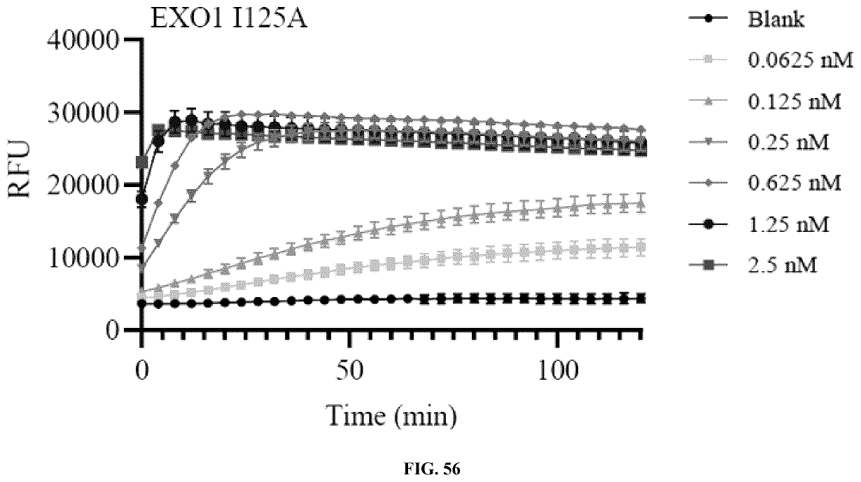

Simultaneously, the effect of each point mutation on in vitro exonuclease activity was investigated by comparing the enzymatic activity of EXO1 before and after point mutations to Site 1 and Site 2 binding pockets (FIGS. 50-56). FIG. 50 shows the EXO1-WT interaction, demonstrating full activity, max. signal=31,125 RFU. FIG. 51 shows the EXO1-K85A interaction, demonstrating a moderate loss of activity, max. signal=20,841 RFU. FIG. 52 shows the EXO1-D225A interaction, demonstrating a moderate loss of activity, max. signal=25,543 RFU. FIG. 53 shows the EXO1-E89A interaction, demonstrating a full activity, max. signal=34,328 RFU. FIG. 54 shows the EXO1-K292AK294A interaction, demonstrating a minimal loss of activity, max. signal=28,651 RFU. FIG. 55 shows the EXO1-R92A interaction, demonstrating a minimal loss of activity, max. signal=27,015 RFU. FIG. 56 shows the EXO1-I125A interaction, demonstrating a minimal loss of activity, max. signal=30,483 RFU. Most mutations in Site 1 result in some loss of enzymatic activity compared to WT EXO1 (FIG. 50), particularly K85A (FIG. 51) and D225A (FIG. 52). However, E89A (FIG. 53) and Site 2 mutation K292A-K294A (FIG. 54) showed only minimal loss of activity.

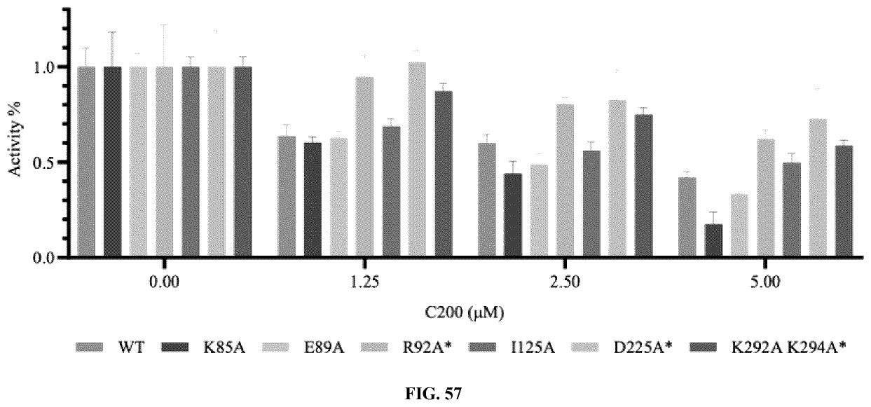

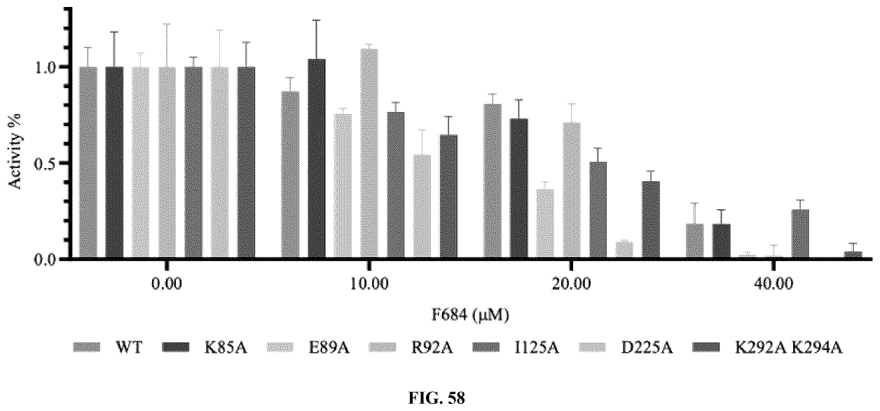

The activity of each mutant protein with inhibitors C200 and F684 were then tested. The EXO1 enzyme concentration was 25 nM, DNA substrate concentration was 250 nM. It was discovered that compound C200 effectively blocked the exonuclease activity of the R92A, D225A, and K292A-K294A mutants at compound C200 concentrations ranging from 0.0 to 5.0 μM (FIG. 57). Compound F684 was also investigated with concentrations ranging from 10 to 40 M. DMSO without inhibitors was used as a control and its relative binding activity was set equal to 1 (100%). Compound F684 had a moderate reduction in inhibitor effect against R92A and K85A mutants at low concentrations (i.e., 10 μM concentration), however at concentrations of 20 and 40 μM, the mutants (i.e., K85 Å and R92A) (FIG. 58). The values shown are the mean±s.d. of three independent experiments for both FIGS. 57 and 58. This data suggests that the inhibitory activity of C200 and F684 are primarily mediated by interaction with EXO1 residue R92 and other nearby residues in Site 1, which are in close proximity to catalytic metal ions within the active site of the protein. It may also implies that, at least in the case of compound C200, the compound may additionally be capable of blocking EXO1 nuclease activity via allosteric interaction with Site 2.

### Example 7: Analysis of Double Strand Breaks and S-Phase Parylation after Treatment with Inhibitor Compounds

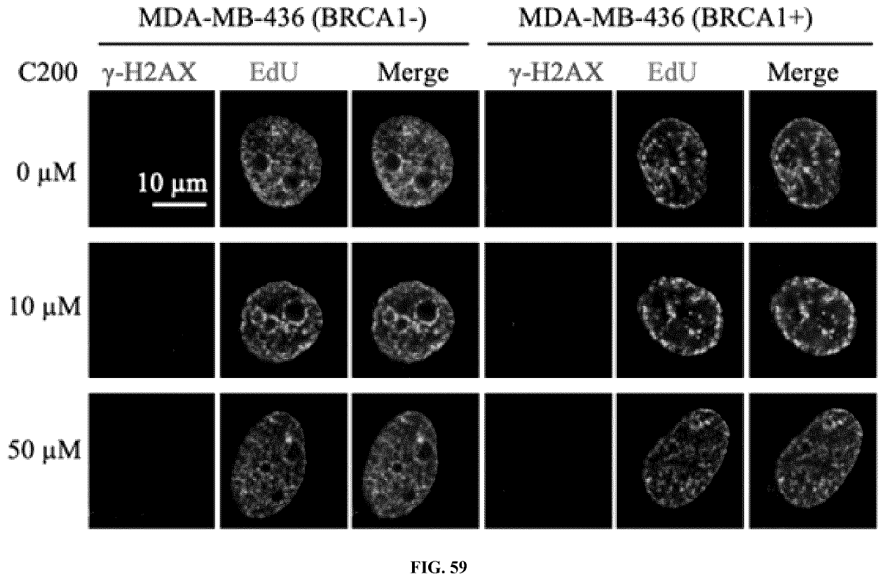

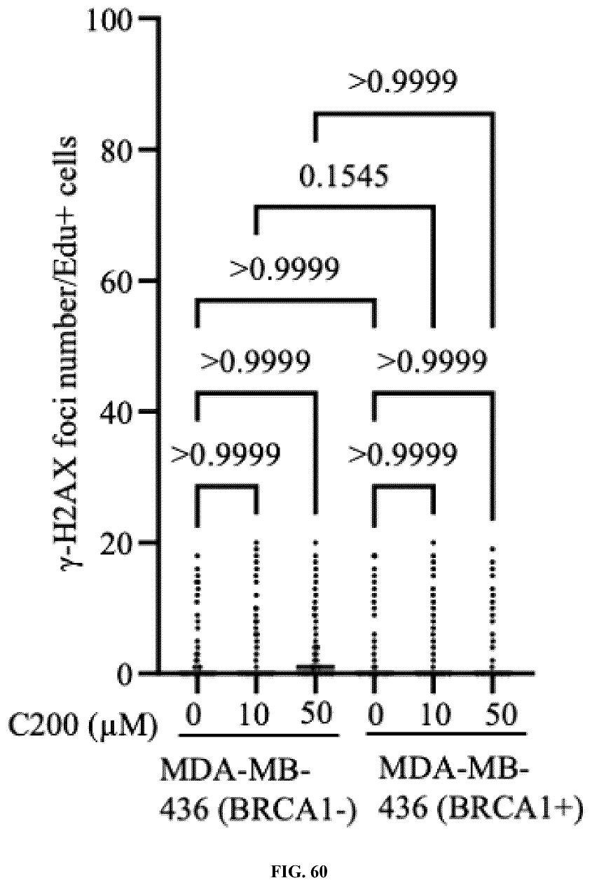

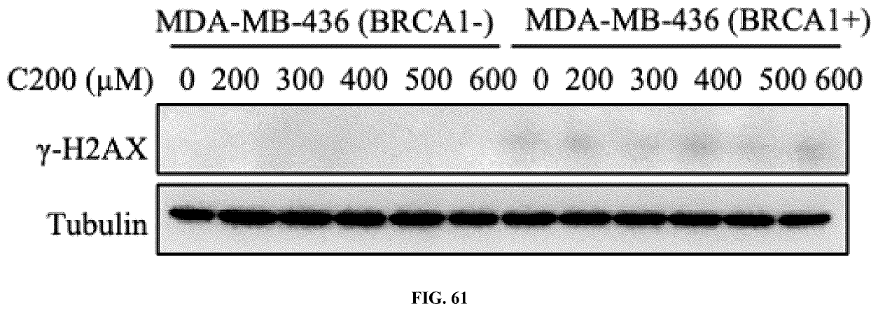

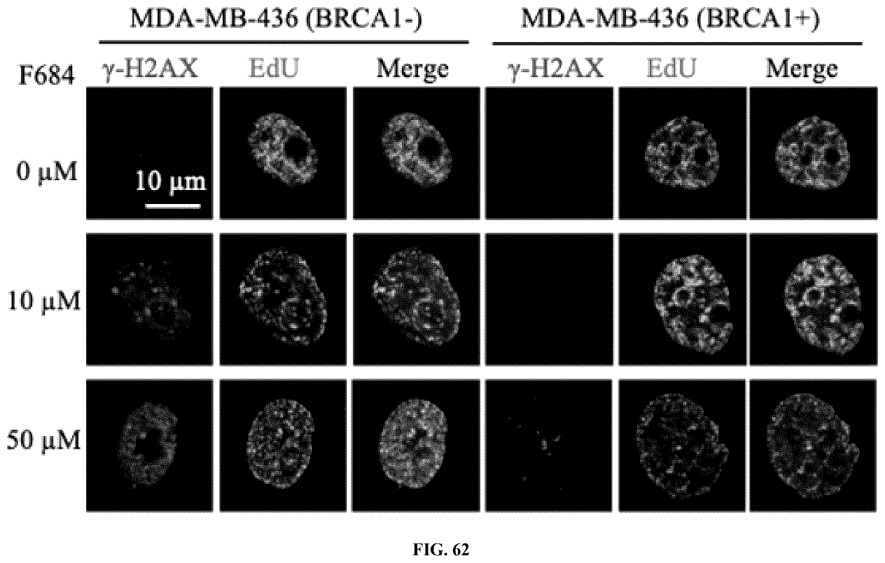

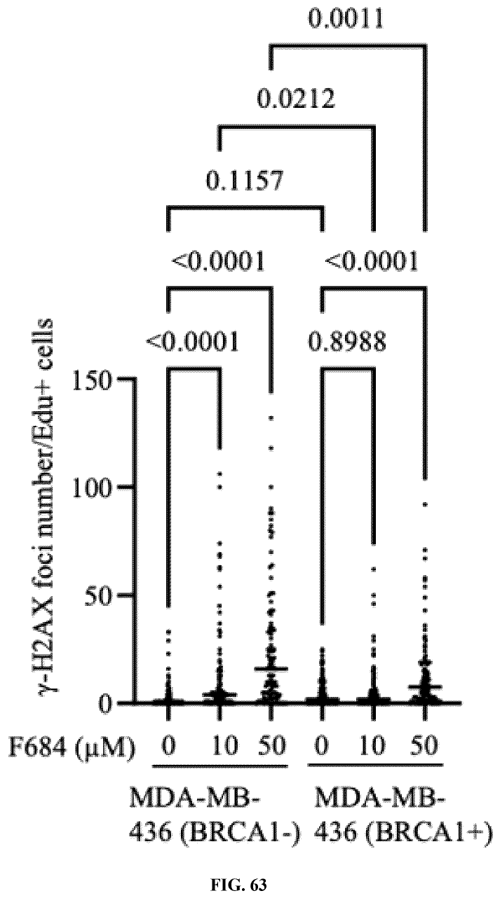

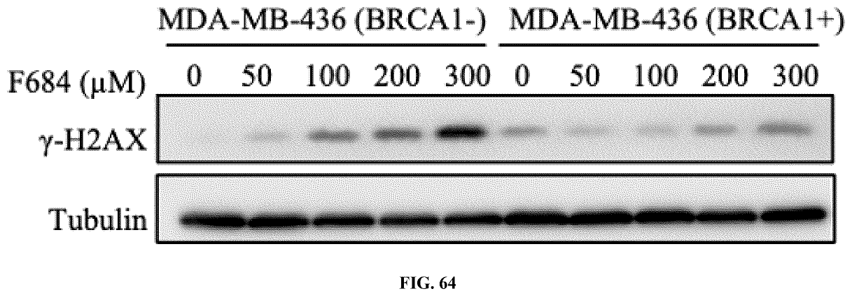

To investigate whether unresolved double strand breaks (DSBs) are involved in the synthetic lethal interaction between BRCA1 deficiency and the described EXO1 inhibitors, γ-H2AX levels were analyzed as a marker for unresolved DSBs. FIG. 59 demonstrates EXO1 inhibition by compound C200 did not cause a significant increase in DSB burden. Briefly, parental MDA-MB-436 (BRCA1−) and WT BRCA1-expressing MDA-MB-436 (BRCA1+) cells were untreated or treated with 10 μM or 50 μM C200 overnight. A minimal quantity of γ-H2AX foci was observed; while EdU incorporation shows active DNA replication. FIG. 60 shows the quantification of the relative γ-H2AX per nucleus from FIG. 59. FIG. 61 shows a western blot analysis of γ-H2AX after increasing concentrations of compound C200 treatment. The intensity of γ-H2AX bands correlates with the level of DSBs observed in the immunofluorescence images in FIG. 59. FIG. 62 demonstrates EXO1 inhibition by F684 significantly increased DNA damage. Briefly, parental MDA-MB-436 (BRCA1−) and WT BRCA1-expressing MDA-MB-436 (BRCA1+) cells were untreated or treated with 10 μM or 50 μM F684 overnight. Strong γ-H2AX foci were observed, indicating an increased burden of DSBs. EdU incorporation shows active DNA replication, and partial co-localization with γ-H2AX, suggesting the DNA damage may be replication-associated. FIG. 63 shows the quantification of the relative γ-H2AX per nucleus depicted in FIG. 62. FIG. 64 shows a western blot analysis of γ-H2AX after increasing concentrations (i.e., 0 to 300 μM) of F684 treatment. The intensity of γ-H2AX bands correlates with the level of DSBs observed in the immunofluorescence images in FIG. 62. In summary, in MDA-MB-436 parental cells, BRCA1-deficiency combined with inhibitor compound C200 had no effect on γ-H2AX levels (FIGS. 59-61) whereas treatment with F684 elicited a significant increase in γ-H2AX after 24 h (FIGS. 62-64).

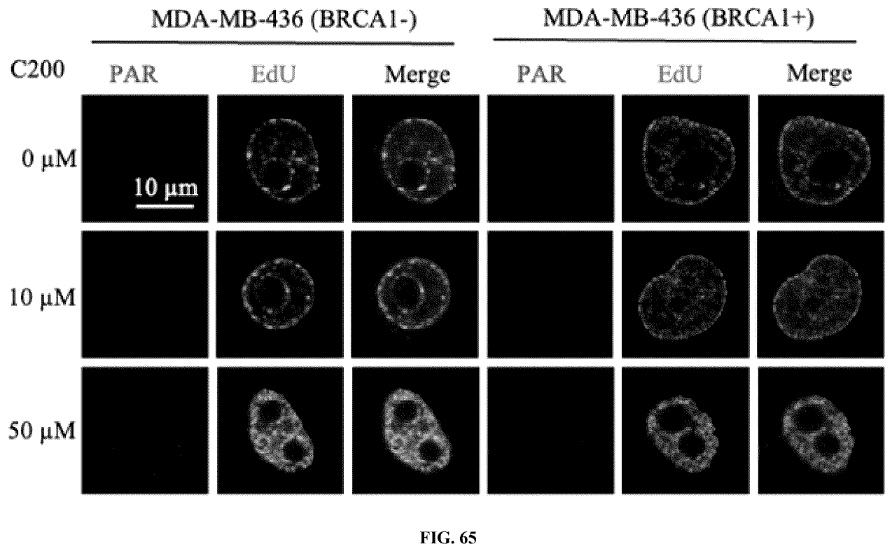

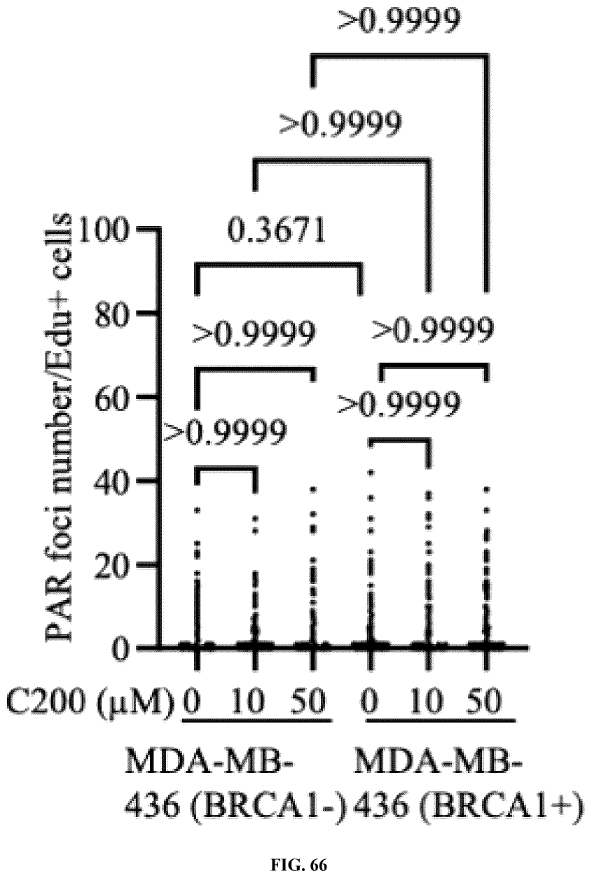

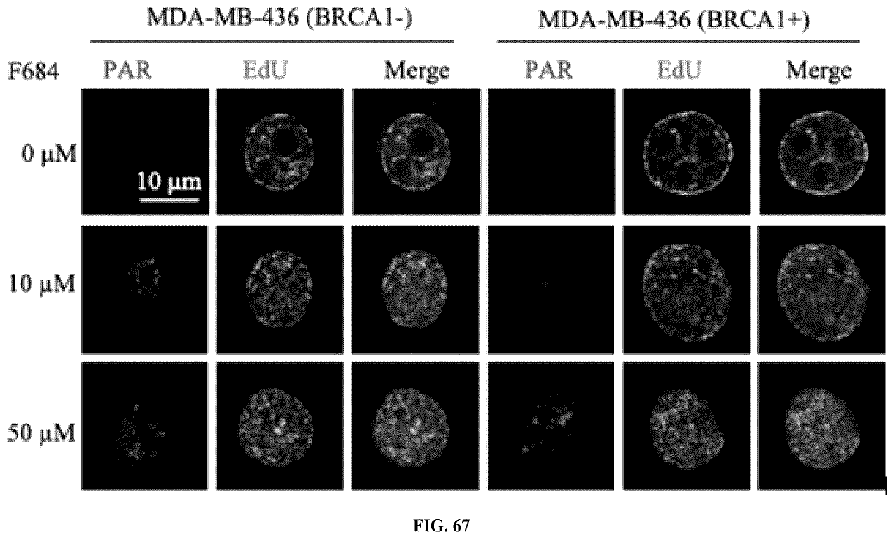

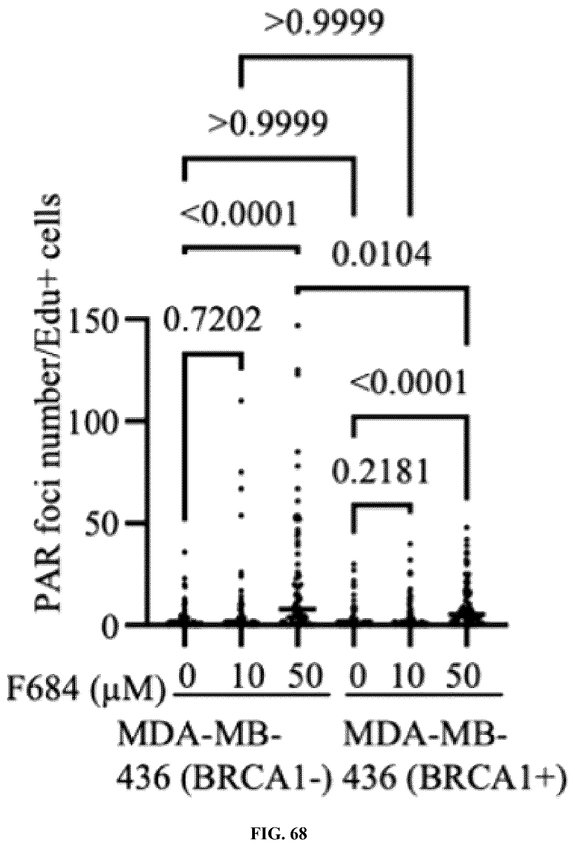

Additionally, the S-phase Poly(ADP-ribosyl)ation (PARylation) was measured, which has been reported to reflect the presence of ssDNA gaps, and more particularly, unprocessed Okazaki fragments, although it is also induced at DSBs and other lesions. FIG. 65 demonstrates EXO1 inhibition by compound C200 did not significantly increase PARylation. Briefly, parental MDA-MB-436 (BRCA1−) (FIG. 65, Panel A) and WT BRCA1-expressing MDA-MB-436 (BRCA1+) (FIG. 65, Panel B) cells were untreated or treated with 10 μM or 50 μM compound C200 overnight. Low PARylation foci levels were observed (FIG. 65, Panel A&B, left column), while EdU incorporation shows active DNA replication (FIG. 65, Panel A&B, middle column). FIG. 66 shows the quantification of the relative PARylation per nucleus from FIG. 65. FIG. 67 demonstrates EXO1 inhibition by F684 significantly increased PARylation. Briefly, parental MDA-MB-436 (BRCA1−) (FIG. 67, Panel A) and WT BRCA1-expressing MDA-MB-436 (BRCA1+) (FIG. 67, Panel B) cells were untreated or treated with 10 μM or 50 μM F684 overnight. Strong PARylation foci are observed in BRCA1− cells (FIG. 67, Panel A&B, left column). EdU incorporation shows active DNA replication, and partial co-localization with PARylation suggests the DNA damage is replication-associated (FIG. 67, Panel A&B, middle column). FIG. 68 shows the quantification of the relative PARylation per nucleus from FIG. 67.

In summary, similar to the previous analysis, no effect on S-phase PARylation when EXO1 was inhibited by compound C200 in MDA-MB-436 cells was observed, regardless of BRCA1 status (FIGS. 65 and 66), whereas an additive effect on S-phase PARylation was observed with compound F684, particularly in MDA-MB-436 parental (BRCA1−) cells (FIGS. 67 and 68). These results support a potential synthetic lethal relationship between EXO1 inhibitor F684 and BRCA1-deficiency due to an increased burden of unrepaired DSBs and unligated Okazaki fragments.

### Example 8: Inhibitors Block DNA End Resection

It was investigated whether or not it could be verified if the defects in DSB repair were due to inhibition of end resection. To measure end resection, the level of phosphorylated RPA2 (S33 or S4/8) in MDA-MB-231 cells treated with compounds C200 and F684 in the presence or absence of camptothecin (CPT) was determined. CPT stabilizes cleavable complex intermediates in topoisomerase I reactions, which collapse into DSBs when encountered by a replication fork. CPT increased phosphorylated RPA2 (P-RPA), as measured on western blots.

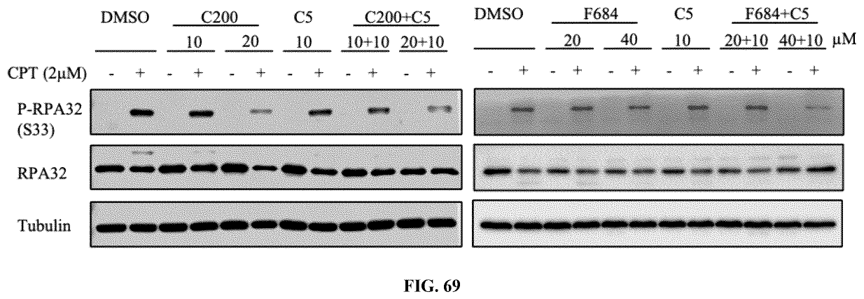

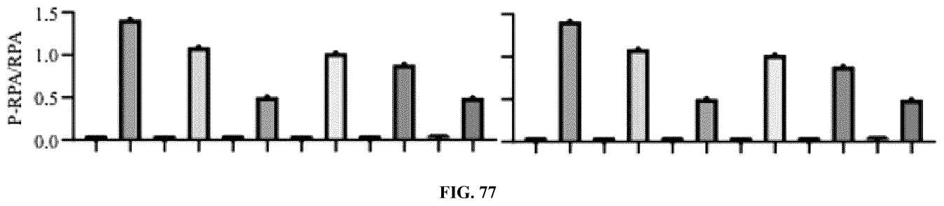

FIG. 69 shows EXO1 inhibition by compounds C200 or F684 impaired replication fork-related DNA end resection in MDA-MB-231 breast cancer cells at similar levels. Briefly, MDA-MB-231 cells were left untreated or treated with 10 μM or 20 μM C200 for overnight (FIG. 69, left panels), or left untreated or treated with 20 μM or 40 μM F684 for overnight (FIG. 69, right panels), or combined with 10 μM C5. The cells were then treated with 2 μM CPT for 2 h. The levels of RPA32 and phosphorylated RPA32 (S33) were analyzed by western blot using antibodies against RPA32 and phosphorylated RPA32 (S33). Total level of tubulin was used as a control. Treatment with F684 or C200 significantly reduced the CPT-induced P-RPA levels (FIG. 77); and consistent with our previous findings, DNA2 inhibitor C5 also demonstrated a significant reduction in the CPT-induced P-RPA level which synergized with F684 treatment (FIG. 69).

### Example 9: Inhibitor Compound F684 Reduces Double Strand Break Repair Capacity

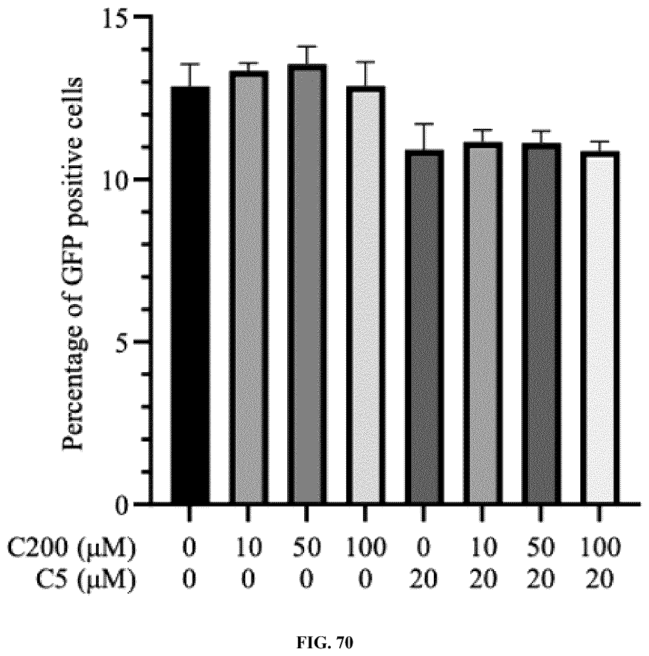

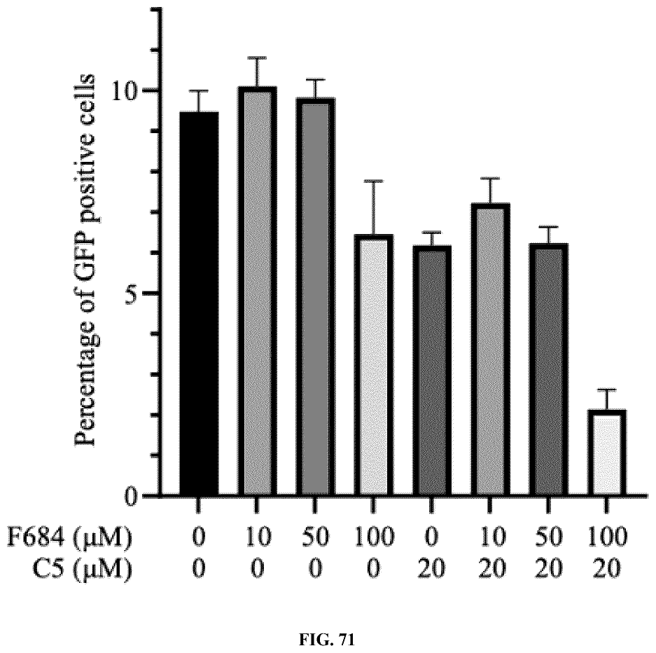

To determine if the reduction in DNA end resection had an effect on the frequency of HR, EXO1 inhibitor treatment was assessed in U2OS cells harboring GFP-based reporters. Cells were infected with adenovirus expressing I-SceI restriction enzyme to induce site-specific DNA DSBs and treated with 0, 10, 50, or 100 μM of compound C200, either alone or in combination with the DNA2 inhibitor C5 (10 μM). Consistent with previous findings, flow cytometry analysis of GFP-positive cells 48 h post-treatment revealed no significant changes in HR frequencies across the compound C200 treatment groups compared to the untreated control (FIG. 70). These findings suggest that compound C200, even at higher concentrations, does not impair the repair efficiency of the HR pathway. Next, U2OS cells were treated in an analogous manner, with 0, 10, 50, or 100 M of compound F684 and with or without C5. In contrast to compound C200, it was discovered that treatment with compound F684 resulted an inhibition of HR that increased with dose, particularly when combined with C5. Flow cytometry showed a marked reduction in GFP-positive cells at 100 M compound F684, indicating significant suppression of the HR pathway; and, the inhibitory effect of compound F684 was further enhanced when combined with C5 (FIG. 71). These results indicate that compound F684 binding successfully disrupts cellular DSB repair by HR. Values are mean±s.d. of three independent experiments.

### Example 10: Tumor Cell Profiling Reveals Micromolar Cytotoxicity Across Multiple Cancer Types and Synergy with BRCA1 Deficiency In Vitro and In Vivo

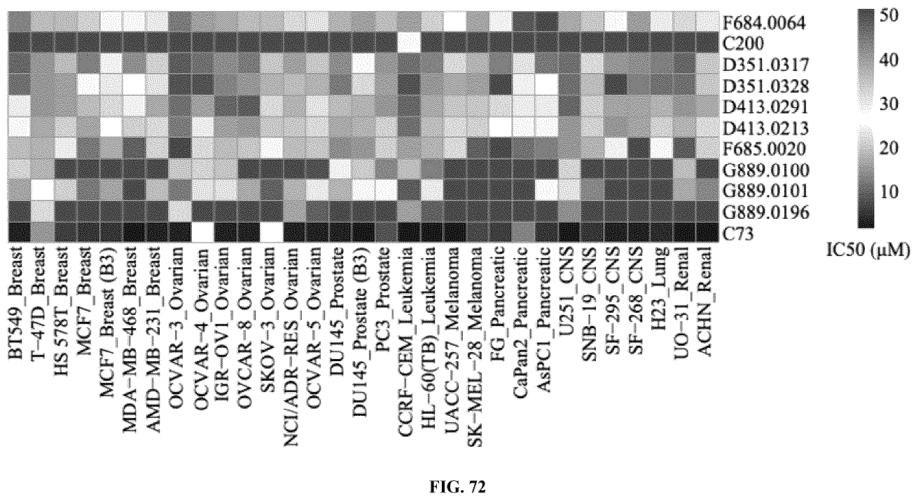

A subset of the NCI-60 cell line panel supplemented with an additional three pancreatic cell lines (FG, AsPC1, CaPan2), were tested. Briefly, IC₅₀ s of representative compounds from six of the seven EXO1 inhibitor scaffolds across 31 cancer cell lines compared to previously reported EXO1 inhibitor, C73. Blue color indicates potency <25 μM, white color indicates potency ~25 μM, and red color indicates potency >30 μM. Inhibitor compound C200 showed little efficacy in killing cells as the IC₅₀ s were greater than 50 μM, compound F684 was more effective with IC₅₀ s generally in the 15-25 μM range (Table 5 and FIG. 72).

TABLE S5
NCI-60 cell line panel IC₅₀s

| F684- | | D351- | D351- | D413- | D413- | F685- | G889- | G889- | G889- | |
|---|---|---|---|---|---|---|---|---|---|---|
| 0064 | C200 | 0317 | 0328 | 0291 | 0213 | 0020 | 0100 | 0101 | 0196 | C73 |

Breast

| | | | | | | | | | | | |
|---|---|---|---|---|---|---|---|---|---|---|---|
| BT549 | 11.95 | >50.00 | 8.64 | 9.76 | 23.37 | 23.82 | 17.88 | 31.48 | 37.81 | >50.00 | 1.73 |
| T-47D | 17.97 | >50.00 | 13.91 | 13.72 | 13.70 | 16.42 | 16.86 | 36.25 | 25.39 | 30.78 | 13.76 |
| HS 578T | 18.19 | >50.00 | 17.57 | 15.33 | 15.45 | 20.11 | 22.93 | >50.00 | 19.92 | >50.00 | 5.29 |
| MCF7 | 20.08 | >50.00 | 43.29 | 27.24 | 18.02 | 37.79 | 43.52 | >50.00 | 43.56 | >50.00 | 4.50 |
| MCF7 (B3) | 23.59 | >50.00 | 28.25 | 28.44 | 21.43 | 25.17 | 37.27 | >50.00 | 37.46 | >50.00 | 3.84 |
| MDA-MB-468 | 23.62 | >50.00 | 40.46 | 25.86 | 27.65 | 20.00 | 46.35 | >50.00 | >50.00 | >50.00 | 0.83 |
| MDA-MB-231 | 28.56 | >50.00 | 19.66 | 21.50 | 16.05 | 21.29 | 31.59 | >50.00 | 35.19 | >50.00 | 2.27 |

Ovarian

| | | | | | | | | | | | |
|---|---|---|---|---|---|---|---|---|---|---|---|
| OVCAR-3 | 10.93 | >50.00 | 7.59 | 7.38 | 9.76 | 10.97 | 5.73 | 18.69 | 10.85 | 31.06 | 2.18 |
| OVCAR-4 | 15.38 | >50.00 | 10.29 | 7.14 | 15.38 | 23.36 | 21.42 | 31.74 | 21.54 | >50.00 | 24.97 |
| IGR-OV1 | 16.24 | >50.00 | 12.99 | 12.51 | 9.28 | 14.95 | 18.09 | 34.21 | 29.49 | >50.00 | 2.13 |
| OVCAR-8 | 18.12 | >50.00 | 10.80 | 14.24 | 8.51 | 13.63 | 18.77 | >50.00 | 16.30 | >50.00 | 1.88 |
| SKOV-3 | 18.46 | >50.00 | 19.28 | 17.16 | 20.95 | 18.86 | 24.36 | >50.00 | 46.46 | >50.00 | 24.68 |
| NCI/ADR-RES | 20.83 | >50.00 | 15.44 | 17.94 | 16.86 | 19.14 | 17.70 | >50.00 | 17.23 | 37.73 | 1.91 |
| OVCAR-5 | 21.58 | >50.00 | 18.51 | 17.29 | 21.60 | 21.38 | 19.36 | 48.59 | 29.17 | 47.27 | 1.75 |

Prostate

| | | | | | | | | | | | |
|---|---|---|---|---|---|---|---|---|---|---|---|
| DU145 | 16.72 | 49.37 | 12.83 | 11.52 | 18.03 | 15.96 | 12.57 | 27.99 | 35.93 | >50.00 | 2.42 |
| DU145 (B3) | 15.38 | >50.00 | 27.12 | 21.32 | 16.69 | 16.85 | 14.09 | 33.30 | 24.14 | >50.00 | 1.09 |
| PC3 | 18.02 | >50.00 | 14.13 | 14.53 | 14.28 | 19.77 | 19.74 | 41.72 | 26.58 | >50.00 | 6.60 |

Leukemia

| | | | | | | | | | | | |
|---|---|---|---|---|---|---|---|---|---|---|---|
| CCRF-CEM | 14.64 | 27.40 | 10.32 | 7.02 | 9.16 | 9.84 | 14.82 | 34.69 | 8.14 | 15.32 | 0.61 |
| HL-60(TB) | 20.10 | >50.00 | 12.17 | 14.03 | 15.87 | 18.05 | 17.47 | 43.15 | 28.45 | 46.78 | 0.76 |

Melanoma

| | | | | | | | | | | | |
|---|---|---|---|---|---|---|---|---|---|---|---|
| UACC-257 | 24.96 | >50.00 | 20.48 | 16.07 | 19.22 | 20.77 | 21.46 | 50.27 | 49.56 | >50.00 | 1.47 |
| SK-MEL-28 | 37.96 | >50.00 | 16.52 | 15.11 | 19.05 | 20.36 | 47.14 | >50.00 | >50.00 | >50.00 | 6.10 |

Pancreatic

| | | | | | | | | | | | |
|---|---|---|---|---|---|---|---|---|---|---|---|
| FG | 27.54 | >50.00 | 43.09 | >50.00 | 20.75 | 25.52 | >50.00 | >50.00 | >50.00 | >50.00 | 5.34 |
| CaPan2 | 48.09 | >50.00 | 30.22 | 28.15 | 22.47 | 23.64 | 44.69 | >50.00 | >50.00 | >50.00 | 12.47 |
| AsPC1 | >50.00 | >50.00 | 43.44 | 25.38 | 22.79 | 23.80 | 43.28 | >50.00 | 27.04 | >50.00 | 4.11 |

Central Nervous System

| | | | | | | | | | | | |
|---|---|---|---|---|---|---|---|---|---|---|---|
| U251 | 16.17 | >50.00 | 9.79 | 9.64 | 9.34 | 13.64 | 14.22 | 31.67 | 30.21 | 40.68 | 0.77 |
| SNB-19 | 19.34 | >50.00 | 20.79 | 17.97 | 19.90 | 19.54 | 16.64 | >50.00 | 41.54 | >50.00 | 3.77 |
| SF-295 | 26.98 | >50.00 | 39.60 | >50.00 | 38.13 | 40.01 | 28.21 | >50.00 | >50.00 | >50.00 | 1.70 |
| SF-268 | 38.62 | >50.00 | 40.98 | 42.51 | 38.19 | 38.29 | 49.29 | >50.00 | >50.00 | >50.00 | 2.47 |

Lung

| | | | | | | | | | | | |
|---|---|---|---|---|---|---|---|---|---|---|---|
| H23 | 23.39 | >50.00 | 43.06 | 44.56 | 17.11 | 19.54 | 23.48 | >50.00 | >50.00 | >50.00 | 2.89 |

Kidney

| | | | | | | | | | | | |
|---|---|---|---|---|---|---|---|---|---|---|---|
| UO-31 | 20.56 | >50.00 | 9.20 | 9.96 | 13.93 | 16.82 | 8.27 | 34.57 | 15.70 | >50.00 | 0.81 |
| ACHN | 28.91 | >50.00 | 19.19 | 19.31 | 16.24 | 21.28 | 19.87 | >50.00 | 42.01 | >50.00 | 1.26 |

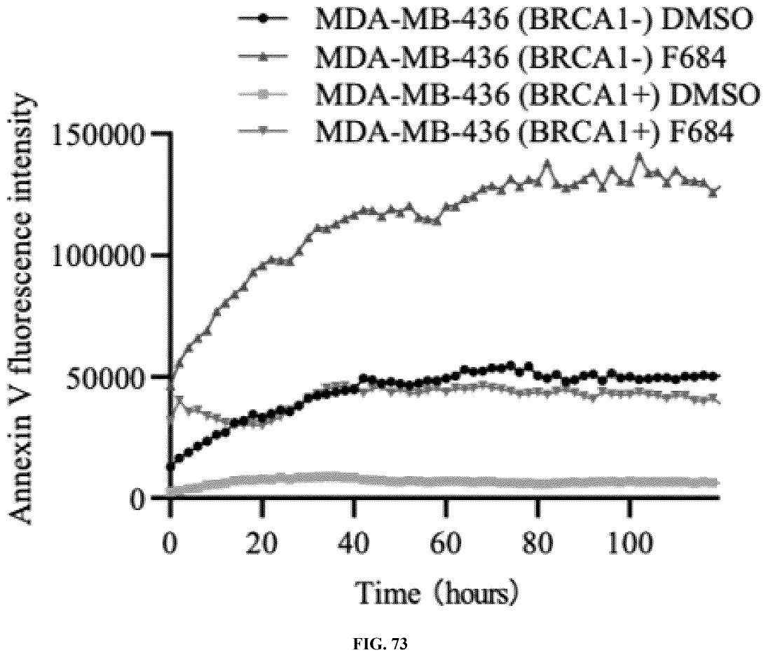

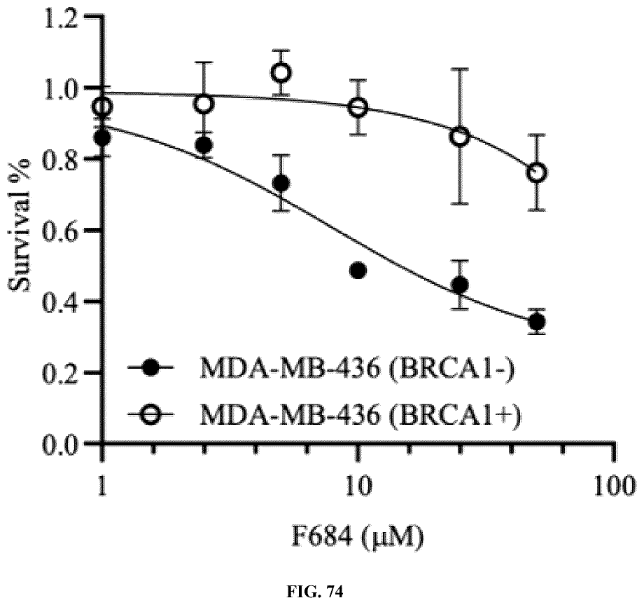

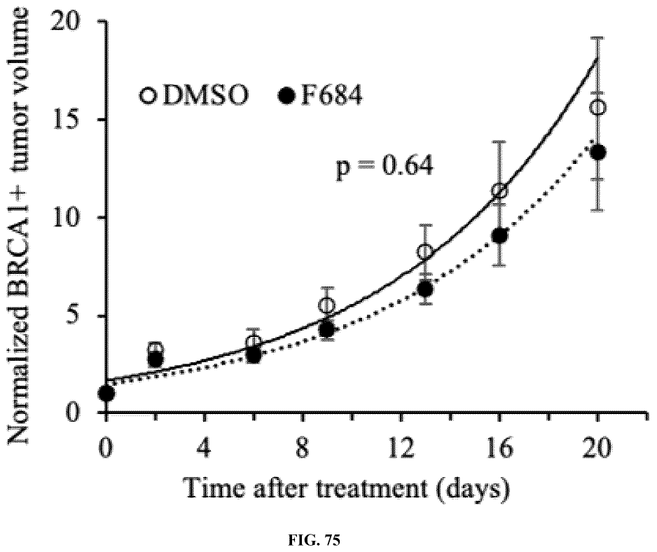

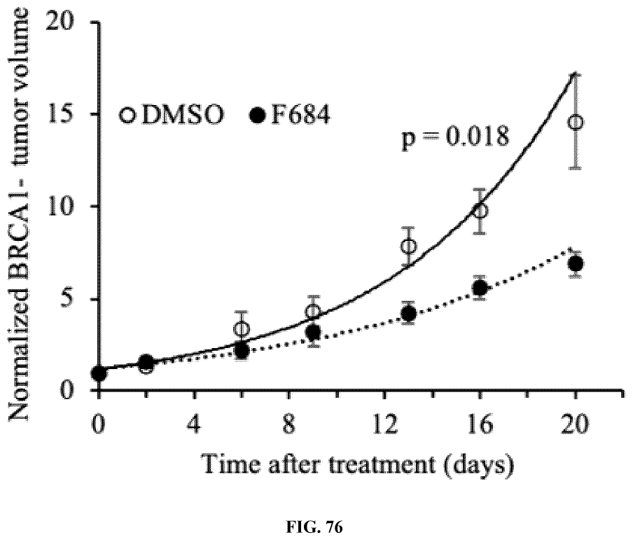

To further confirm that the cytotoxicity of compound F684 was selective for BRCA1-cells, Annexin V fluorescence intensity was measured over five days of treatment in WT BRCA1-expressing MDA-MB-436 (BRCA1+) and MDA-MB-436 parental (BRCA1−) cell lines. Compound F684 was substantially more effective at inducing apoptosis in parental MDA-MB-436 (BRCA1−) cells (red line) than WT BRCA1-expressing MDA-MB-436 (BRCA1+) cells (blue line). DMSO controls are shown in black and red lines, respectively (FIG. 73). The conclusions of the Annexin V assay were two-fold, 1) F684 treatment in MDA-MB-436 (BRCA1+) cells has a similar effect on cell survival as BRCA1−, and 2) F684 displays a substantially stronger killing effect in a BRCA1− context. Simultaneously, the killing effect of EXO1 inhibitor compound F684 was tested in vitro and mouse xenograft models using WT BRCA1-expressing MDA-MB-436 (BRCA1+) and MDA-MB-436 parental (BRCA1−) cell lines. In vitro, it was discovered that F684 treatment was more effective in killing BRCA1− cells compared to BRCA1+ cells. The cumulative survival percentage of MDA-MB-436 (BRCA1−) and MDA-MB-436 (BRCA1+) cells after treatment with 1, 2.5, 5, 10, 25, and 50 M concentrations of compound F684 is shown in FIG. 74. Error bars represent the standard deviation for each treatment. Compound F684 did not reduce the growth rate of BRCA1+ cancer cells in mouse tumor models. Tumor volumes in mouse xenograft models were established using WT BRCA1-expressing MDA-MB-436 (BRCA1+) and parental MDA-MB-436 (BRCA1−) cell lines (FIG. 74). Additionally, compound F684 significantly suppressed growth of BRCA1− tumors (FIG. 76), but had little effect in suppressing BRCA1+ tumors in vivo (FIG. 75). At 8.5 mg/kg body weight/day, compound F684 reduced the average growth rate of BRCA1− tumors from 15.7%/day to 11.3%/day (p=0.018), while it only reduced the average growth rate of BRCA1+ tumors from 16.6%/day to 15.1%/day (p=0.64). Together, these findings demonstrate the surprising and unexpected results the F684 scaffold provides as and EXO1 inhibitor.

Informal Sequence Listing

| SEQ | | |
|---|---|---|
| ID | | |
| NO. | Description | Sequence |
| 1 | Human EXO1 Wild- | MGIQGLLQFIKEASEPIHVRKYKGQVVAVDTYCWLH |
| | Type (Amino Acid) | KGAIACAEKLAKGEPTDRYVGFCMKFVNMLLSHGIK |
| | | PILVFDGCTLPSKKEVERSRRERRQANLLKGKQLLRE |
| | | GKVSEARECFTRSINITHAMAHKVIKAARSQGVDCLV |
| | | APYEADAQLAYLNKAGIVQAIITEDSDLLAFGCKKVI |
| | | LKMDQFGNGLEIDQARLGMCRQLGDVFTEEKFRYM |
| | | CILSGCDYLSSLRGIGLAKACKVLRLANNPDIVKVIK |
| | | KIGHYLKMNITVPEDYINGFIRANNTFLYQLVFDPIKR |
| | | KLIPLNAYEDDVDPETLSYAGQYVDDSIALQIALGNK |
| | | DINTFEQIDDYNPDTAMPAHSRSHSWDDKTCQKSAN |
| | | VSSIWHRNYSPRPESGTVSDAPQLKENPSTVGVERVIS |
| | | TKGLNLPRKSSIVKRPRSAELSEDDLLSQYSLSFTKKT |
| | | KKNSSEGNKSLSFSEVFVPDLVNGPTNKKSVSTPPRT |
| | | RNKFATFLQRKNEESGAVVVPGTRSRFFCSSDSTDCV |
| | | SNKVSIQPLDETAVTDKENNLHESEYGDQEGKRLVDT |
| | | DVARNSSDDIPNNHIPGDHIPDKATVFTDEESYSFESS |
| | | KFTRTISPPTLGTLRSCFSWSGGLGDFSRTPSPSPSTAL |
| | | QQFRRKSDSPTSLPENNMSDVSQLKSEESSDDESHPL |
| | | REEACSSQSQESGEFSLQSSNASKLSQCSSKDSDSEES |
| | | DCNIKLLDSQSDQTSKLRLSHFSKKDTPLRNKVPGLY |
| | | KSSSADSLSTTKIKPLGPARASGLSKKPASIQKRKHHN |
| | | AENKPGLQIKLNELWKNFGFKKDSEKLPPCKKPLSPV |
| | | RDNIQLTPEAEEDIFNKPECGRVQRAIFQ |
| 2 | FRET Assay Up-Primer | GCATCCTAAGCCATGCGTGGC |
| 3 | FRET Assay Template | CCTAAAAGTTTCACTCAGGACCACGCATGGCTTAG |
| | | GATGC |
| 4 | FRET Assay Probe | TCCTGAGTGAAACTTTTAGG |
| | (Raw Sequence) | |
| 5 | Lys85Ala Forward | TCTTCTAGATCTCTCTACTTCCTTTGCAGAAGGTAA |
| | Primer | AGTACATCCATCAAAT |
| 6 | Lys85Ala Reverse | ATTTGATGGATGTACTTTACCTTCTGCAAAGGAAGT |
| | Primer | AGAGAGATCTAGAAGA |
| 7 | Glu89Ala Forward | CGTCTTTCTCTTCTAGATCTCGCTACTTCCTTTTTAG |
| | Primer | AAGGTA |
| 8 | Glu89Ala Reverse | TACCTTCTAAAAAGGAAGTAGCGAGATCTAGAAGA |
| | Primer | GAAAGACG |
| 9 | Arg92Ala Forward | GATTGGCTTGTCGTCTTTCTCTTGCAGATCTCTCTAC |
| | Primer | TTCCTTTTTAG |
| 10 | Arg92Ala Reverse | CTAAAAAGGAAGTAGAGAGATCTGCAAGAGAAAG |
| | Primer | ACGACAAGCCAATC |
| 11 | Ile125Ala Forward | GGGCCATGGCATGTGTGGCATTGATAGACCGGGTG |
| | Primer | AA |
| 12 | Ile125Ala Reverse | TTCACCCGGTCTATCAATGCCACACATGCCATGGCCC |
| | Primer | |
| 13 | Asp225Ala Forward | CCCGGTCTATCAATGCCACACATGCCATGG |
| | Primer | |
| 14 | Asp225Ala Reverse | CCATGGCATGTGTGGCATTGATAGACCGGG |
| | Primer | |
| 15 | Lys292Ala/Lys294Ala | CTTCATAGGCGTTCAGAGGAATAAGTGCCCTTGCGA |
| | Forward Primer | TGGGATCAAAAACTAGCTGATAGA |
| 16 | Lys292Ala/Lys294Ala | TCTATCAGCTAGTTTTTGATCCCATCGCAAGGGCAC |
| | Reverse Primer | TTATTCCTCTGAACGCCTATGAAG |

Annotations for SEQ ID NO: 1 (EXO1 WT)

| Mutant | Feature Key | Location | Qualifier Note |
|---|---|---|---|
| EXO1 K54A | variant | 85 | K85A: Lysine (K) replaced by Alanine (A) |
| EXO1 E89A | variant | 89 | E89A: Glutamic Acid (E) replaced by Alanine (A) |
| EXO1 R92A | variant | 92 | R92A: Arginine (R) replaced by Alanine (A) |
| EXO1 I125A | variant | 125 | I125A: Isoleucine (I) replaced by Alanine (A) |
| EXO1 D225A | variant | 225 | D225A: Aspartic Acid (D) replaced by Alanine (A) |
| EXO1 K292A | variant | 292 | K292A: Lysine (K) replaced by Alanine (A) |
| EXO1 K294A | variant | 294 | K294A: Lysine (K) replaced by Alanine (A) |

Annotations for SEQ ID NO: 4 (Probe)

| Feature | Location | Qualifier (/note) |
|---|---|---|
| 5′ 6-FAM | 1 | 5′ 6-carboxyfluorescein |
| | | (6-FAM) fluorophore |
| ZEN quencher | 9 . . . 10 | Internal ZEN ® quencher attached |
| | | between nucleotides 9 and 10 |
| 3′ Iowa | 20 | 3′ Iowa Black ® FQ |
| Black ® FQ | | (3IABkFQ) quencher |

What is claimed is:

1. A human exonuclease 1 inhibitor compound of Formula (I): 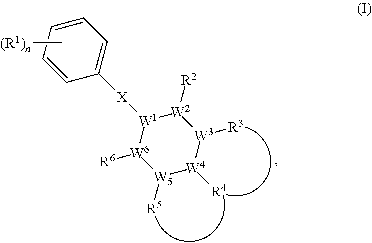
or a pharmaceutically acceptable salt thereof;
wherein X is a bond, —NH—, or —NH— alkyl-;
R¹ is absent, H, halo, or L;
R² absent, H, halo, or L;
R³ is H, halo, —OH, alkyl, bound to R⁴, or L;
R⁴ is H, halo, —OH, alkyl-L, bound to R³, bound to R⁵, or L;
R⁵ is absent, H, ═O, —C(O)O-alkyl, bound to R⁴, or L;
R⁶ is absent, H, or L;
W² is —CH—, —N═, —NH—;
W³ is —CH—, —C═;
W⁴ is —N—, —CH—, —C═;
W⁵ is —CH—, —C—, —C═;
W⁶ is —CH—, —C═, —N═;
L is ═O, —C(O)OH, alkyl, aryl, benzyl, —O-alkyl, —NHC(O)Y, —NHS(O)₂Y, —SCH₂C(O)O—Y;
Y is substituted or unsubstituted aryl, substituted or unsubstituted heteroaryl, substituted or unsubstituted heterocycle,
when R³ is bound to R⁴ or R⁴ is bound to R⁵ a five membered unsubstituted or L-substituted heterocycle is formed; and
n is 0-5.

2. The human exonuclease 1 inhibitor of claim 1, wherein the compound has the structure of Formula (II):

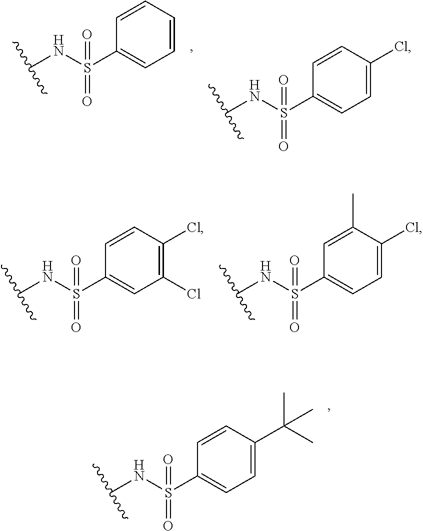

3. The human exonuclease 1 inhibitor of claim 1, wherein L is:

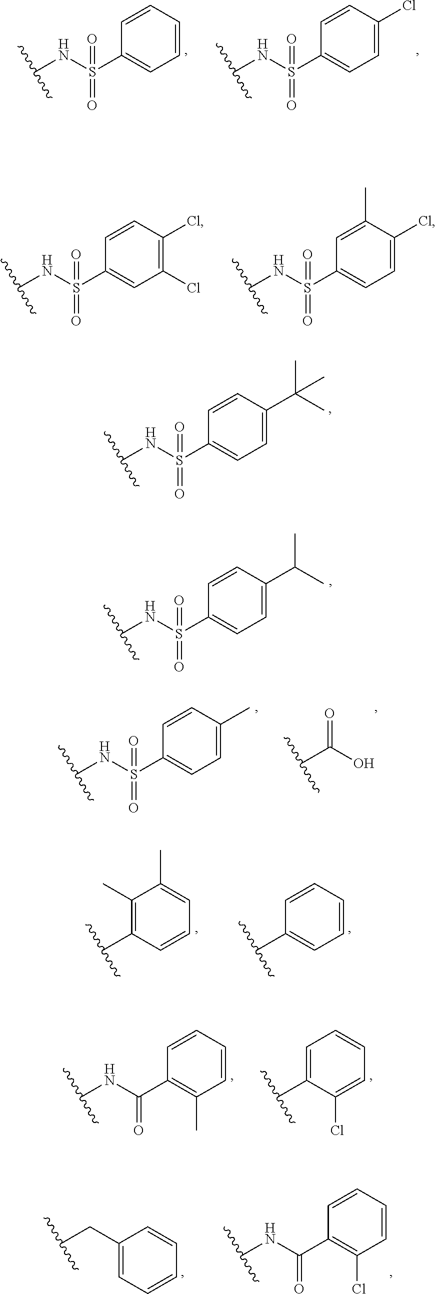

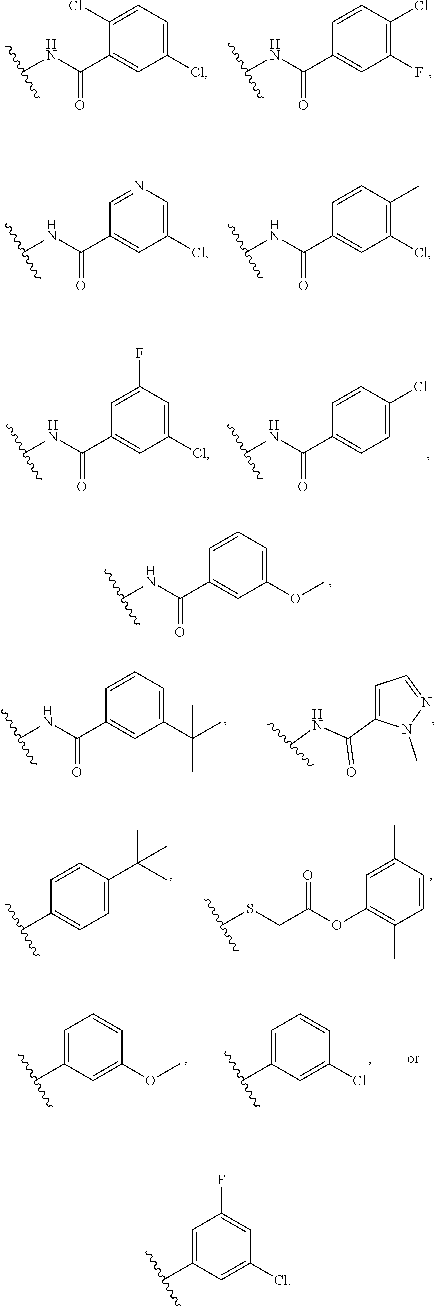

4. The human exonuclease 1 inhibitor of claim 1, wherein Y is:

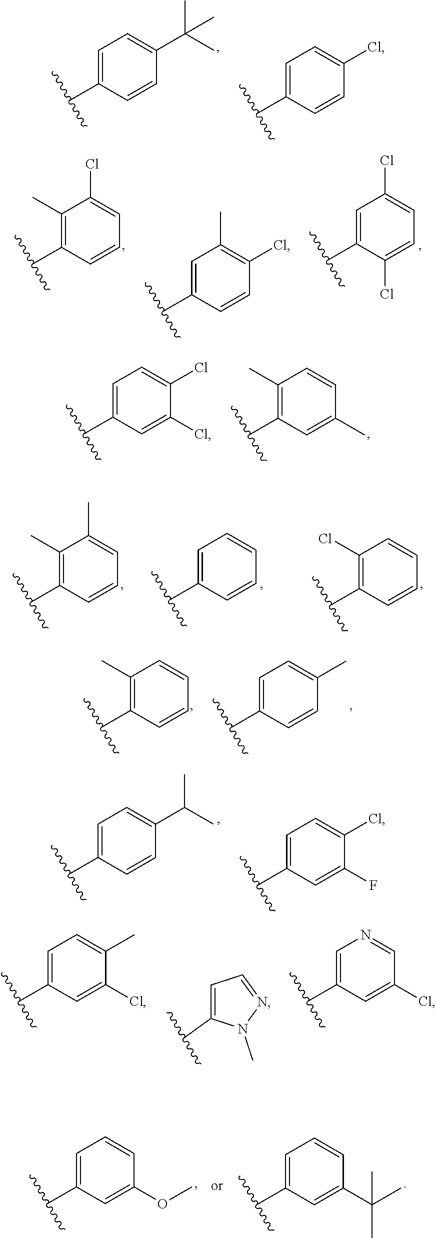

5. The human exonuclease 1 inhibitor of claim 1, wherein the compound is: 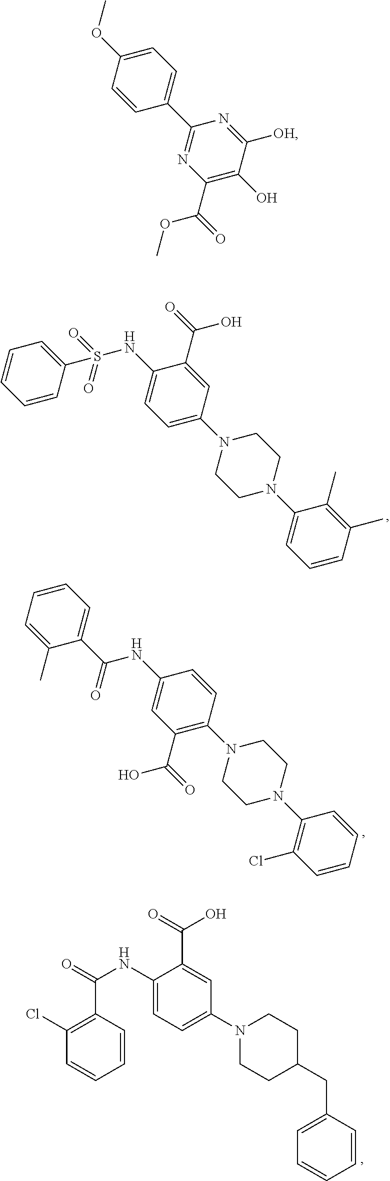 [SMILES: COC(=O)c1nc(-c2ccc(OC)cc2)nc(O)c1O ; Cc1cccc(N2CCN(c3ccc(NS(=O)(=O)c4ccccc4)c(C(=O)O)c3)CC2)c1C ; Cc1ccccc1C(=O)Nc1ccc(N2CCN(c3ccccc3Cl)CC2)c(C(=O)O)c1 ; O=C(Nc1ccc(N2CCC(Cc3ccccc3)CC2)cc1C(=O)O)c1ccccc1Cl] 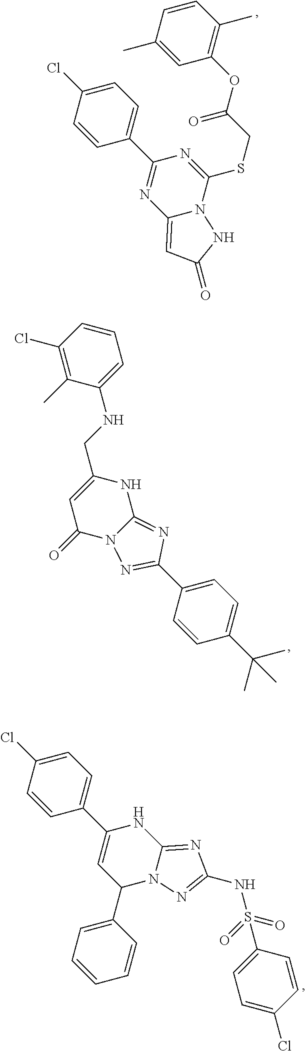
or a pharmaceutically acceptable salt thereof.

6. The human exonuclease 1 inhibitor of claim 1, wherein the compound is: 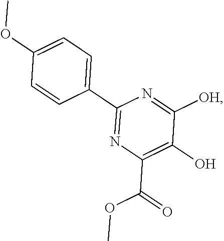 [SMILES: COC(=O)c1nc(-c2ccc(OC)cc2)nc(O)c1O]
or a pharmaceutically acceptable salt thereof.

7. The human exonuclease 1 inhibitor of claim 1, wherein the compound is: 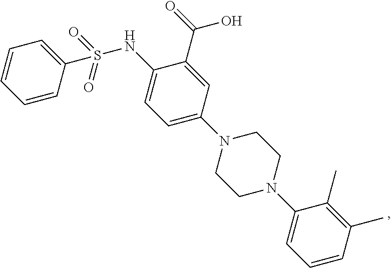 [SMILES: Cc1cccc(N2CCN(c3ccc(NS(=O)(=O)c4ccccc4)c(C(=O)O)c3)CC2)c1C]
or a pharmaceutically acceptable salt thereof.

8. A pharmaceutical composition comprising the human exonuclease 1 inhibitor compound of claim 1 and a pharmaceutically acceptable excipient.

9. A method of treating cancer in a subject, comprising administering to the subject the human exonuclease 1 inhibitor compound of claim 1.

10. The method of claim 9, wherein the cancer is breast, ovarian, prostate, leukemia, melanoma, pancreatic, central nervous system, lung, or kidney cancer.

11. The method of claim 9, wherein the compound has a IC₅₀ of about 1.0 μM to about 65.0 μM against human exonuclease 1 protein.

12. The method of claim 9, wherein administration of the compound is intracutaneous, subcutaneous, intravenous, intraperitoneal, intraarterial, intrathecal, intracapsular, intraorbital, intracardiac, intradermal, transdermal, transtracheal, subcuticular, intraarticular, subcapsular, subarachnoidal, intraspinal, intrasternal, oral, sublingual, buccal, rectal, vaginal, nasal or ocular, or by infusion, inhalation, or nebulization.

13. The method of claim 9, wherein a therapeutically effective amount of the compound is administered to the subject.

14. The method of claim 9, wherein the compound inhibits cancer cell proliferation, cancer cell viability or a combination thereof.

15. A kit comprising the exonuclease 1 inhibitor compound of claim 1 and instructions for use.


SEQUENCE LISTING (16 sequences)

SEQ ID NO: 1 - protein, 846 aa, Homo sapiens
  VARIANT 85: note = K85A: Lysine (K) replaced by Alanine (A)
  VARIANT 89: note = E89A: Glutamic Acid (E) replaced by Alanine (A)
  VARIANT 92: note = R92A: Arginine (R) replaced by Alanine (A)
  VARIANT 125: note = I125A: Isoleucine (I) replaced by Alanine (A)
  VARIANT 225: note = D225A: Aspartic Acid (D) replaced by Alanine (A)
  VARIANT 292: note = K292A: Lysine (K) replaced by Alanine (A)
  VARIANT 294: note = K294A: Lysine (K) replaced by Alanine (A)
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 2 - DNA, 21 bp, Homo sapiens
  source 1..21: mol_type = other DNA
gcatcctaagccatgcgtggc

SEQ ID NO: 3 - DNA, 40 bp, Homo sapiens
  source 1..40: mol_type = other DNA
cctaaaagtttcactcaggaccacgcatggcttaggatgc

SEQ ID NO: 4 - DNA, 20 bp, Homo sapiens
  source 1..20: mol_type = other DNA
  modified_base 1: mod_base = OTHER; note = 5' 6-FAM: 6-carboxyfluorescein fluorophore attached to the 5' phosphate or the first nucleotide
  modified_base 9^10: mod_base = OTHER; note = Internal ZEN: Proprietary ZEN quencher inserted internally between the 9th and 10th nucleotides (G and A)
  modified_base 20: mod_base = OTHER; note = 3' IABkFQ: Iowa Black FQ quencher attached to the 3' end of the last nucleotide (G)
tcctgagtgaaacttttagg

SEQ ID NO: 5 - DNA, 52 bp, Homo sapiens
  source 1..52: mol_type = other DNA
tcttctagatctctctacttcctttgcagaaggtaaagtacatccatcaaat

SEQ ID NO: 6 - DNA, 52 bp, Homo sapiens
  source 1..52: mol_type = other DNA
atttgatggatgtactttaccttctgcaaaggaagtagagagatctagaaga

SEQ ID NO: 7 - DNA, 43 bp, Homo sapiens
  source 1..43: mol_type = other DNA
cgtctttctcttctagatctcgctacttcctttttagaaggta

SEQ ID NO: 8 - DNA, 43 bp, Homo sapiens
  source 1..43: mol_type = other DNA
taccttctaaaaaggaagtagcgagatctagaagagaaagacg

SEQ ID NO: 9 - DNA, 48 bp, Homo sapiens
  source 1..48: mol_type = other DNA
gattggcttgtcgtctttctcttgcagatctctctacttcctttttag

SEQ ID NO: 10 - DNA, 48 bp, Homo sapiens
  source 1..48: mol_type = other DNA
ctaaaaaggaagtagagagatctgcaagagaaagacgacaagccaatc

SEQ ID NO: 11 - DNA, 37 bp, Homo sapiens
  source 1..37: mol_type = other DNA
gggccatggcatgtgtggcattgatagaccgggtgaa

SEQ ID NO: 12 - DNA, 37 bp, Homo sapiens
  source 1..37: mol_type = other DNA
ttcacccggtctatcaatgccacacatgccatggccc

SEQ ID NO: 13 - DNA, 30 bp, Homo sapiens
  source 1..30: mol_type = other DNA
cccggtctatcaatgccacacatgccatgg

SEQ ID NO: 14 - DNA, 30 bp, Homo sapiens
  source 1..30: mol_type = other DNA
ccatggcatgtgtggcattgatagaccggg

SEQ ID NO: 15 - DNA, 60 bp, Homo sapiens
  source 1..60: mol_type = other DNA
cttcataggcgttcagaggaataagtgcccttgcgatgggatcaaaaactagctgataga

SEQ ID NO: 16 - DNA, 60 bp, Homo sapiens
  source 1..60: mol_type = other DNA
tctatcagctagtttttgatcccatcgcaagggcacttattcctctgaacgcctatgaag